(19) United States
(12) Patent Application Publication
(10) Pub. No.: US 2016/0120959 A1
(43) Pub. Date: May 5, 2016

(54) Use of Klotho Nucleic Acids or Proteins for Treatment of Diabetes and Diabetes-Related Conditions

(71) Applicant: THE BOARD OF REGENTS OF THE UNIVERSITY OF OKLAHOMA, Norman, OK (US)

(72) Inventor: Zhongjie SUN, Edmond, OK (US)

(21) Appl. No.: 14/776,452

(22) PCT Filed: Mar. 14, 2014
(86) PCT No.: PCT/US14/28563

§ 371 Date: Sep. 14, 2015

Related U.S. Application Data
(60) Provisional application No. 61/784,640, filed on Mar. 14, 2013.

(51) Int. Cl. A61K 38/47; C12N 7/00

(52) U.S. Cl. CPC A61K 38/47; C12N 7/00; A61K 48/00

(57) ABSTRACT

In one embodiment, the inventive concepts include a method of treating a diabetic condition or a diabetes-related condition in a subject in need of such treatment by administering to the subject a therapeutically-effective amount of a vector comprising a nucleic acid which encodes a klotho protein or a therapeutically-effective portion of a klotho protein, wherein the klotho protein or therapeutically-effective portion of the klotho protein is expressed in vivo in pancreatic beta cells of the subject. In another embodiment, the inventive concepts include a method of treating a diabetic condition or a diabetes-related condition in a subject in need of such treatment by administering to the subject a therapeutically-effective amount of at least one of a klotho protein and a therapeutically-effective portion of the klotho protein.

### CROSS REFERENCE TO RELATED APPLICATIONS/INCORPORATION BY REFERENCE STATEMENT

This application claims benefit under 35 USC §119(e) of provisional application U.S. Ser. No. 61/784,640, filed Mar. 14, 2013, the entirety of which is hereby expressly incorporated herein by reference.

### STATEMENT REGARDING FEDERALLY SPONSORED RESEARCH OR DEVELOPMENT

This invention was made with government support under Contract Numbers HL105302, HL102074, and DK 093403 awarded by the National Institutes of Health (NIH). The government has certain rights in the invention.

### BACKGROUND

Diabetes affects about 150 million people worldwide, and this figure is expected to double in the next 20 years. About 8% of the US population has diabetes. Of these, about 90-95% of all diabetes cases are type 2 diabetes mellitus (T2DM), and about 5-10% are type 1 diabetes mellitus (T1DM). Physiologically, the pancreatic β cells constantly synthesize insulin. Insulin is stored within vacuoles and released when triggered by an elevation of the blood glucose level. Insulin is the principal hormone that regulates uptake of glucose from the blood into most cells, including skeletal muscle cells and adipocytes. Insulin is also the major signal for the conversion of glucose to glycogen for internal storage in liver and skeletal muscle cells. T2DM was for many years recognized only because of insulin resistance. There is now a common agreement that T2DM is a complex pathophysiologic spectrum, including insulin resistance and β cell failure. Significant β cell failure is now believed to take place at an early stage in disease progression, i.e., β cell function declines sharply before and after diagnosis of T2DM. In the United Kingdom Prospective Diabetes Study, for example, the secretory capacity of β cells was reduced by 50% at the time fasting hyperglycemia was diagnosed.

Generally, the β cell compensatory ability with respect to an increase in insulin resistance keeps blood glucose at the near-normal level through proportionate enhancements of β cell function. It has been shown that there is no hyperglycemia without β cell dysfunction. Maintaining recommended targets of blood glucose control is difficult for many patients with T2DM due to the progressive loss of β cell function. T1DM results from immune-mediated destruction of insulin-producing pancreatic β cells. It has been estimated that at the time of diagnosis, patients with T1DM suffer from about 60-80% reduction in 0 cell mass. It has been shown that β cell apoptosis causes a gradual β cell depletion in rodent models of T1DM. Both direct cytotoxic (T-cell mediated) and indirect cytokine-dependent (e.g., tumor necrosis factor-α) mechanisms are considered to be responsible for β cell apoptosis. Thus, one of the goals in the treatment of T2DM and T1DM is the preservation of functional β cells in pancreatic islets.

Klotho is a recently identified anti-aging gene. The mouse Klotho (also called α Klotho) gene contains 5 exons and encodes a single-pass transmembrane protein with 1014 amino acids, predominantly expressed in the kidney and the brain choroid plexus. The majority of amino acids in the Klotho protein reside in the amino-terminal extracellular domain, which is followed by a 21 amino acid trans-membrane domain, and an 11 amino acid short intracellular carboxyl terminus. There are two forms of Klotho protein, the full-length Klotho (130 kDa) and the short-form Klotho (65 kDa), which can be generated by alternative RNA splicing or proteolytic cleavage. Overexpression of Klotho extended life span in mice, whereas mutation of Klotho gene caused multiple premature-aging phenotypes and shortened lifespan. Klotho has been reported to function as a cofactor for activation of FGFR1c by FGF23 in the regulation of calcium, phosphate, and vitamin D metabolism in kidneys. It is noted that Klotho^{-/-} mice display pancreatic islet atrophy, decreases in insulin content, decreases in mRNA levels in pancreatic islets, and decreases in serum insulin levels.

### BRIEF DESCRIPTION OF THE DRAWINGS

The following drawings depict certain embodiments of the presently disclosed inventive concepts. They are illustrative only and do not limit the presently disclosed inventive concepts otherwise disclosed herein.

FIG. 1 illustrates expressions of Klotho in pancreatic islets of patients with T2DM and db/db mice (20 weeks-old). A. Representative images of Klotho and insulin staining (brown color) in cross-sections of human pancreatic islets. B. Semi-quantification of Klotho and insulin staining in human pancreatic islets. n=4 to 6; *p<0.05 vs the normal samples. C. Representative images of Klotho staining (brown color) in cross-sections of mouse pancreatic islets. D. Representative images of insulin staining (brown color) in cross-sections of mouse pancreatic islets. E. Western blot analysis of Klotho protein expression in human pancreas. n=4 to 5. **p<0.01, ***p<0.001 vs the lean mice. Results were standardized to β-actin.

FIG. 2 shows expressions of Klotho in pancreatic islets of patients with T2DM and db/db mice (20 weeks-old). A. Semi-quantification of Klotho staining in mouse pancreatic islets. B. Semi-quantification of insulin staining in mouse pancreatic islets. C,D. Western blot analysis of Klotho protein expression in mouse pancreas. Results were standardized to β-actin vs the lean mice. Data=means±SEM. n=4 to 5. **p<0.01 vs the lean mice.

FIG. 3 illustrates expression of GFP or Klotho in MIN6 β cells. MIN6 cells, 3T3-L1 preadipocytes, and mlMCDs cells were transfected with pAAV-GFP for 48 hours. Images were collected with a 10× objective lens. A. Phase contrast image of 3T3-L1 preadipocytes (upper photo); fluorescent image of 3T3-L1 cells (lower photo). B. Phase contrast image of mlMCD3 cells (upper photo); fluorescent image of mlMCDs cells (lower photo). C. Phase contrast image of MIN6 β cells (upper photo); fluorescent image of MIN6 cells (lower photo). D. Western blot analysis of Klotho protein expression (full length and short form) in MIN6 β cells. MIN6 cells were incubated with transfection reagent alone, pAAV-mKL, or pAAV-CMV-mKL for 48 hours, respectively. The cell lysates were directly subjected to SDS-PAGE followed by western blotting with antibody against Klotho. The blot was rinsed and reprobed with antibody against β-actin.

FIG. 4 illustrates the effects of the β cell-specific expression of mKL on blood glucose levels and glucose tolerance in diabetic mice. The time course of fasting blood glucose levels (4a). Glucose tolerance test (GTT) results at week 2 (4b) after gene delivery. Area under the curve for GTT results at week 2 (4c) after gene delivery.

FIG. 5 illustrates the effects of the β cell-specific expression of mKL on blood glucose levels and glucose tolerance in diabetic mice. Glucose tolerance test (GTT) results at week 4 (5a) and week 6 (5c) after gene delivery. Area under the curve for GTT results at week 4 (5b) and week 6 (5d) after gene delivery.

FIG. 6 illustrates the effects of the β cell-specific expression of mKL on blood glucose levels and insulin sensitivity in diabetic mice. Original readings of insulin sensitivity test (IST) results at week 3 (6a) and week 5 (6c) after the treatments. Normalized blood glucose levels of IST results at week 3 (6b) and week 5 (6d) after gene delivery.

FIG. 7 illustrates the effects of the β cell-specific expression of mKL on urine glucose levels and plasma insulin levels in diabetic mice. Plasma insulin levels at week 3 after gene delivery (7a). Plasma insulin levels in mice at week 6 after gene delivery (7b). Urine glucose levels at week 2 and 4 after gene delivery (7c). Data=mean±SEM. n=6-8 animals/group (except for plasma samples n=3 to 5). *p<005, **p<0.01, ***p<0.001 vs the lean-PBS group; ⁺⁺p<0.01, ⁺⁺⁺p<0.001 vs the db/db-PBS group.

FIG. 8 shows expressions of Klotho and insulin in pancreatic islets and analysis of the islet function in diabetic mice. Animals were sacrificed 6 weeks after gene delivery. Representative images of Klotho staining (brown color) in cross-sections of mouse pancreatic islets. Data=mean±SEM, n=3 animals/group. *** p<0.001 vs the lean-PBS group treated with 2.8 mM glucose; ̂̂̂<0.001 vs the db/db-PBS group treated with 2.8 mM glucose; ###p<0.001 vs the db/db-PBS group treated with 16.7 mM glucose; ++p<0.01 vs the lean-PBS group treated with 16.7 mM glucose.

FIG. 9 shows expressions of Klotho and insulin in pancreatic islets and analysis of the islet function in diabetic mice of FIG. 8. Representative images of insulin staining (brown color) in cross-sections of islets.

FIG. 10 shows expressions of Klotho and insulin in pancreatic islets and analysis of the islet function in diabetic mice of FIG. 8. A: Semi-quantification of Klotho staining in pancreatic islets (n=4 to 5). B: Semi-quantification of insulin staining in pancreatic islets (n-=4 to 5). C: The percentage of insulin-positive cells in pancreatic islets (n=4 to 5). ***p<0.001 vs the lean-PBS group; ⁺⁺⁺p<0.001 vs the db/db-PBS group. D: Glucose-stimulated insulin secretion from pancreatic islets.

FIG. 11 illustrates oxidative stress, superoxide, and Pdx-1 levels in pancreatic islets of diabetic mice. Representative images of 4-HNE staining (indicated by arrows, brown color) in cross-sections of mouse pancreatic islets. Data=mean±SEM, n=4-5 animals/group. ***p<0.001 vs the lean-PBS group; ⁺p<0.05, ⁺⁺⁺p<0.001 vs the db/db-PBS group.

FIG. 12 illustrates superoxide levels in pancreatic islets of diabetic mice. Representative images of DHE staining (red color) in pancreatic islets. Data=mean±SEM, n=4-5 animals/group. ***p<0.001 vs the lean-PBS group; ⁺p<0.05, **p<0.001 vs the db/db-PBS group.

FIG. 13 illustrates Pdx-1 levels in pancreatic islets of diabetic mice. Representative images of Pdx-1 staining in pancreatic islets. Data=mean±SEM, n=4-5 animals/group. ***p<0.001 vs the lean-PBS group; ⁺p<0.05, ⁺⁺⁺p<0.001 vs the db/db-PBS group.

FIG. 14 illustrates oxidative stress, superoxide, and Pdx-1 levels in pancreatic islets of diabetic mice. a: The percentage of 4-HNE-positive cells in pancreatic islets. b: Quantification of superoxide levels (DHE staining) in pancreatic islets. c: Semi-quantification of Pdx-1 staining in pancreatic islets. Data=mean±SEM, n=4-5 animals/group. ***p<0.001 vs the lean-PBS group; ⁺p<0.05, ⁺⁺⁺p<0.001 vs the db/db-PBS group.

FIG. 15 shows the effects of β cell-specific expression of mKL on ER stress and autophagy in pancreatic islets. Representative images of DNAJC3 staining (indicated by arrows, brown color) in mouse pancreatic islets. Data=mean±SEM, n=4-5 animals/group. **p<0.01, ***p<0.001 vs the lean-PBS group; ⁺⁺p<0.01, ⁺⁺⁺p<0.001 vs the db/db-PBS group.

FIG. 16: shows the effects of R cell-specific expression of mKL on ER stress and autophagy in pancreatic islets. Representative images of LC3 staining (brown color) in cross-sections of mouse pancreatic islets. Data=mean±SEM, n=4-5 animals/group. **p<0.01, ***p<0.001 vs the lean-PBS group; ⁺⁺p<0.01, ⁺⁺⁺p<0.001 vs the db/db-PBS group.

FIG. 17 shows the effects of β cell-specific expression of mKL on ER stress and autophagy in pancreatic islets. 17a: The percentage of DNAJC3 positive-staining cells in pancreatic islets. 17b: Semi-quantification of LC3 staining in pancreatic islets. Data=mean±SEM, n-4-5 animals/group. **p<0.01, ***p<0.001 vs the lean-PBS group; ⁺⁺p<0.01, ⁺⁺⁺p<0.001 vs the db/db-PBS group.

FIG. 18 shows the effects of β cell-specific expression of mKL on cell proliferation and apoptosis in pancreatic islets. Representative images of PCNA staining (indicated by arrows, brown color) in mouse pancreatic islets. Data=mean±SEM, n=4-5 animals/group. ***p<0.001 vs the lean-PBS group; ⁺⁺p<0.01, ⁺⁺⁺p<0.001 vs the db/db-PBS group.

FIG. 19 shows the effects of β cell-specific expression of mKL on cell proliferation and apoptosis in pancreatic islets. Representative images of TUNEL staining (indicated by arrows, blue color) in pancreatic islets. Data=mean±SEM, n=4-5 animals/group. ***p<0.001 vs the lean-PBS group; ⁺⁺p<0.01, ⁺⁺⁺p<0.001 vs the db/db-PBS group.

FIG. 20 shows the effects of β cell-specific expression of mKL on cell proliferation and apoptosis in pancreatic islets. 20a: The percentage of PCNA-positive cells in pancreatic islets. 20b: The percentage of TUNEL-positive apoptotic cells in pancreatic islets. Data=mean±SEM, n=4-5 animals/group. ***p<0.001 vs the lean-PBS group; ⁺⁺p<0.01, ⁺⁺⁺p<0.001 vs the db/db-PBS group.

FIG. 21 illustrates gene expression in islets isolated from diabetic mice. Real time RT-PCR analysis of mRNA expression of insulin I (A), insulin II (B), and Pdx-1(C). Results were standardized to β-actin mRNA levels and then expressed as fold changes vs the lean-PBS mice. Data=mean±SEM, n=3 animals/group. *p<0.05, **p<0.01, ***p<0.001 vs the lean-PBS group; ⁺p<0.05, ⁺⁺p<0.01, ⁺⁺⁺p<0.001 vs the db/db-PBS group.

FIG. 22 illustrates gene expression in islets isolated from diabetic mice. Real time RT-PCR analysis of mRNA expression of PCNA (A), DNAJC3 (B), and LC3 (C) are shown. Results were standardized to β-actin mRNA levels and then expressed as fold changes vs the lean-PBS mice. Data=mean±SEM, n=3 animals/group. *p<0.05, **p<0.01, ***p<0.001 vs the lean-PBS group; ⁺p<0.05, ⁺⁺p<0.01, ⁺⁺⁺p<0.001 vs the db/db-PBS group.

FIG. 23 illustrates the effects of the β cell-specific expression of mKL on metabolic parameters in diabetic mice. Food intake, water intake, and urine output were measured using metabolic cages during weeks 1, 3 and 5 following gene delivery. 23: Weekly body weights (A). Food intake at week 1 (B), week 3 (C), and week 5 (D) after gene delivery. FIG. 23 illustrates the effects of the β cell-specific expression of mKL on metabolic parameters in diabetic mice. Food intake, water intake, and urine output were measured using metabolic cages during weeks 1, 3 and 5 following gene delivery.

FIG. 24 illustrates the effects of the β cell-specific expression of mKL on metabolic parameters in diabetic mice. Food intake, water intake, and urine output were measured using metabolic cages during weeks 1, 3 and 5 following gene delivery. Water intake at week 1 (A), week 3 (B), and week 5 (C) after the injections.

FIG. 25 illustrates the effects of the β cell-specific expression of mKL on metabolic parameters in diabetic mice. Food intake, water intake, and urine output were measured using metabolic cages during weeks 1, 3 and 5 following gene delivery. Urine output at week 1 (A), week 3 (B), and week 5 (C) after gene delivery. Data=mean±SEM. n=6-8 animals/group. **p<0.01, ***p<0.001 vs the lean-PBS group; ⁺p<0.05, ⁺⁺p<0.01, ⁺⁺⁺p<0.001 vs the db/db-PBS group.

FIG. 26 illustrates the identification of GFP or FLAG-tag in mouse pancreatic islets of Langerhans and measurement of Klotho protein expression in mouse pancreas. At 6 weeks after the gene delivery, animals were sacrificed and mouse pancreases, livers, and kidneys were collected, followed by paraffin embedding. A part of pancreas was processed for western blot analysis of Klotho expression. A: Representative images of GFP staining (brown color) in cross-sections of pancreatic islets. GFP protein was detected using the antibody against GFP. B: Representative images of GFP staining (brown color) in cross-sections of the liver and Kidney of lean mice treated with rAAV-GFP.

FIG. 27 illustrates the identification of GFP or FLAG-tag in mouse pancreatic islets of Langerhans and measurement of Klotho protein expression in mouse pancreas. At 6 weeks after the gene delivery, animals were sacrificed and mouse pancreases, livers, and kidneys were collected, followed by paraffin embedding. A part of pancreas was processed for western blot analysis of Klotho expression. Representative images of FLAG-tag staining (brown color) in cross-sections of mouse pancreatic islets. The 3′ end of mouse KL gene was coupled with Flag-tag gene in the construct of rAAV-mKL FLAG-tag was detected using the antibody against FLAG-tag.

FIG. 28 illustrates the identification of GFP or FLAG-tag in mouse pancreatic islets of Langerhans and measurement of Klotho protein expression in mouse pancreas. At 6 weeks after the gene delivery, animals were sacrificed and mouse pancreases, livers, and kidneys were collected, followed by paraffin embedding. A part of pancreas was processed for western blot analysis of Klotho expression. A. Western blot analysis of Klotho protein expression in mouse pancreas. B: Quantification of short form Klotho in mouse pancreas. Results were standardized to β-actin and then expressed as fold changes vs the lean-PBS group. Data=means±SEM. n=3 to 5. **p<0.01, ***p<0.001 vs the lean-PBS group; ++p<0.01 vs db/db-PBS group.

FIG. 29 illustrates that the half deficiency of Klotho in KL^{+/-} mice exacerbated the development of T1DM induced by STZ. KL^{+/-} and wild type male mice were injected with STZ or citrate buffer. Blood glucose levels, glucose tolerance, insulin sensitivity, and plasma insulin levels were measured during the 5-week period. A. Fasting blood glucose levels. B and D. Glucose tolerance test (GTT) results at week 2 and week 4 after the initial STZ injections. C and E. Area under the curve for GTT results at week 2 and week 4 after the initial STZ injections.

FIG. 30 illustrates that the half deficiency of Klotho in KL^{+/-} mice exacerbated the development of T1DM induced by STZ. KL^{+/-} and wild type male mice were injected with STZ or citrate buffer. Blood glucose levels, glucose tolerance, insulin sensitivity, and plasma insulin levels were measured during the 5-week period. A. Original readings of insulin sensitivity test (IST) results at week 5 after the initial STZ injections. B. Normalized blood glucose levels of IST results at week 5 after the initial injections. C. Plasma insulin levels at week 5 after the injections. D. Urine glucose levels after the injections. Data=mean±SEM. n=4-8 animals/group, **p<0.01, ***p<0.001 vs the wild type group; ⁺p<0.05, ⁺⁺p<0.01 vs the wild type-STZ group.

FIG. 31 shows immunohistochemical analysis of Klotho and insulin expression and apoptosis in pancreatic islets of mice treated with STZ. KL^{+/-}, and wild type male mice were injected with STZ or citrate buffer. Animals were sacrificed 5 weeks after the initial injections. Representative images of Klotho staining (brown color) in cross-sections of mouse pancreatic islets.

FIG. 32 shows immunohistochemical analysis of Klotho and insulin expression and apoptosis in pancreatic islets of mice treated with STZ. KL^{+/-}, and wild type male mice were injected with STZ or citrate buffer. Animals were sacrificed 5 weeks after the initial injections. Representative images of insulin staining (brown color) of cross-sections of islets.

FIG. 33 shows immunohistochemical analysis of Klotho and insulin expression and apoptosis in pancreatic islets of mice treated with STZ. KL^{+/-}, and wild type male mice were injected with STZ or citrate buffer. Animals were sacrificed 5 weeks after the initial injections. Representative images of TUNEL staining (blue color) in pancreatic islets.

FIG. 34 shows immunohistochemical analysis of Klotho and insulin expression and apoptosis in pancreatic islets of mice treated with STZ. KL^{+/-}, and wild type male mice were injected with STZ or citrate buffer. Animals were sacrificed 5 weeks after the initial injections. a: Semi-quantification of Klotho staining in pancreatic islets. b: Semi-quantification of insulin staining in pancreatic islets. c: The percentage of cells with positive insulin-staining in pancreatic islets. d: The number of TUNEL-positive apoptotic cells in pancreatic islets. Data=mean±SEM. N=4-8 animals/group. **p<0.05, ***p<0.001 vs the wild type group; ⁺⁺p<0.01, ⁺⁺⁺p<0.001 vs the wild type-STZ group.

FIG. 35 illustrates the effects of the β cell-specific expression of mKL on blood glucose levels, glucose tolerance, insulin sensitivity, and plasma insulin levels in STZ-induced diabetic mice. 129S1/Svlm male mice were injected with PBS, rAAV-GFP, or rAAV-mKL respectively. One week after gene delivery, these mice were injected with STZ or citrate buffer. Blood glucose levels, glucose tolerance, insulin sensitivity, and plasma insulin levels were measured during the 6-week period. A. Fasting blood glucose levels. B and C. Glucose tolerance test (GTT) results at week 3 (b) and week 5 (c) after gene delivery.

FIG. 36 illustrates the effects of the β cell-specific expression of mKL on blood glucose levels, glucose tolerance, insulin sensitivity, and plasma insulin levels in STZ-induced diabetic mice. 129S1/Svlm male mice were injected with PBS, rAAV-GFP, or rAAV-mKL respectively. One week after gene delivery, these mice were injected with STZ or citrate buffer. Blood glucose levels, glucose tolerance, insulin sensitivity, and plasma insulin levels were measured during the 6-week period. A and B. Area under the curve for GTT results at week 3 (a) and week 5 (b) after gene delivery. C. Original readings of insulin sensitivity test (IST) results at week 6 after gene delivery.

FIG. 37 illustrates the effects of the β cell-specific expression of mKL on blood glucose levels, glucose tolerance, insulin sensitivity, and plasma insulin levels in STZ-induced diabetic mice. 129S1/Svlm male mice were injected with PBS, rAAV-GFP, or rAAV-mKL respectively. One week after gene delivery, these mice were injected with STZ or citrate buffer. Blood glucose levels, glucose tolerance, insulin sensitivity, and plasma insulin levels were measured during the 6-week period. A. Normalized blood glucose levels of IST results at week 6 after gene delivery. B. Plasma insulin levels in mice at week 6 after gene delivery. C. Urine glucose levels. Data=mean±SEM. n=4-6 animals/group. *p<005, **p<0.01, ***p<0.001 vs the control (treated with PBS-citrate buffer) group; ⁺⁺p<0.01, ⁺⁺⁺p<0.001 vs the PBS-STZ-treated group.

FIG. 38 illustrates the effects of the β cell-specific expression of mKL on insulin storage and apoptosis in pancreatic islets of mice challenged with STZ. 129S1/Svlm male mice were injected with PBS, rAAV-GFP, or rAAV-mKL. One week after gene delivery, these mice were injected with STZ or citrate buffer. Animals were sacrificed 6 weeks after the rAAV injections. Representative images of Klotho staining (brown color) in cross-sections of mouse pancreatic islets.

FIG. 39 illustrates representative images of insulin staining (brown color) of cross-sections of islets in the mice of FIG. 38.

FIG. 40 illustrates representative images of TUNEL staining (blue color) in pancreatic islets in the mice of FIG. 38.

FIG. 41 illustrates the effects of the β cell-specific expression of mKL on insulin storage and apoptosis in pancreatic islets of mice (of FIG. 38) challenged with STZ. A: Semi-quantification of Klotho staining in pancreatic islets. B: Semi-quantification of insulin staining in pancreatic islets. C: The percentage of cells with positive insulin-staining in pancreatic islets. D: The number of TUNEL-positive apoptotic cells in pancreatic islets. Data=mean±SEM. n=4-6 animals/group. **p<0.01, ***p<0.001 vs the control group; ⁺⁺⁺p<0.001 vs the PBS-STZ-treated group.

FIG. 42 shows the effects of expression of mKL on basal and STZ-induced and TNFα-induced apoptosis, and cell adhesion to collagen IV in MIN6 β cells. MIN6 β cells were transfected with plasmid DNA including pmKL, pGFP or vehicles (transfection agent alone) for 48 hours. A: Western blot analysis of Klotho protein expression. B: Quantification of full length Klotho protein expression. C: Quantification of short form Klotho protein expression. Results were standardized to β-actin and then expressed as fold changes vs the control group (transfection reagent alone). n=4; **p<0.01, ***p<0.001 vs the control group.

FIG. 43 shows the effects of expression of mKL on basal and STZ-induced and TNFα-induced apoptosis, and cell adhesion to collagen IV in MIN6 β cells of FIG. 42. Apoptotic nuclear change (pointed by arrows, brown color) detected by TUNEL staining in MIN6 β cells. Transfected MIN6 β cells were seeded on collagen IV-coated 6-well plates and then incubated with or without STZ or TNFα for 24 hours.

FIG. 44 shows the effects of expression of mKL on basal and STZ-induced and TNFα-induced apoptosis, and cell adhesion to collagen IV in MIN6 β cells of FIG. 42. A: The percentage of apoptotic cells. Data=means±SEM. n=4. *p<0.05, ***p<0.001 vs the control group; ⁺p<0.05 vs the control-STZ group. ^{̂̂̂}p<0.001 vs the control-TNFα group. B: Quantification of cell adhesion. n=4. **p<0.01, ***p<0.001 vs the control-BSA group; ⁺⁺⁺p<0.001 vs the control-collagen IV group.

FIG. 45 shows the effects of expression of mKL on basal and STZ-induced and TNFα-induced apoptosis, and cell adhesion to collagen IV in MIN6 β cells of FIG. 42. Phase contrast images of transfected MIN6 β cells cultured in dishes coated with 1% BSA or collagen IV (5 μg/mL) for 3 hours.

FIG. 46 illustrates that expression of mKL increased phosphorylations of FAK and Akt and decreased Caspase 3 cleavage in MIN6 β cells. Transfected cells were seeded on collagen IV-coated 6-well plates and then incubated with 1 mM STZ for 24 hours. Cells were lysed with Ripa buffer. A. Western blot analysis of phosphorylated FAK (Tyr: 397; upper panel) and FAK protein (middle panel) in cell lysates. B. Quantification of phosphorylation of FAK. Results were standardized to FAK protein levels and then expressed as fold changes vs the control group. C. Western blot analysis of phosphorylated Akt (Ser: 473; upper panel) and Akt protein (lower panel).

FIG. 47 illustrates that expression of mKL increased phosphorylations of FAK and Akt and decreased Caspase 3 cleavage in MIN6 β cells. A. Quantification of phosphorylation of Akt. Results were standardized to Akt protein levels and then expressed as fold changes vs the control group. B. Western blot analysis of cleaved Caspase 3 in cell lysates. C. Quantification of Caspase 3 cleavage. Results were standardized to total Caspase 3 and then expressed as fold changes vs the control group. n=4. *p<0.05, **p<0.01, ***p<0.001 vs the control vehicle; ⁺⁺p<0.01, ⁺⁺⁺p<0.001 vs the control-1 mM STZ group.

FIG. 48 shows that blocking of integrin β1 abolished the protective effect of Klotho on apoptosis and the promoting effect of Klotho on cell adhesion in MIN6 β cells. Apoptotic nuclear change (pointed by arrows, brown color) detected by TUNEL staining in MIN6 β cells. Transfected MIN6 cells were seeded on collagen IV-coated 6-well plates and then incubated with or without blocking antibody for 24 hours.

FIG. 49 shows that blocking of integrin β1 abolished the protective effect of Klotho on apoptosis and the promoting effect of Klotho on cell adhesion in MIN6 β cells of FIG. 48. A. The percentage of apoptotic cells. B. Quantification of cell adhesion. n=4. *p<0.05, ***p<0.001 vs the mock-transfection group; ⁺⁺p<0.01, ⁺⁺⁺p<0.001 vs the pmKL group.

FIG. 50 shows that blocking of integrin β1 abolished the protective effect of Klotho on apoptosis and the promoting effect of Klotho on cell adhesion in MIN6 β cells of FIG. 48. Phase contrast images of adherent MIN6 β cells. Transfected cells were preincubated with integrin β1 blocking antibody or isotype control for 1 hour and then seeded on 6-well plates coated with collagen IV for 3 hours.

FIG. 51 shows that blocking of integrin β1 abolished the promoting effects of Klotho on phosphorylations of FAK and Akt and the inhibiting effects of Klotho on Caspase 3 cleavage in MIN6 β cells. Transfected cells were seeded on collagen IV-coated 6-well plates and then incubated with or without blocking antibody for 24 hours. Cells were lysed with Ripa buffer. 51a: Western blot analysis of phosphorylated FAK (Tyr: 397; upper panel) and FAK protein levels (middle panel) in cell lysates. Quantification of Phosphorylation of FAK (lower figure). Results were standardized to FAK protein level and then expressed as fold changes vs the control mock transfection group. 51b: Western Blot analysis of phosphorylated Akt (Ser: 473; upper panel) and Akt protein (middle panel) levels. Quantification of phosphorylation of Akt (lower figure). Results were standardized to Akt protein level and then expressed as fold changes vs the control mock transfection group.

FIG. 52 shows that blocking of integrin β1 abolished the promoting effects of Klotho on phosphorylations of FAK and Akt and the inhibiting effects of Klotho on Caspase 3 cleavage in MIN6 β cells of FIG. 51. A. Western Blot analysis of cleaved Caspase 3. Quantification of Caspase 3 cleavage (lower figure). Results were standardized to total Caspase and then expressed as fold changes vs the control mock transfection group 3. B. Co-immunoprecipitation of Klotho with integrin β1. Transfected cells were lysed with Ripa buffer. Integrin β1 was immunoprecipitated with antibody against integrin β1. Klotho in the precipitate was detected with antibody against Klotho (lower two panels). The blot was re-probed with antibody against integrin β1 after stripping (upper panel).

FIG. 53 shows body weights and expressions of GFP or FLAG-tag in pancreatic islets of Langerhans. A. Body weights of 129Sv mice treated with STZ. KL^{+/-} and litter mate wild type male mice were injected with STZ or citrate buffer. B. Body weights for 12951/Svlm mice. 12951/Svlm male mice were injected with PBS, rAAV-GFP, or rAAV-mKL. One week after gene delivery, these mice were injected with STZ or citrate buffer.

FIG. 54 shows body weights and expressions of GFP or FLAG-tag in pancreatic islets of Langerhans. Representative images of GFP staining (brown color) in cross-sections of pancreatic islets. Six weeks after the initial rAAV injections, animals were sacrificed and mouse pancreases were collected. GFP protein was detected using antibody against GFP on cross section of mouse pancreas.

FIG. 55 shows body weights and expressions of GFP or FLAG-tag in pancreatic islets of Langerhans. Representative images of FLA-tag staining (brown color) in cross-sections of pancreatic islets. The 3′ end of mouse KL gene was coupled with Flag-tag gene in the construct of rAAV-mKL. FLAG-tag was detected using antibody against FLAG-tag on cross section of mouse pancreas.

### DETAILED DESCRIPTION

Before explaining at least one embodiment of the presently disclosed inventive concepts in more detail by way of exemplary description, examples, and results, it is to be understood that the presently disclosed inventive concepts is not limited in its application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances features which are well known to persons of ordinary skill in the art have not been described in detail to avoid unnecessary complication of the description.

Unless otherwise defined herein, scientific and technical terms used in connection with the presently disclosed inventive concepts shall have the meanings that are commonly understood by those having ordinary skill in the art. Further, unless otherwise required by context, singular terms shall include pluralities and plural terms shall include the singular.

All of the compositions and methods of their application disclosed herein can be made and executed without undue experimentation in light of the present disclosure. While the compositions and methods of the presently disclosed inventive concepts have been described in terms of particular embodiments, it will be apparent to those of skill in the art that variations may be applied to the compositions and/or methods and in the steps or in the sequence of steps of the method described herein without departing from the concept, spirit and scope of the presently disclosed inventive concepts. All such similar substitutes and modifications apparent to those skilled in the art are deemed to be within the spirit, scope and concept of the presently disclosed inventive concepts.

All patents, published patent applications, and non-patent publications mentioned in the specification are indicative of the level of skill of those skilled in the art to which this presently disclosed inventive concepts pertain. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” Throughout this application, the term “about” is used to indicate that a value includes the inherent variation of error for the composition, the method used to administer the composition, or the variation that exists among the study subjects. The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, or at least 85% of the time, or at least 90% of the time, or at least 95% of the time, or at least 98% of the time.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

The term “protein product” as used herein includes natural, recombinant, and/or synthetic proteins, biologically active protein variants (including insertion, substitution and deletion variants), and/or chemically modified derivatives thereof. Included are protein products that are substantially homologous to the human and/or animal protein products.

The term “biologically active” as used herein means that the protein product demonstrates similar properties, but not necessarily all of the same properties, and not necessarily to the same degree, as the natural human and/or animal protein products. Further, by “biologically active” is meant the ability to modify the physiological system of an organism without reference to how the active agent has its physiological effects.

As used herein, the terms “pure” or “substantially pure” mean an object species is the predominant species present (i.e., on a molar basis it is more abundant than any other component in the composition thereof), and a “substantially purified fraction” may be a composition wherein the object species comprises at least about 50 percent (on a molar basis) of all macromolecular species present. Generally, a substantially pure composition will comprise more than about 80 percent of all macromolecular species present in the composition, such as more than about 85%, 90%, 95%, and 99%. In certain embodiments, the object species is purified to essential homogeneity (contaminant species cannot be detected in the composition by conventional detection methods), wherein the composition consists essentially of a single macromolecular species. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The term “substantially homologous” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 95%, or in excess of 96%, or in excess of 97%, or in excess of 98%, or in excess of 99%. For example, in regard to peptides or polypeptides, the percentage of homology as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3: 266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

The terms “polynucleotide sequence” or “nucleic acid,” as used herein, include any polynucleotide sequence which encodes a respective protein product including polynucleotides in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand. The polynucleotide sequence encoding a klotho protein, or encoding a therapeutically-effective fragment of a klotho protein can be the same or substantially the same as the coding sequence of the endogenous klotho coding sequence as long as it encodes a biologically active klotho protein product. Similarly, the polynucleotide sequence can be the same or substantially the same as the klotho gene of a non-human species as long as it encodes a biologically active klotho protein product. Further, the klotho protein, or therapeutically-effective fragment of a klotho protein may be expressed using polynucleotide sequence(s) which differ in codon usage due to the degeneracies of the genetic code or allelic variations.

The terms “infection,” “transduction,” and “transfection” are used interchangeably herein and mean introduction of a gene, nucleic acid, or polynucleotide sequence into cells such that the encoded protein product is expressed. The polynucleotides of the presently disclosed inventive concepts may comprise additional sequences, such as additional coding sequences within the same transcription unit, controlling elements such as promoters, ribosome binding sites, transcription terminators, polyadenylation sites, additional transcription units under control of the same or different promoters, sequences that permit cloning, expression, homologous recombination, and transformation of a host cell, and any such construct as may be desirable to provide embodiments of the presently disclosed inventive concepts.

As used herein, the term “subject” or “patient” refers to a warm blooded animal, particularly a mammal, which is afflicted with a condition or disease described herein. It is understood that guinea pigs, dogs, cats, rats, mice, horses, goats, cattle, sheep, zoo animals, livestock, monkeys, primates, humans, and any other animals with mammary tissue are examples of animals within the scope of the meaning of the term.

“Treatment” refers to therapeutic treatments. “Prevention” refers to prophylactic or preventative treatment measures. The term “treating” refers to administering the composition to a patient or subject for therapeutic purposes. “Treatment” refers to both therapeutic treatment and prophylactic or preventative measures. Those in need of treatment include, but are not limited to, individuals already having a particular condition or disease as well as individuals who are at risk of acquiring a particular condition or disease (e.g., those needing prophylactic/preventative measures). The term “treating” refers to administering an agent to a subject for therapeutic and/or prophylactic/preventative purposes. The term “treat” or “treatment” encompasses the complete range of therapeutically positive effects associated with pharmaceutical medication including reduction of, alleviation of, and relief from the symptoms or illness which affect the subject. When treating diabetes or a diabetes-related condition, treatment includes the administration of a vector to the subject via gene therapy to lower insulin levels, improve glucose tolerance, increase insulin sensitivity, reduce insulin resistance, normalize the blood glucose level, and/or preserve beta cell mass, for example in the subject.

A “therapeutic composition” or “pharmaceutical composition” refers in certain embodiments to a composition comprising a nucleic acid encoding a klotho protein or a nucleic acid encoding a therapeutically-effective fragment of a klotho protein that may be administered to bring about a therapeutic effect as described elsewhere herein.

The term “effective amount” refers in certain embodiments to an amount of a nucleic acid encoding a klotho protein or a nucleic acid encoding a therapeutically-effective fragment of a klotho protein that is sufficient to exhibit a detectable therapeutic effect without undue adverse side effects (such as toxicity, irritation, and allergic response) commensurate with a reasonable benefit/risk ratio when used in the manner of the presently disclosed inventive concepts. The therapeutic effect may include, for example but not by way of limitation, treating a diabetic condition or diabetes-related condition or other disease or condition described herein. The effective amount for a subject will depend upon the type of subject, the subject's size and health, the type and severity of the diabetic condition to be treated, the method of administration, the duration of treatment, the nature of concurrent therapy (if any), the specific formulations employed, and the like. The terms “administration” and “administering” as used herein will be understood to include administration to the subject by any suitable method, such as for example but not by way of limitation, intravenous administration.

A “therapeutically effective amount” of a nucleic acid encoding a klotho protein or a nucleic acid encoding a therapeutically-effective fragment of a klotho protein refers to an amount which is effective in controlling, reducing, or inhibiting a disease or condition described herein. The term “controlling” is intended to refer to all processes wherein there may be a slowing, interrupting, arresting, or stopping of the progression of the disease or condition and does not necessarily indicate a total elimination of the symptoms of the disease or condition.

The term “therapeutically effective amount” is further meant to define an amount resulting in the improvement of any parameters or clinical symptoms characteristic of a particular disease or condition. The actual dose will vary with the subject's overall condition, the seriousness of the symptoms, and counter indications. As used herein, the term “therapeutically effective amount” also means the total amount of each active component of the pharmaceutical composition or method that is sufficient to show a meaningful benefit. When applied to an individual active ingredient administered alone, the term refers to that ingredient alone. When applied to a combination, the term refers to combined amounts of the active ingredients that result in the therapeutic effect, whether administered in combination, serially, and/or simultaneously.

The term “gene therapy” as used herein means genetic modification of cells by the introduction of exogenous DNA or RNA into these cells for the purpose of expressing or replicating one or more peptides, polypeptides, proteins, oligonucleotides, or polynucleotides in vivo for the treatment or prevention of disease or deficiencies in humans or animals. Gene therapy is generally disclosed in U.S. Pat. No. 5,399,346. Any suitable route of administration of the nucleic acid or protein may be employed for providing a subject with pharmaceutical compositions of the presently disclosed inventive concepts. For example, parenteral (subcutaneous, intramuscular, intravenous, transdermal) and like forms of administration may be employed. Dosage formulations include injections, implants, or other known and effective gene therapy delivery methods.

Genetic delivery vehicles (vectors) for the nucleic acids encoding a protein product of the presently disclosed inventive concepts include any vector suitable for promoting expression of the klotho protein product and may comprise an operatively attached promoter sequence which is specific for pancreatic beta cells. Such gene delivery vehicles are well known in the art to persons having ordinary skill in the art; thus, their detailed description is not deemed necessary herein. For example, a nucleic acid encoding a protein product of the presently disclosed inventive concepts may be contained in adeno-associated virus vectors (e.g., as disclosed in U.S. Pat. Nos. 5,139,941, 5,436,146, and 5,622,856), an attenuated or gutless adenoviral vectors, (e.g., as disclosed in Morsy, M. A. and Caskey, C. T., Mol. Med. Today 5:18-24, 1999 and U.S. Pat. No. 5,935,935), lentiviral vectors (such as are disclosed in U.S. Pat. Nos. 5,665,577; 5,994,136; and 6,013,516), plasmids or synthetic (non-viral) vectors (such as disclosed in U.S. Pat. Nos. 4,394,448 and 5,676,954), and/or nanoparticles (such as disclosed, for example, in U.S. Pat. Nos. 6,217,912; 7,514,098; and 8,323,618). Alternative viral vectors include, but are not limited to, retroviral vectors (such as are disclosed in U.S. Pat. Nos. 5,672,510; 5,707,865; and 5,817,491), herpes virus vectors (such as are disclosed in U.S. Pat. No. 5,288,641), and sindbis virus vectors and papilloma virus vectors (such as are disclosed in EP 820 773). The vectors may be either monocistronic, bicistronic, or multicistronic.

An adenoviral vector may include essentially the complete adenoviral genome (Shenk, et al., Curr. Top. Microbiol. Immunol., 111:1-39, 1984). Alternatively, the adenoviral vector may be a modified adenoviral vector in which at least a portion of the adenoviral genome has been deleted. Adenoviral vectors may be produced according to He, et al. (PNAS 95:2590-2514, 1998; Chartier, et al., J. Virol. 70:48054810, 1996 and Hitt, et al., Methods in Molecular Genetics, 7:13-30, 1995). Methods of transferring genes into cells using adenoviral vectors have been described in PCT/US95/15947. A number of adenoviral vectors have been developed for the transduction of genes into cells (Berkner, et al., BioTechniques 6:616-629, 1988). Constitutive high level expression of the transduced gene products has been achieved. These vectors have the inherent advantage over the retroviral vectors in not requiring replicating cells for infection, making them suitable vectors for somatic gene therapy (Mulligan, R. C., Science 260:926-932, 1993). The feasibility for transducing genes associated with glucose metabolism, using adenovirus-mediated transfer in primary rat hepatocytes and myoblast in culture, has been described (Baque, et al., Biochem. J. 304 (Pt 3):1009-1014, 1994; Gomez-Foix, et al., J. Biol. Chem. 267:25129-25134, 1992).

Turning now to the presently disclosed inventive concepts, compositions comprising a klotho protein (and/or a therapeutically effective fragment, variant, and/or derivative thereof) are provided, along with compositions comprising a nucleotide sequence encoding the klotho protein/fragment/variant/derivative. Also provided are methods of producing these compositions, along with methods of use thereof. In one embodiment, the compositions are utilized for treatment of diabetes or diabetes-related diseases or conditions in a subject. In the method, any of the compositions disclosed herein is administered to a subject having diabetes or a diabetes-related disease or condition, such as but not limited to, diabetes mellitus (T1DM and T2DM) and hyperinsulenima (pre-diabetes). Diabetes-related diseases or conditions include, but are not limited to: obesity; peripheral arterial disease (PAD) of the arms, legs, and feet; foot ulcers; hypertension; diabetic neuropathy; diabetic retinopathy; diabetic kidney disease; ketoacidosis; and hyperosmolar hyperglycemic nonketotic syndrome (HHNS). As noted, a particular treatment method is by gene therapy, for example via adeno-associated virus, lentivirus, plasmid, or nanoparticle. In certain embodiments, the treatment results in a reduction in the occurrence and/or severity of diabetes or diabetes-related diseases or conditions in a subject.

In certain embodiments, the treatments of the presently disclosed inventive concepts use a pharmaceutical composition comprising a vector that expresses the desired klotho protein/fragment/variant/derivative in vivo under appropriate or suitable conditions or in a suitable host cell. The pharmaceutical compositions can comprise, consist essentially of, or consist of one or more vectors, e.g., expression vectors, such as in vivo expression vectors, comprising, consisting essentially of, or consisting of and expressing a nucleic acid encoding a klotho protein/fragment/variant/derivative in combination with a pharmaceutically acceptable carrier, excipient, and/or vehicle. In certain embodiments, the vector comprises, consists essentially of, or consists of and expresses at least one nucleic acid encoding a klotho protein/fragment/variant/derivative, in a pharmaceutically acceptable carrier, excipient, and/or vehicle. Thus, according to an embodiment of the presently disclosed inventive concepts, the other vector(s) in the composition comprises a nucleic acid encoding a klotho protein/fragment/variant/derivative.

The pharmaceutically acceptable carrier, vehicle, and/or excipient facilitates transfection and/or improves preservation of the vector. Any pharmaceutically acceptable carriers, vehicles, and excipients known in the art or otherwise contemplated herein may be utilized in accordance with the presently disclosed inventive concepts. For example but not by way of limitation, a pharmaceutically acceptable carrier, vehicle, or excipient can be water, a 0.9% NaCl (e.g., saline) solution, or a phosphate buffer. Other pharmaceutically acceptable carriers, vehicles, and excipients that can be used in the methods of the presently disclosed inventive concepts include, but are not limited to, poly(L-glutamate) or polyvinylpyrrolidone. The pharmaceutically acceptable carrier, vehicle, or excipient may be any compound or combination of compounds facilitating the administration of the vector, increasing the level of expression, and/or increasing the duration of expression.

Doses and dose volumes are discussed herein in the general description and can also be determined by the skilled artisan from this disclosure read in conjunction with the knowledge in the art, without any undue experimentation. For example, the dose volumes can be between about 0.1 and about 2 ml, such as between about 0.2 and about 1 ml. The therapeutic and/or pharmaceutical compositions, in non-limiting embodiments, contain viral particles per dose in a range of, for example, from about 10⁴ to about 10¹¹ particles, from about 10⁵ to about 10¹⁰ particles, or from about 10⁶ to about 10⁹ particles. The presently disclosed inventive concepts contemplate at least one administration to a subject of an efficient amount of the therapeutic composition made according to the presently disclosed inventive concepts. This administration may be via various routes including, but not limited to, intramuscular (IM), subcutaneous (SC), intravascular (IV), and/or intrapancreatic injection.

By way of illustration, klotho variants, derivatives, and the like that are encompassed by the proteins/nucleic acids of the presently disclosed inventive concepts include, but are not limited to, klotho variants, derivatives, and the like that are encoded by nucleotide sequences that are not exactly the same as the nucleotide sequences disclosed herein, but wherein the changes in the nucleotide sequences do not change the encoded amino acid sequence, or merely result in conservative substitutions of amino acid residues, deletion and/or addition of one or a few amino acids, substitution of amino acid residues by amino acid analogs that do not significantly affect the properties of the encoded polypeptides, and the like. Examples of conservative amino acid substitutions include, but are not limited to, glycine/alanine substitutions; valine/isoleucine/leucine substitutions; asparagine/glutamine substitutions; aspartic acid/glutamic acid substitutions; serine/threonine/methionine substitutions; lysine/arginine substitutions; and phenylalanine/tyrosine/tryptophan substitutions. Other types of substitutions, variations, additions, deletions and derivatives that result in functional klotho derivatives and homologs, as described above, are also encompassed by the presently disclosed inventive concepts, and one of skill in the art would readily know how to make, identify, or select such variants or derivatives, and how to test for klotho activity of those variants or derivatives.

One of ordinary skill in the art may optimize the expression of the klotho polypeptides of the presently disclosed inventive concepts to improve expression by any methods known in the art, including but not limited to, by removing cryptic splice sites, by adapting the codon usage by introducing a Kozak consensus sequence before the start codon, by changing the codon usage, or any combination thereof.

Specific but non-limiting examples of klotho-encoding nucleic acids suitable for implementing some embodiments of the presently disclosed inventive concepts are described herein. In particular, for example, SEQ ID NO:1, SEQ ID NO:2, and SEQ ID NO:3 are human klotho-encoding nucleic acids, and SEQ ID NO:4 and SEQ ID NO:5 are mouse klotho-encoding nucleic acids. The listed nucleic acid sequences (SEQ ID NOS: 1-5), and the polypeptides encoded by said nucleic acids, are fully described in U.S. Published Patent Application 2003/0176348 (based on PCT/JP1997/004585), and are expressly incorporated herein by reference in their entireties.

Any pancreatic beta-cell-specific promoter sequence may be used with the Klotho-encoding nucleic acids, for example, an allogenic sequence, such as but not limited to, a human insulin promoter for human treatments and mouse insulin promoter for mouse treatments. Non-limiting examples of insulin promoter sequences which can be used herein are discussed in “Comparative Analysis of Insulin Gene Promoters”, C. Hay and K. Docherty, Diabetes, (2006) 55:3201-3213, 2006; U.S. Published Applications 2004/0191901, 2005/0059620, 2008/0320609, and 2012/023371; and U.S. Pat. No. 6,187,991 (which shows rat insulin promoter therein as SEQ ID NO: 2).

In one embodiment, the presently disclosed inventive concepts comprise a human or non-human nucleic acid variant having identity or homology of at least 80%, at least 85%, at least 90%, at least 91%, at least 92%, at least 93%, at least 94%, at least 95%, at least 96%, at least 97%, at least 98%, or at least 99% to at least one or more of SEQ ID NO:1, SEQ ID NO:2, SEQ ID NO:3, SEQ ID NO:4, SEQ ID NO:5, and SEQ ID NO:6. In another embodiment, the presently disclosed inventive concepts comprise a human or non-human polypeptide variant having identity or homology of at least 80%, at least 85%, at least 90%, at least 91%, at least 92%, at least 93%, at least 94%, at least 95%, at least 96%, at least 97%, at least 98%, or at least 99% to a polypeptide encoded by at least one of SEQ ID NO:1, SEQ ID NO:2, SEQ ID NO:3, SEQ ID NO:4, SEQ ID NO:5, and SEQ ID NO:6, or to a polypeptide comprising at least one of SEQ ID NO:7, SEQ ID NO:8, SEQ ID NO:9, SEQ ID NO:10, and SEQ ID NO:11 disclosed herein.

In some embodiments, the DNA encoding klotho protein, fragment, variant, or derivative is a DNA which hybridizes with a DNA described above under stringent conditions. By “DNA which hybridizes under stringent conditions” is meant DNA obtained by colony hybridization, plaque hybridization, or Southern blot hybridization using DNA encoding klotho protein, specifically including DNA identified after hybridization, using a filter on which colony- or plaque-derived DNA has been immobilized in the presence of 0.7 to 1.0 M NaCl at 65° C. and washing the resulting filter using 0.1× to 2×SSC solutions (the composition of 1×SSC solution comprises 150 mM sodium chloride and 15 mM sodium citrate) at 65° C. Hybridization can be carried out according to a method described, for example, in Molecular Cloning, A Laboratory Manual, the 2nd edition (Sambrook, Fritsch, & Maniatis eds., Cold Spring Harbor Laboratory Press, 1989).

In some embodiments, it is desired that the nucleic acid administered to a subject encode an allogeneic (from the same species as the subject) klotho protein/fragment/variant/derivative, as opposed to a heterologous klotho protein/fragment/variant/derivative. For example, for treating diabetes in humans, a DNA encoding human klotho protein (e.g., nucleic acid SEQ ID NOS: 1, 2, or 3 disclosed herein) may be used in some embodiments, and for treating diabetes in a mouse, a DNA encoding murine klotho protein (e.g., nucleic acid SEQ ID NOS: 4 or 5 disclosed herein) may be used in some embodiments.

T2DM generally progresses through several phases from pre-diabetes to full-blown insulin dependence. As elevated glucose levels occur in the body, it will place a higher demand on insulin secreting pancreatic beta-cells to produce insulin and restore glucose homeostasis (or alleviate postprandial spikes). This increased demand to fold insulin leads to endoplasmic reticulum stress (ERS) in the beta cells. ERS can also result in phosphorylation of the insulin receptor, which attenuates insulin efficacy, causing insulin resistance and, as a result, further increases the demand for insulin production. As this ERS in the beta cells continues, it will eventually lead to a loss in beta cell mass, which has been observed in autopsies of T2DM individuals. Since beta cells do not appear to be regenerated in the pancreas, this loss in beta cell mass leads to the fully insulin-dependent phenotype of later stage T2DM patients.

Preservation of pancreatic beta cells thus maintains beta cell mass due to amelioration of ERS. The latter serves to prevent one of the most deleterious outcomes in T2DM, which is loss of beta cell mass. In regard to T1DM, amelioration of beta cell loss during the early stages of the disease helps preserve beta cell mass while treatments against beta cell-killing autoimmunity of T1DM are mounted.

Therefore, in at least one embodiment, the presently disclosed inventive concepts include a method of preserving beta cell mass in a subject suffering from diabetes or a diabetic-related condition by administering at least one of the pharmaceutical compositions disclosed or otherwise contemplated herein to the subject. In particular embodiments, the pharmaceutical composition comprises at least one of a klotho protein, fragment, variant, derivative and/or a nucleic acid encoding the klotho protein, fragment, variant, or derivative. In certain embodiments, the nucleic acid is disposed in a vector, and the nucleic acid may be operatively connected to a pancreatic beta cell-specific promoter as noted above. The preservation of beta cell mass in the subject can be shown by an increase or stabilization in the amount of insulin production and/or C-peptide production in the subject (as measured by blood or urine tests). In one suitable assay, beta cell mass is indirectly calculated by determining the ratio of C-peptide-to-glucose following oral glucose ingestion, particularly as measured 15 minutes after glucose ingestion (Meier et al., Diabetes 2009; 58:1595-1603). Alternatively, beta cell mass preservation can be indirectly calculated by using the Homeostasis model assessment (HOMA) index (Matthews et al., Diabetologia 1985; 28: 412-419). Additionally, in at least one embodiment, the administration of a nucleic acid encoding a klotho protein, fragment, variant, or derivative to a subject having diabetes or diabetes-related condition results in a blood hemoglobin A1C value which is less than about 7% in the subject.

A therapeutically effective amount of the nucleic acid used in the treatments described herein can be readily determined by the attending diagnostician, as one of ordinary skill in the art, by the use of conventional techniques and by observing results obtained under analogous circumstances. In determining the therapeutically effective dose, a number of factors are considered by the attending diagnostician, including, but not limited to: the species of mammal; its size, age, and general health; the specific disease or condition involved; the degree of or involvement or the severity of the disease or condition; the response of the individual subject; the particular compound administered; the mode of administration; the bioavailability characteristic of the preparation administered; the dose regimen selected; the use of concomitant medication; and other relevant circumstances. A therapeutically effective amount of a nucleic acid encoding a klotho protein, fragment, variant, or derivative thereof also refers to an amount of a nucleic acid which is effective in controlling or reducing the disease or condition. The amount of the nucleic acid present in the pharmaceutical composition of the presently disclosed inventive concepts will depend upon the nature and severity of the diabetic condition being treated, and on the nature of prior treatments which the patient has undergone. Ultimately, the attending physician will decide the amount of nucleic acid with which to treat each individual patient.

In at least one embodiment, the presently disclosed inventive concepts include a method of treating a diabetic or pre-diabetic condition or other diabetes-related condition in a subject in need of such treatment, comprising administering to the subject at least one of the pharmaceutical compositions disclosed or otherwise contemplated herein. In particular embodiments, the pharmaceutical composition may comprise at least one of a klotho protein (or a therapeutically-effective fragment, variant, or derivative thereof) and/or a nucleic acid encoding the klotho protein, fragment, variant, or derivative. In certain embodiments, the nucleic acid is disposed in a vector, and the nucleic acid may be operatively connected to a pancreatic beta cell-specific promoter. In particular non-limiting embodiments, the method may be a treatment which results, in the subject, in at least one of: (1) an increase in insulin sensitivity, (2) an increase in insulin storage, (3) an increase in blood insulin, (4) a decrease in insulin resistance, (5) a decrease in blood glucose levels, (6) a stabilization or increase in C-peptide production, e.g., as indicated by a measurement of C-peptide production, and (7) a hemoglobin A1C value less than about 7%. Non-limiting examples of diabetic conditions that may be treated include Type I diabetes mellitis or Type II diabetes mellitis, or hyperinsulenima (pre-diabetes). Non-limiting examples of diabetes-related diseases or conditions which can be treated by the method include: obesity, peripheral arterial disease (PAD) of the arms, legs, and feet, foot ulcers, hypertension, diabetic neuropathy, diabetic retinopathy, diabetic kidney disease, ketoacidosis, and hyperosmolar hyperglycemic nonketotic syndrome (HHNS). The methods of the presently disclosed inventive concepts also result in increases in insulin I, insulin II, Pdx-1, PCNA, and LC3 mRNA levels and a decrease in DNAJC3 mRNA levels in pancreatic islets of the subject.

Type 2 diabetes mellitus (T2DM) is a complex pathophysiologic disorder, including insulin resistance and β cell failure. There is no hyperglycemia if the compensatory ability of β cells is intact. Protein expression of an anti-aging gene Klotho (Klotho) is depleted in pancreatic islets in db/db mice, a model of T2DM. The presently disclosed inventive concepts are directed in certain embodiments to novel treatments wherein diabetes (e.g., T2DM) and associated cellular processes and outcomes are reduced or mitigated in subjects who receive the Klotho gene/protein. Here it is shown for the first time that β cell-specific expression of introduced mouse Klotho gene (mKL) in vivo attenuates the development of diabetes in db/db mice. β cell-specific expression of mKL decreased hyperglycemia and enhanced glucose tolerance. The beneficial effects of mKL were associated with significant improvements in T2DM-induced decreases in the number of β cells, insulin storage levels in pancreatic islets, and the glucose-stimulated insulin secretion from pancreatic islets which led to increased blood insulin levels in diabetic mice. In addition, β cell-specific expression of mKL decreased the intracellular superoxide levels, oxidative damage, apoptosis, and DNAJC3 (a marker for endoplasmic reticulum stress) in pancreatic islets. Furthermore, a cell-specific expression of mKL increased expression levels of Pdx-1 (insulin transcription factor), PCNA (a marker of cell proliferation), and LC3 (a marker of autophagy) in pancreatic islets in db/db mice. These results revealed that β cell-specific expression of mKL improved β cell function and attenuated the development of T2DM. Therefore, in vivo expression of the Klotho gene in pancreatic β cells offers a novel therapeutic strategy for β cell dysfunction in subjects having T2DM.

Moreover, apoptosis is the major cause of death of insulin-producing β cells that leads to diabetes, particularly type 1 diabetes mellitus (T1DM). Here, it is shown that half deficiency of Klotho in heterozygous Klotho mutant (KL^{+/-}) mice exacerbated streptozotocin (STZ)-induced diabetes (a model of T1DM), including hyperglycemia, glucose intolerance, diminished islet insulin storage, and increased apoptotic islet cells. Conversely, in such mice given mKL in vivo, β cell-specific expression of mKL prevented STZ-induced diabetes and attenuated β cell apoptosis. In MIN6 β cells, expression of mKL promoted cell adhesion to collagen IV, attenuated apoptosis, enhanced phosphorylations of FAK and Akt, and inhibited Caspase 3 cleavage. Expression of mKL in MIN6 β cells abolished STZ- and TNFα-induced apoptosis, eliminated STZ-induced inhibition of phosphorylations of FAK and Akt, and decreased STZ-induced Caspase 3 cleavage. Furthermore, blocking integrin β1 abolished the promoting effects of Klotho on cell adhesion and phosphorylations of FAK and Akt, and eliminated the protective effects of Klotho against apoptosis and Caspase 3 cleavage, demonstrating that integrin β1 is potentially an important mediator of Klotho-induced protection in MIN6 β cells. Therefore, these results reveal for the first time that Klotho prevented STZ-induced T1DM by inhibiting pancreatic β cell apoptosis. The presently disclosed inventive concepts are thus also directed to a therapeutic method of treating and inhibiting T1DM by administration of any of the compositions disclosed or otherwise contemplated herein.

As noted elsewhere herein, the diabetes or diabetes-relation condition may be treated by administration of a composition comprising a klotho protein or a fragment, variant, or derivative thereof, for example a soluble form of the protein. Klotho proteins, including soluble forms, which may be used in the presently disclosed inventive concepts include but are not limited to SEQ ID NO:7, SEQ ID NO:8, SEQ ID NO:9, SEQ ID NO:10, and SEQ ID NO:11, and effective fragments thereof. The klotho protein, fragment, variant, or derivative may be any suitable klotho protein, fragment, variant, or derivative and may be made, isolated, and purified in any suitable fashion with which one skilled in the art, for example as described in U.S. Published Patent Application 2003/0176348. Specific representative examples of klotho proteins/fragments/variants/derivatives suitable for use in implementing some embodiments of the teachings of the presently disclosed inventive concepts include klotho proteins described in U.S. Published Patent Application 2003/0176348; U.S. Pat. No. 6,579,850; and U.S. applications 2010/0330062, 2012/0178699 (e.g., SEQ ID NOS: 1, 2, and 5, therein); and 2012/0232024 (e.g., SEQ ID NOS: 4 and 5 therein). In some embodiments, a klotho protein used in implementing the teachings of the presently disclosed inventive concepts is a complete, or substantially complete, klotho protein. In some embodiments, the therapeutically-effective fragment of klotho protein, (such as proteins comprising SEQ ID NO:7, SEQ ID NO:8, SEQ ID NO:9, SEQ ID NO:10, or SEQ ID NO:11) may comprise less than 549 amino acids, such as from about 10 to about 548 amino acids. The number of amino acids in the fragment may be in a range of from 10-25 amino acids, 26-50 amino acids, 51-75 amino acids, 76-100 amino acids, 101-125 amino acids, 126-150 amino acids, 151-175 amino acids, 176-200 amino acids, 201-225 amino acids, 226-250 amino acids, 251-275 amino acids, 276-300 amino acids, 301-325 amino acids, 326-350 amino acids, 351-375 amino acids, 376-400 amino acids, 401-425 amino acids, 426-450 amino acids, 451-475 amino acids, 476-500 amino acids, 501-525 amino acids, or 526-548 amino acids, or any range between any integer within any of said ranges, such as from 160 to 260 amino acids.

Although not wishing to be bound by theory, it is believed that in some embodiments, a complete or substantially complete klotho protein or effective fragment thereof that is administered to a subject is modified in the body of a subject (for example, by cleavage of at least one peptide bond, shortening of the N-terminus and/or shortening of the C-terminus) and the thus-modified polypeptide provides the desired effect described herein.

In some embodiments, a klotho protein used in implementing the presently disclosed inventive concepts is a cleaved extracellular portion of a complete klotho protein. In some embodiments, a klotho protein used in implementing the presently disclosed inventive concepts is a portion of a complete klotho protein. Typical such portions of complete klotho proteins are isolated polypeptides such as shown in the referenced patents and patent applications incorporated by reference herein.

In some embodiments, a klotho protein used in implementing the teachings of the presently disclosed inventive concepts is a soluble klotho protein, for example solubilized by removal of one or more amino acid residues from the C-terminus, the N-terminus or both the C-terminus and the N-terminus, as is known to one skilled in the art. Typical such solubilized klotho proteins are isolated polypeptides including an amino acid sequence selected from the group of amino acid sequences represented in U.S. 2003/0176348, U.S. 2012/0178699, and U.S. 2010/0330062, and SEQ ID NOS: 7 and 9 presently listed.

In some embodiments, non-limiting examples of klotho protein/fragment/variant/derivative used in implementing the presently disclosed inventive concepts are isolated polypeptides having a homology of at least 80% or more (e.g., at least 90%, at least 91%, at least 92%, at least 93%, at least 94%, at least 95%, at least 96%, at least 97%, at least 98%, or at least 99% homology) with a polypeptide having the amino acid sequence represented by either of SEQ ID NOS: 1, 2, 5, or 11-15 disclosed in US 2012/0178699, or SEQ ID NOS: 7-11 provided herein.

In some embodiments, a klotho protein, fragment, variant, or derivative administered to a subject may be allogeneic (from the same species as the subject) as opposed to heterologous. For example, for treating cancer in humans, in some embodiments a human klotho protein (e.g., SEQ ID NOS: 1, 2, 5, 11-15 of U.S. 2012/0178699 or homologous proteins as described above) may be used. According to some embodiments of the presently disclosed inventive concepts, the klotho protein is a native protein, a derivative or analog thereof, or an active segment of the native protein. According to some embodiments of the presently disclosed inventive concepts, the klotho protein is a soluble or solubilized form of klotho protein. According to some embodiments, the klotho protein is a PEGylated klotho protein, for example, a protein substantially similar or identical to klotho proteins described herein that has been PEGylated as described elsewhere herein or otherwise known in the art to improve pharmacokinetics or other parameters.

The compositions of the presently disclosed inventive concepts may be administered by any suitable route, for example but not by way of limitation, as described in U.S. Published Patent Application 2003/0176348. Particular non-limiting examples of administration routes include parenteral, intravenous, intradermal, intraperitoneal, intramuscular, subcutaneous, oral, nasal, buccal, sublingual, intra-tracheal, transdermal, transmucosal, and pulmonary routes. The compositions may be administered by continuous release or delivery, using, for example, an infusion pump, continuous infusion, and/or controlled release formulations utilizing polymer, oil or water insoluble matrices. Carriers or excipients known in the art can also be used to facilitate administration of the compositions. Non-limiting examples of carriers and excipients include calcium carbonate, calcium phosphate, various sugars such as lactose, or types of starch, cellulose derivatives, gelatin, vegetable oils, polyethylene glycols, physiologically compatible solvents, and combinations thereof.

According to some embodiments, the compositions of the presently disclosed inventive concepts are administered in extended release form, which is capable of releasing the protein/fragment/nucleic acid over a predetermined release period, such that a clinically effective plasma level of the klotho protein/fragment is maintained for at least 24 hours, such as at least 48 hours, at least 72 hours, at least one week, or at least one month.

Leading up to the work presently disclosed herein it was demonstrated that Klotho mRNA and proteins are expressed in mouse pancreatic islets, and that silencing of Klotho impaired glucose-stimulated insulin secretion in MIN6 β cells. Prior to the present work, it was not known if Klotho expression is altered in pancreatic β cells in subjects with T1DM or T2DM. Further whether Klotho protects β cell function in subjects with T1DM or T2DM had not been investigated prior to the present disclosure. The results provided herein demonstrate that Klotho protects β cells, attenuates hyperglycemia, and enhances glucose tolerance in T2DM and T1DM. Delivery of the Klotho gene or protein (or fragment, variant, or derivative thereof) to a subject in need of such therapy can be used as a novel therapeutic approach for diabetes. Delivery of klotho by AAV or any other suitable means provides a long-term treatment for diabetes. The presently disclosed inventive concepts are also directed to treatments with Klotho protein (or effective fragments, variants, or derivatives thereof), but repeated treatment may be required to be effective.

Other embodiments of the presently disclosed inventive concepts include pharmaceutical compositions that contain a therapeutically-effective or pharmaceutically-effective amount of at least one active substance (i.e., one or more of the nucleic acid, protein, or peptide compositions described herein above) in combination with a pharmaceutically-acceptable carrier. The pharmaceutically acceptable carrier may be a pharmaceutically acceptable solvent, suspending agent, or vehicle for delivering the compositions to the subject. The carrier may be, for example but not by way of limitation, liquid or solid, and the carrier may be selected with the planned manner of administration in mind. Examples of pharmaceutically acceptable carriers that may be utilized in accordance with the presently disclosed inventive concepts include, but are not limited to, polyethylene glycol (PEG), polymers, liposomes, ethanol, DMSO, aqueous buffers, solvents, oils, DPPC, lipids, and combinations thereof.

The pharmaceutical compositions may contain, in addition to the active substance and pharmaceutically-acceptable carrier, one or more additional components, including but not limited to, diluents, fillers, salts, buffers, stabilizers, solubilizers, and other materials well known in the art. Suitable carriers, vehicles, and other components that may be Included in the formulation are described, for example, in Remington: The Science and Practice of Pharmacy, 21^{st} ed. The term “pharmaceutically acceptable” means a non-toxic material that does not interfere with the effectiveness of the biological activity of the active ingredient(s). The characteristics of the carrier will depend on the route of administration.

The pharmaceutical composition of the presently disclosed inventive concepts may be in the form of a liposome in which the active substance is disposed, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

A therapeutically effective amount of an active substance of the presently disclosed inventive concept(s) refers to an amount which is effective in controlling, reducing, and/or inhibiting a diabetic condition or diabetes-related condition as discussed elsewhere herein. The term “controlling” is intended to refer to all processes wherein there may be a slowing, interrupting, arresting, and/or stopping of the progression of the condition and does not necessarily indicate a total elimination of the symptoms.

The term “therapeutically effective amount” is further meant to define an amount resulting in the improvement of any parameters or clinical symptoms characteristic of a diabetic condition or diabetes-related condition. The actual dose will vary with the patient's overall condition, the seriousness of the symptoms, and counter indications. As used herein, the term “therapeutically effective amount” also means the total amount of each active component of the pharmaceutical composition or method that is sufficient to show a meaningful patient benefit, e.g., a reduction of the diabetic condition or diabetes-related condition. When applied to an individual active substance that is administered alone, the term refers to that ingredient alone. When applied to a combination, the term refers to combined amounts of the active substances that result in the therapeutic effect, whether administered in combination, serially, and/or simultaneously.

A therapeutically effective amount of the active substance used in the treatment described herein can be determined by the attending diagnostician, as one skilled in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diabetic condition or diabetes-related condition involved; the degree of or involvement or the severity of the diabetic condition or diabetes-related condition; the response of the individual subject; the particular compound administered; the mode of administration; the bioavailability characteristic of the preparation administered; the dose regimen selected; the use of concomitant medication; and other relevant circumstances. A therapeutically effective amount of an active substance of the presently disclosed inventive concepts also refers to an amount of the active substance which is effective in controlling, improving, or reducing the diabetic condition or diabetes-related condition.

A therapeutically effective amount of a protein or peptide of the presently disclosed inventive concept will generally contain sufficient active substance to deliver from about 0.1 μg/kg to about 100 mg/kg (weight of active substance/body weight of the subject). Particularly, the composition will deliver about 0.5 μg/kg to about 50 mg/kg, and more particularly about 1 μg/kg to about 10 mg/kg.

Practice of the method of the presently disclosed inventive concepts may comprise administering to a subject a therapeutically effective amount of the protein or peptide compound in any suitable systemic and/or local formulation, in an amount effective to deliver the dosages listed above. An effective, particular dosage of the protein or peptide compound for treating the diabetic condition or diabetes-related condition may be, in one embodiment, about 1 μg/kg to about 10 mg/kg of the protein or peptide. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the presently disclosed inventive concepts, the protein or peptide compound is provided in an IV infusion in the range of from about 1 mg/kg to about 10 mg/kg of body weight once a day.

Administration of the protein or peptide compound used in the pharmaceutical composition or to practice the method of the presently disclosed inventive concepts can be carried out in a variety of conventional ways, such as, but not limited to, orally, by inhalation, rectally, or by cutaneous, subcutaneous, intraperitoneal, vaginal, or intravenous injection. Oral formulations may be formulated such that the protein or peptide compound passes through a portion of the digestive system before being released, for example it may not be released until reaching the small intestine, or the colon.

When a therapeutically effective amount of the active substance is administered orally, it may be in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active substance. For example, a dose of about 10 mg to about 1000 mg once or twice a day could be administered orally.

In another embodiment, the peptide compounds of the presently disclosed inventive concepts can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the peptide compound in an aqueous or non-aqueous pharmaceutically acceptable solvent which may also contain suspending agents, sweetening agents, flavoring agents, and preservative agents as are known in the art.

For parenteral administration, for example, the protein or peptide compounds may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When a therapeutically effective amount of the protein or peptide compound is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the protein or peptide compound, an isotonic vehicle such as Sodium Chloride Injection, Ringer's injection, Dextrose Injection, Dextrose and Sodium Chloride Injection, Lactated Ringer's Injection, or other vehicle as known in the art. The pharmaceutical composition of the presently disclosed inventive concept may also contain stabilizers, preservatives, buffers, antioxidants, or other additive(s) known to those of skill in the art.

As noted, particular amounts and modes of administration can be determined by one skilled in the art. One skilled in the art of preparing formulations can readily select the proper form and mode of administration, depending upon the particular characteristics of the peptide compound selected, the infection to be treated, the stage of the infection, and other relevant circumstances using formulation technology known in the art, described, for example, in Remington: The Science and Practice of Pharmacy, 21^{st} ed.

The presently disclosed inventive concepts further include (but are not limited to) a method of treating a diabetic condition or diabetes-related condition by topically applying an amount of the composition sufficient to treat the condition, e.g., about 0.5% to about 10% by weight of the composition. The topical medication may take any number of standard forms such as pastes, gels, creams, and ointments. In one embodiment, a solution of the composition to be administered may be prepared using a solvent known to promote transdermal absorption, such as but not limited to, ethanol or dimethyl sulfoxide (DMSO) with or without other excipients. Particularly, topical administration may be accomplished using a patch either of the reservoir and porous membrane type or of a solid matrix variety.

The amount of the protein or peptide compound in the pharmaceutical composition will depend upon the nature and severity of the condition being treated and on the nature of prior treatments which the patient has undergone. Ultimately, the attending physician will decide the amount of active substance with which to treat each individual patient. Initially, the attending physician may administer low doses of the active substance and observe the patient's response. Larger doses may be administered until the optimal therapeutic effect is obtained for the patient, and at that point the dosage is not increased further. Without wishing to be held to a specific dosage, it is contemplated that the various pharmaceutical compositions used to practice the method of the presently disclosed inventive concepts may contain, but are not limited to, about 0.1 mg to about 100 mg of the active substance per kg body weight per dose.

The duration of an intravenous therapy using the pharmaceutical composition of the presently disclosed inventive concept(s) will vary, depending on the severity of the diabetic condition or diabetes-related condition being treated and the condition and potential idiosyncratic response of each individual patient. It is contemplated that the duration of each application may be in the range of about 1 to about 2 hours and given once about every 12 or 24 hours by continuous intravenous administration.

Additional pharmaceutical methods may be employed to control the duration of action of the protein or peptide compound. Increased half-life and/or controlled release preparations may be achieved through the use of polymers to conjugate, complex with, and/or absorb the active substance described herein. The controlled delivery and/or increased half-life may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The protein or peptide compound may also be ionically or covalently conjugated to the macromolecules described above.

Another possible method useful in controlling the duration of action of the active substance by controlled release preparations and half-life is incorporation of the protein or peptide compound or its functional derivatives into particles of a polymeric material such as polyesters, polyamides, polyamino acids, hydrogels, poly(lactic acid), ethylene vinylacetate copolymers, copolymer micelles of, for example, polyethylene glycol (PEG) and poly(I-aspartamide). The protein or peptide compound may also be ionically or covalently conjugated to the macromolecules described above, particularly PEG of various molecular weights. In certain embodiments the protein or peptide compounds are covalently linked at a suitable functional group such as the N-terminal end thereof to one or more PEG molecules to form a “pegylated” protein or peptide. Examples of PEG molecules that can be used include, but are not limited to, a “mini-PEG™” molecule comprising AEEA and/or AEEEA. AEEA is [2-(2-amino-ethoxy)-ethoxy]-acetic acid (also known as 8-Amino-3,6-Dioxaoctanoic acid), and AEEEA is {2-[2-(2-amino-ethoxy)-ethoxy]-ethoxy}-acetic acid (also known as 11-Amino-3,6,9-Trioxaundecanoic acid). The PEG molecule may have a molecular weight in a range of from about 350 Daltons to about 20,000 Daltons. More particularly, the PEG molecule may have a MW in a range of from about 450 Da to about 15,000 Da. More particularly, the PEG molecule may have a MW in a range of from about 1000 Da to about 12,000 Da. More particularly, the PEG molecule may have a MW in a range of from about 2000 Da to about 10,000 Da. More particularly, the PEG molecule may have a MW in a range of from about 3000 Da to about 8,000 Da.

It is also possible to entrap the protein or peptide compounds in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the protein or peptide composition is to be used as an injectable material, it can be formulated into a conventional injectable carrier. Suitable carriers include biocompatible and pharmaceutically acceptable phosphate buffered saline solutions, which are particularly isotonic.

For reconstitution of a lyophilized product in accordance with the presently disclosed inventive concept(s), one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The protein or peptide compounds of the presently disclosed inventive concepts can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines, and substituted ethanolamines.

In at least one embodiment, the presently disclosed inventive concepts include a method of treating a diabetic condition or a diabetes-related condition in a subject in need of such treatment by administering to the subject a therapeutically-effective amount of a vector comprising a nucleic acid which encodes a klotho protein or a therapeutically-effective portion of a klotho protein, wherein the klotho protein or therapeutically-effective portion of the klotho protein is expressed in vivo in pancreatic beta cells of the subject. In this method the vector may include a promoter operatively-linked to the nucleic acid which encodes the klotho protein or the therapeutically-effective portion of a klotho protein, wherein the promoter is specific for pancreatic beta cells. In this method the vector may be selected from the group consisting of adeno-associated virus, nanoparticles, plasmids, and lentivirus. In this method the nucleic acid which encodes the klotho protein or the therapeutically-effective portion of a klotho protein may be selected from the group consisting of SEQ ID NO:1, SEQ ID NO:2, SEQ ID NO:3, SEQ ID NO:4, SEQ ID NO:5, and SEQ ID NO:6, and nucleic acids which have at least 90% identity to at least one of SEQ ID NO:1, SEQ ID NO:2, SEQ ID NO:3, SEQ ID NO:4, SEQ ID NO:5, SEQ ID NO:6, and which encode the klotho protein or the therapeutically-effective portion of a klotho protein. In this method the diabetic condition may be Type I diabetes mellitis or Type II diabetes mellitis. In this method the diabetes-related condition may be at least one of hyperinsulenima (pre-diabetes), obesity, peripheral arterial disease (PAD) of the arms, legs, and/or feet, foot ulcers, hypertension, diabetic neuropathy, diabetic retinopathy, diabetic kidney disease, ketoacidosis, and hyperosmolar hyperglycemic nonketotic syndrome (HHNS). In this method the treatment may result in at least one of (a) an increase in blood Insulin levels in the subject, (b) an increase in insulin storage levels in the subject, (c) an increase in insulin sensitivity in the subject, (d) a decrease in insulin resistance in the subject, (e) a decrease in average blood glucose levels in the subject, (f) a stabilization or an increase in C-peptide production in the subject, (g) a stabilization of beta cell mass in the subject as indicated by a measurement of C-peptide production in the subject, and (h) a hemoglobin A1C value of less than about 7% in the subject.

In at least one embodiment, the presently disclosed inventive concepts include a composition comprising a therapeutically-effective amount of a vector comprising a nucleic acid which encodes a klotho protein or a therapeutically-effective portion of a klotho protein for use in a method of treating a diabetic condition or a diabetes-related condition in a subject in need of such treatment, the method comprising administering the composition to the subject, wherein the klotho protein or therapeutically-effective portion of the klotho protein is expressed in vivo in pancreatic beta cells of the subject. In this method the vector of the composition may include a promoter operatively-linked to the nucleic acid which encodes the klotho protein or the therapeutically-effective portion of a klotho protein, wherein the promoter is specific for pancreatic beta cells. In this method the vector of the composition may be selected from the group consisting of adeno-associated virus, nanoparticles, plasmids, and lentivirus. In this method the nucleic acid of the vector which encodes the klotho protein or the therapeutically-effective portion of a klotho protein may be selected from the group consisting of SEQ ID NO:1, SEQ ID NO:2, SEQ ID NO:3, SEQ ID NO:4, SEQ ID NO:5, and SEQ ID NO:6, and nucleic acids which have at least 90% identity to at least one of SEQ ID NO:1, SEQ ID NO:2, SEQ ID NO:3, SEQ ID NO:4, SEQ ID NO:5, SEQ ID NO:6, and which encode the klotho protein or the therapeutically-effective portion of a klotho protein. In this method the diabetic condition may be Type I diabetes mellitis or Type II diabetes mellitis. In this method the diabetes-related condition may be at least one of hyperinsulenima (pre-diabetes), obesity, peripheral arterial disease (PAD) of the arms, legs, and/or feet, foot ulcers, hypertension, diabetic neuropathy, diabetic retinopathy, diabetic kidney disease, ketoacidosis, and hyperosmolar hyperglycemic nonketotic syndrome (HHNS). In this method the treatment may result in at least one of (a) an increase in blood Insulin levels in the subject, (b) an increase in insulin storage levels in the subject, (c) an increase in insulin sensitivity in the subject, (d) a decrease in insulin resistance in the subject, (e) a decrease in average blood glucose levels in the subject, (f) a stabilization or an increase in C-peptide production in the subject, (g) a stabilization of beta cell mass in the subject as indicated by a measurement of C-peptide production in the subject, and (h) a hemoglobin A1C value of less than about 7% in the subject.

In at least one embodiment, the presently disclosed inventive concepts include a method of treating a diabetic condition or a diabetes-related condition in a subject in need of such treatment by administering to the subject a therapeutically-effective amount of at least one of a klotho protein and a therapeutically-effective portion of the klotho protein thereby mitigating the diabetic condition or diabetes-related condition in the subject. In this method the klotho protein or therapeutically-effective portion of the klotho protein may be selected from the group consisting of SEQ ID NO:7, SEQ ID NO:8, SEQ ID NO:9, SEQ ID NO:10, and SEQ ID NO:11, and therapeutically-effective portions thereof, and therapeutically-effective proteins which have at least 90% identity to at least one of SEQ ID NO:7, SEQ ID NO:8, SEQ ID NO:9, SEQ ID NO:10, and SEQ ID NO:11. In this method the diabetic condition may be Type I diabetes mellitis or Type II diabetes mellitis. In this method the diabetes-related condition may be at least one of hyperinsulenima (pre-diabetes), obesity, peripheral arterial disease (PAD) of the arms, legs, and/or feet, foot ulcers, hypertension, diabetic neuropathy, diabetic retinopathy, diabetic kidney disease, ketoacidosis, and hyperosmolar hyperglycemic nonketotic syndrome (HHNS). In this method the treatment may result in at least one of (a) an increase in blood insulin levels in the subject, (b) an increase in insulin storage levels in the subject, (c) an increase in insulin sensitivity in the subject, (d) a decrease in insulin resistance in the subject, (e) a decrease in average blood glucose levels in the subject, (f) a stabilization or an increase in C-peptide production in the subject, (g) a stabilization of beta cell mass in the subject as indicated by a measurement of C-peptide production in the subject, and (h) a hemoglobin A1C value of less than about 7% in the subject.

In at least one embodiment, the presently disclosed inventive concepts include a composition comprising a therapeutically-effective amount of a klotho protein or a therapeutically-effective portion of a klotho protein for use in a method of treating a diabetic condition or a diabetes-related condition in a subject in need of such treatment, the method comprising administering the composition to the subject thereby mitigating the diabetic condition or diabetes-related condition in the subject. In this method the klotho protein or therapeutically-effective portion of the klotho protein may be selected from the group consisting of SEQ ID NO:7, SEQ ID NO:8, SEQ ID NO:9, SEQ ID NO:10, and SEQ ID NO:11, and therapeutically-effective portions thereof, and therapeutically-effective proteins which have at least 90% identity to at least one of SEQ ID NO:7, SEQ ID NO:8, SEQ ID NO:9, SEQ ID NO:10, and SEQ ID NO:11. In this method the diabetic condition may be Type I diabetes mellitis or Type II diabetes mellitis. In this method the diabetes-related condition may be at least one of hyperinsulenima (pre-diabetes), obesity, peripheral arterial disease (PAD) of the arms, legs, and/or feet, foot ulcers, hypertension, diabetic neuropathy, diabetic retinopathy, diabetic kidney disease, ketoacidosis, and hyperosmolar hyperglycemic nonketotic syndrome (HHNS). In this method the treatment may result in at least one of (a) an increase in blood insulin levels in the subject, (b) an increase in insulin storage levels in the subject, (c) an increase in insulin sensitivity in the subject, (d) a decrease in insulin resistance in the subject, (e) a decrease in average blood glucose levels in the subject, (f) a stabilization or an increase in C-peptide production in the subject, (g) a stabilization of beta cell mass in the subject as indicated by a measurement of C-peptide production in the subject, and (h) a hemoglobin A1C value of less than about 7% in the subject.

TABLE 1
List of Klotho Sequences and their
Corresponding Sequence Identifiers

| SEQ | |
|---|---|
| ID | |
| NO: | Description |
| 1 | Homo sapiens klotho-encoding nucleic acid from US 2003/ |
| | 0176348 |
| 2 | Homo sapiens klotho-encoding nucleic acid from US 2003/ |
| | 0176348 |
| 3 | Homo sapiens klotho-encoding nucleic acid from US 2003/ |
| | 0176348 |
| 4 | Mus musculus klotho-encoding nucleic acid from US 2003/ |
| | 0176348 |
| 5 | Mus musculus klotho-encoding nucleic acid from US 2003/ |
| | 0176348 |
| 6 | Rat Klotho mRNA sequence (gi\|13786167\|ref\|NM_031336.1\| |
| | Rattus norvegicus Klotho (Kl), mRNA) |
| 7 | Human Secreted Klotho - protein (sp\|Q9UEF7-2\|KLOT_HUMAN |
| | Isoform 2 of Klotho OS = Homo sapiens GN = KL) |
| 8 | Human full length - protein (gi\|24497614\|ref\|NP_004786.2\| klotho |
| | precursor [Homo sapiens]) |
| 9 | Mouse secreted Klotho protein |
| 10 | Mouse full length (gi\|227330584\|ref\|NP_038851.2\| klotho |
| | precursor [Mus musculus]) protein |
| 11 | Rat full length protein (gi\|13786168\|ref\|NP_112626.1\| klotho |
| | precursor [Rattus norvegicus]) |

### EXAMPLES

Examples of the presently disclosed inventive concepts are provided hereinbelow. However it is to be understood that the presently disclosed inventive concepts are not to be limited to the specific experimentation, results and laboratory procedures of the Examples. Rather, the Examples are simply provided as various embodiments of the presently disclosed inventive concepts and are meant to be exemplary, not exhaustive.

### Example 1

### Transfection of Db/Db Mice with Klotho

The db/db (diabetic) mouse was originally derived from an autosomal recessive mutation in db gene, which encodes for leptin receptors. This model resembles key features of human T2DM including peripheral insulin resistance and progressive deterioration of pancreatic β cells. Preliminary work showed that the Klotho level in pancreatic islets is decreased significantly in db/db mice. Further work investigated protection of β cell function and attenuation of the development of diabetes in db/db mice by β cell-specific expression of the Klotho gene.

Methods of Example 1:

Cell culture. Pancreatic insulinoma MIN6 β cells were kindly provided by Dr. Miyazaki and Dr. Steiner. MIN6 cells were cultured and maintained in DMEM containing 25 mM glucose, 10% FBS, 1% penicillin/streptomycin, 2 mM glutamine, and 100 μM β-mercaptoethanol. MIN6 cells of passage less than 20 were used in the experiments. 3T3-L1 preadipocytes, and mouse renal inner medullary collecting duct (mlMCD3) cells were cultured in the above media without β-mercaptoethanol.

Human pancreas. The use of human pancreas was approved by the Institutional Research Board (IRB) at the University of Oklahoma Health Sciences Center. Human pancreases from normal donors (age: 37 to 50; mixed gender) and T2DM donors (age: 42 to 49; mixed gender) were obtained from National Disease Research Interchange, the National Resource Center (Philadelphia, Pa.).

AAV vector construction and recombinant viral production. A plasmid of pAAV2.1-mlNSULIN-nLacZ with 1.13-Kb mouse preproinsulin gene II promoter was kindly provided by Dr. X. Xiao (1). A plasmid of pEFmKLcFT with the full-length mouse Klotho cDNA and C-terminal Flag tag was kindly provided by Dr. M. Kuro-o (University of Texas Southwestern Medical Center). The full length Klotho cDNA with the C-terminal Flag tog (3.1 kb in total) was cloned into AAV serotype-2 (AAV2) (Startagen, La Jolla, Calif., USA). The mouse insulin II promoter was cloned into AAV2 by replacing the original CMV promoter and intron. Insulin II promoter and GFP cDNA (700 bs) were cloned into the AAV2 vector as the control constructs. The constructs of pAAV-mKL and pAAV-GFP were then packaged with pHelper and pAAV-RC to produce recombinant adeno-associated viruses by following the manufactory instruction manual (Startagen, La Jolla, Calif., USA). Recombinant viruses were purified via a method of CsCl gradient as described (2). The titers of recombinant viral genome particles were determined on a Bio-Rad CFX96™ Real-Time PCR Detection Systems with a pair of primers targeted insulin II promoter region (F: 5′-AAATGCTCAGCCAAGGACAA-3′ (SEQ ID NO:12) and R: 5′-GGACTTTGCTGTTTGACCCATT-3′ (SEQ ID NO:13)) and with the method as described (3,4). Hereinafter below, these recombinant viruses will be referred as rAAV-GFP and rAAV-mKL, respectively.

Transfection with plasmid DNA. Plasmid DNA including pAAV-mKL, pAAV-GFP, and pAAV-CMV-mKL were purified with Qiagen Maxi Kit. MIN6 cells, 3T3-L1 preadipocytes, and mlMCD3 cells cultured in 6-well plate were transfected with various plasmid DNA at the concentration of 0.072 μg/mL using Optifect™ reagent according to the manufacturer's protocol, followed by 48-hour incubation in DMEM with 10% FBS at 37° C. in a 5% CO₂ incubator. Phase-contrast images and fluorescence images of cells transfected with pAAV-GFP for 48 hours were collected at equal exposure conditions under Nikon Eclipse Ti microscopy (magnification ×100) with the software NIS-Elements BR 3.0 (Nikon).

Western blotting. Cells (or mouse pancreas) were lysed in Ripa buffer 48 hours after the transfection. The lysates were directly subjected to SDS-PAGE followed by western blotting with antibody against Klotho (AF1819, R&D Systems) as were described previously (5,6). The blot was rinsed and reprobed with antibody against β-actin for the loading control.

Animal study protocol. This study was carried out according to the guidelines of the National Institutes of Health on the Care and Use of Laboratory Animals. This project was approved by the Institutional Animal Care and Use Committee at the University of Oklahoma Health Sciences Center. Eight week old male BKS.Cg-Dock7m+/+Lepr^{db}/J HOM (db/db) mice and BKS.Cg-Dock7m+/+Lepr^{db}/J HET (lean) mice were purchased from Jackson Laboratory (Bar Harbor, Me. USA). All mice were housed in cages at room temperatures (25±1° C.) and were provided with Purina laboratory chow (No. 5001) and tap water ad libitum throughout the experiment. Three groups of db/db mice and 3 groups of lean mice were used (7 to 8 mice/group). Body weight was monitored weekly. Blood glucose was measured weekly from the tail vein blood using a Reli On Ultima glucose reader (Solartek Products, Inc; Alameda, Calif.). The mice were fasted for 12 hours before glucose measurement. PBS, rAAV-GFP, or rAAV-mKL were carefully injected into 10-weeks old lean and db/db mice at the dose of 2.57×10⁹ of viral genome copies/g (bw) in a total volume of 500 μL at the region of pancreas starting from the splenic lobes of pancreas towards the duodenal lobes of pancreas via IP delivery.

Glucose tolerance test and insulin sensitivity test. Glucose tolerance test (GTT) was performed during weeks 2, 4, and 6 after the treatments. Insulin sensitivity test (IST) was performed during weeks 3 and 5 after the treatments. Briefly, blood glucose levels were measured at 30, 60, 90 and 120 minutes after subcutaneous injections of D-glucose (1 g/Kg, Fisher Scientific) or insulin (1.0 U/Kg, Sigma). The baseline glucose levels were determined before the injections of glucose or insulin after 12-hour fasting.

Tissue collections. At the end of week 6 of the treatments, 5 animals from each treatment group were euthanized with an overdose of sodium pentobarbital (100 mg/kg, i.p.) and blood was collected in EDTA. The plasma samples were stored at −80° C. Following blood collections, animals were perfused transcardiacally using heparinized saline. One fifth of pancreas was embedded in optimal cutting temperature (OCT) compound (Tissue-Tek) on dry ice and stored at −80° C. until use. The rest of pancreas was fixed in 4% PBS-buffered paraformaldehyde (PFA) for 24 hours and then embedded in paraffin.

For preliminary experiments, BKS.Cg−+Leprdb/+Leprdb/OlaHsd mice (db/db mice) and BKS.Cg-m+/+Leprdb/OlaHsd (lean mice) (all male, 20 weeks) were purchased from Harlan (Indianapolis, USA). 4 to 5 animals from each stain were euthanized with an overdose of sodium pentobarbital (100 mg/kg, i.p.). The animals were perfused transcardiacally using heparinized saline. One forth of pancreas was used western blot analysis of Klotho protein expression. The rest of pancreas was fixed in 4% PBS-buffered paraformaldehyde (PFA) for 24 hours and then embedded in paraffin for immunohistochemical analysis of Klotho and insulin protein expression. A total of 20-25 islets were examined for each mouse.

Pancreatic islet isolation. At the end of week 6 of the treatments, 3 animals from each treatment group were euthanatized. Mouse pancreatic islets were isolated with a modified protocol as described previously (7). Briefly, collagenase-P was injected into the common bile duct of a mouse. The pancreas was then excised and digested at 37° C. The islets were first purified with premixed Histopaque gradient and then purified by handpicking the separated islets with low-retention pipette tips under a dissecting microscope. When viewed under the microscope, spherical and golden-brown particles (darker color) with diameter of 100-300 μm were considered as islets. 5 size-matched islets from each animal were cultured with RPMI1640 with 10% FBS in a 24-well plate for overnight. The rest of isolated islets were directly stored at −80° C. until use.

Glucose-stimulated insulin secretion. After overnight incubation of isolated islets in 10% FBS RPMI1640, islets were starved with KRB (125 mM NaCl, 4.74 mM KCl, 1 mM CaCl2, 1.2 mM KH2PO4, 1.2 mM MgSO4, 5 mM NaHCO3, and 25 mM HEPES, pH 7.4) supplemented with 0.1% bovine serum albumin and 2.8 mM glucose for 1 hour. Islets were washed with PBS and incubated with KRB buffer supplemented with 2.8 or 16.7 mM glucose for 1 h. Medium was collected and stored at −80° C. for measuring insulin levels.

Insulin measurement. Insulin levels in plasma or supernatants from glucose-treated islets were measured using an insulin EIA kit according to the manufacturer's instruction (ALPCO Diagnostics, Salem, N.H.).

Immunohistochemistry (IHC). A series of 5-μm-thick sections of paraffin-embedded pancreas were cut at a 100-μm interval on three levels. For immunohistochemical analysis, consecutive pancreatic cross sections were deparaffinized, rinsed in xylene, and rehydrated. After heat-induced antigen retrieval in a microwave, the cross sections were blocked first with 3% hydrogen peroxide. The sections were incubated with antibodies against GFP (ab290, Abcam), Flag-tag (ET-DY100, Aves Labs), Klotho (R&D SYSTEM), insulin (sc-9168, Santa Cruz Biotechnology), Pdx-1 (AB3503, Millipore), 4-hydroxyonenal (4-HNE) (ab48506, Abcam), DNAJC3 (ab70840, Abcam), PCNA (ab2426, Abcam), or LC3 (L1564-50A, US Biological) overnight at 4° C. and then with appropriated secondary antibodies conjugated with HRP at room temperature for 60 minutes. Stable diaminobenzidine (DAB, Invitrogen) was used as a substrate for peroxidase. Hematoxylin was used as counterstaining. The islets of Langerhans in the cross-sections of pancreas for each mouse were located under a microscopy (Nikon Eclipse Ti). Images of islets from consecutive cross sections for each animal were collected at equal exposure conditions and at the same magnification (40× objective lens). The staining for Klotho, insulin, Pdx-1, or LC3 staining was quantified using Image J (NIH freeware) as mean gray value/pixel. Briefly, the selection line was drawn along the islet of Langerhans after the original RGB image was converted to a gray scale image. A TUNEL assay on the cross-sections of mouse pancreas was performed using TACS•XL®-Blue Label in Situ Apoptosis Detection Kit (Trevgen, Gaithersburg, Md.). The number of cells with positive insulin, 4-HNE, DNAJC3, TUNEL, or PCNA-staining in the islet was counted in NIS-Elements BR 3.0 (Nikon). A total of 20-25 islets were examined for each animal.

In situ measurement of superoxide. Dihydroethidium (DHE) (D7008, Sigma) was used to measure superoxide levels in pancreatic islets as described previously (6). Briefly, 6-μm-thick cross sections of frozen OCT compound-embedded pancreas were cut on a cryomicrotome and fixed with 4% paraformaldehyde for 10 minutes. 2.5 μM DHE was added to the sections and incubated at 37° C. for 20 minutes. Fluorescence images of ethidium-stained islets for each sample were collected at equal exposure conditions under Nikon Eclipse Ti microscopy (magnification ×400) with the software NIS-Elements BR 3.0 (Nikon). Mean fluorescence density of pancreatic islets (25-20 islets/mouse) was analyzed using NIS-Elements BR 3.0.

RNA isolation and real time RT-PCR. Total RNA was purified from isolated mouse pancreatic islets using TRizol® Reagent, followed by Qiagen RNeasy® Mini Kit. RNA (2 μg) was reverse-transcribed using SuperScript™ III Reverse Transcriptase with random hexamer in the presence of 10 ul dNTP for 1 hour at 50° C. The resulting cDNAs were used as templates for real time PCR with oligonucleotides primers to amplify the mRNAs of insulin I (F: 5′-CCTGTTGGTGCACTTCCTAC-3′ (SEQ ID NO:14); R: 5′-TGCAGTAGTTCTCCAGCTGG-3′ (SEQ ID NO:15); size: 317 bp), insulin II (F: 5′-AGCCCTAAGTGATCCGCTACAA-3′ (SEQ ID NO:16); R: 5′-CATGTTGAAACAATAACCTGGAAGA-3′ (SEQ ID NO:29); size: 178 bp), Pdx-1 (F: 5′-CCACCCCAGTTTACAAGCTC-3′ (SEQ ID NO:17); R: 5-′ACGGGTCCTCTTGTTTTCCT-3′ (SEQ ID NO:18); size: 315 bp), DNAJC3, (F; 5′-AAGCCCGTGGAAGCCATTAG-3′ (SEQ ID NO:19); R: 5′-GGTCATTTTCATTGTGCTCCTGAG-3′ (SEQ ID NO: 20); size: 160 bp), PCNA (F; 5′-TAAAGAAGAGGAGGCGGTAA-3′ (SEQ ID NO:21); R: 5′-TAAGTGTCCCATGTCAGCAA-3′ (SEQ ID NO:22); size: 175 bp), and LC3 (F: 5′-CGAGCGCTACAAGGGTGAG-3′ (SEQ ID NO:23); R: 5′-CCGGATGATCTTGACCAAC-3′ (SEQ ID NO:24); size: 100 bp) and β-actin (F: 5′-AGGTCATCACTATTGGCAACGA-3′ (SEQ ID NO:25); R: 5′-CACTTCATGATGGAATTGAATGTAGTT-3′ (SEQ ID NO:26); size: 118 bp. Real-time PCR was performed on a Bio-Rad CFX96™ Real-Time PCR Detection Systems. PCR were cycled 40 times using the following conditions: 95° C. for 5 s, 58° C. for 5 s. Homogeneity of PCR products from each reaction was confirmed by melt curve analysis and 1.5% agarose gel analysis.

Statistical Analysis. Blood glucose and body weight were analyzed by a repeated measures one-way ANOVA. The remaining data were analyzed by one-way ANOVA. The Newman-Keuls procedure was used to reveal differences between groups. A probability value with p<0.05 was considered to be statistically significant.

Results of Example 1:

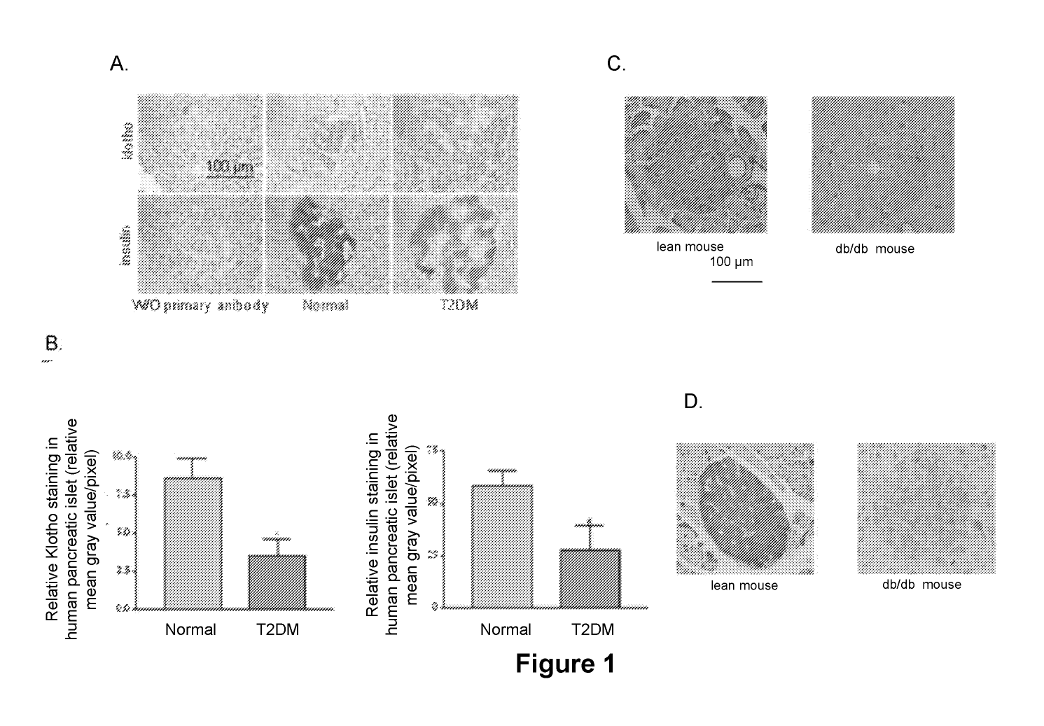

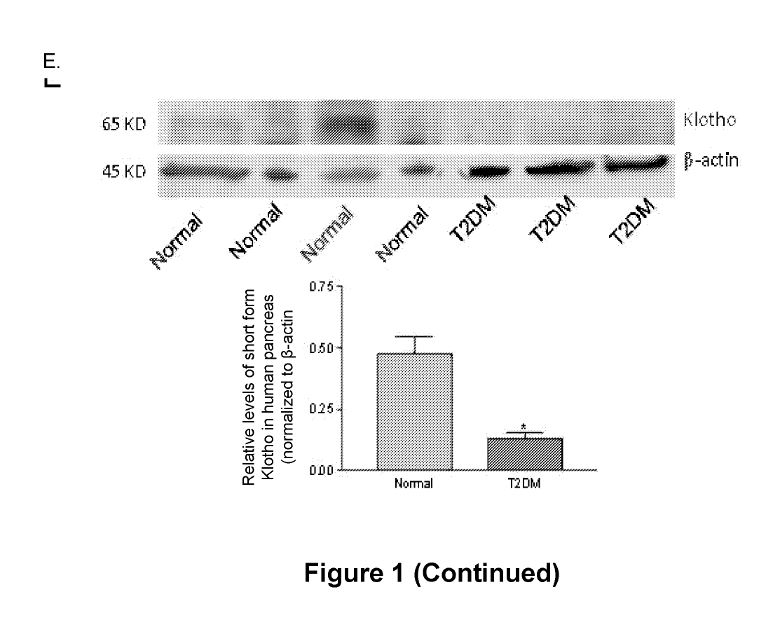

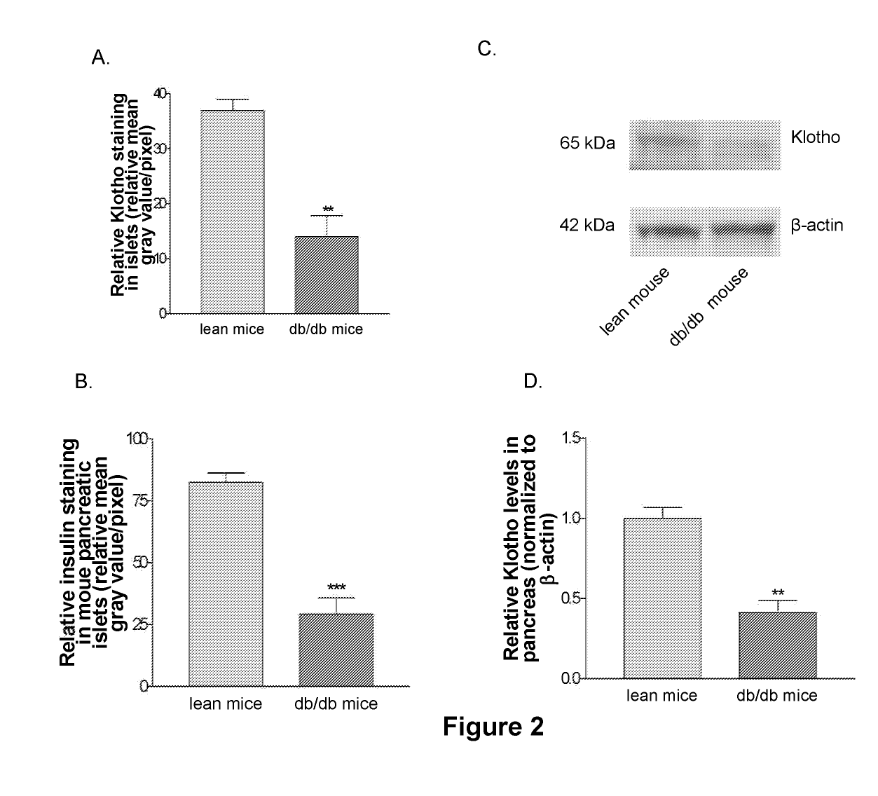

T2DM patients and diabetic mice displayed lower levels of Klotho in pancreatic islets. First, immunohistochemical staining of Klotho and insulin in human pancreas was performed. Both Klotho and insulin staining significantly decreased in pancreatic islets of patients with T2DM compared to samples from normal donors (FIG. 1a, 1b). Second, immunohistochemical analysis also showed a significant decrease in Klotho protein levels in pancreatic islets in db/db mice (FIG. 1c, 1d, 2a, 2b). The insulin storage was also decreased significantly in pancreatic islets in db/db mice compared to lean mice (FIG. 1c, 1d, 2a, 2b). Klotho is expressed in mouse pancreatic islets of Langerhans with an apparent molecular weight of 65 kDa. Third, the Klotho protein levels in human pancreas were investigated via Western blot. Klotho protein was significantly decreased in pancreas from patients with T2DM (FIG. 1e). In addition, western blot analysis indicated that Klotho protein expression levels were decreased significantly in pancreas in db/db mice (20 weeks old) compared to the age-matched lean mice as indicated by (FIG. 2c, 2d). These results indicate that Klotho expression is down-regulated in pancreatic β cells both in patients with T2DM and in db/db mice.

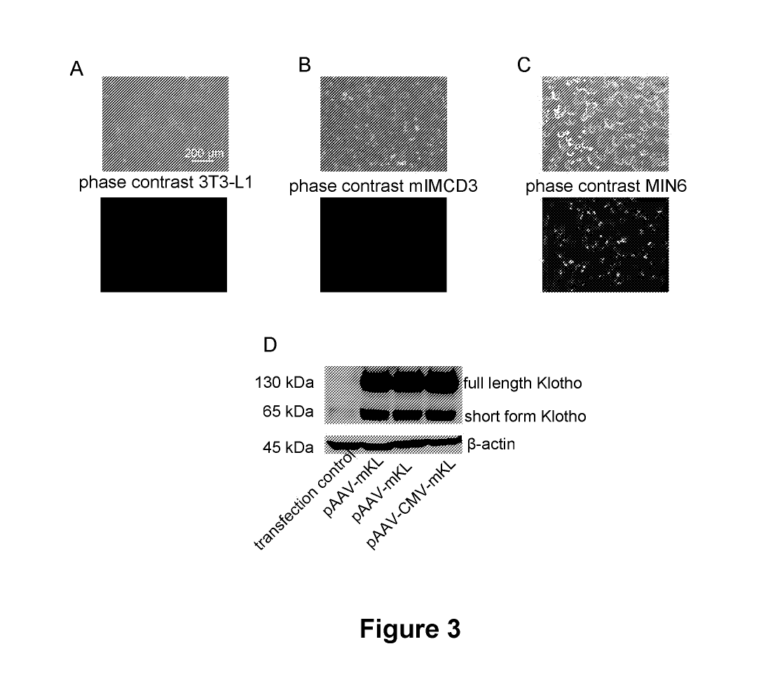

β cell-specific expression of mKL in vitro. To test the β cell specificity of the mouse insulin II promoter, MIN6 β cells, 3T3-L1 preadipocytes, and mlMCD3 cells were transfected with 0.072 μg/mL of pAAV-GFP, pAAV-mKL, or pAAV-CMV-mKL DNAs for 48 hours. In pAAV-GFP and pAAV-mKL, the original CMV promoter was replaced by the mouse insulin II promoter. Interestingly, pAAV-GFP (GFP protein) was specifically expressed in MIN6 cells but not in 3T3-L1 or mlMCD3 cells (FIG. 3a-c), indicating that the insulin II promoter is 1 cell-specific. The constructed insulin II promoter and the conventional CMV promoter are equally potent in driving mKL expression in MIN6 β cells (FIG. 3d).

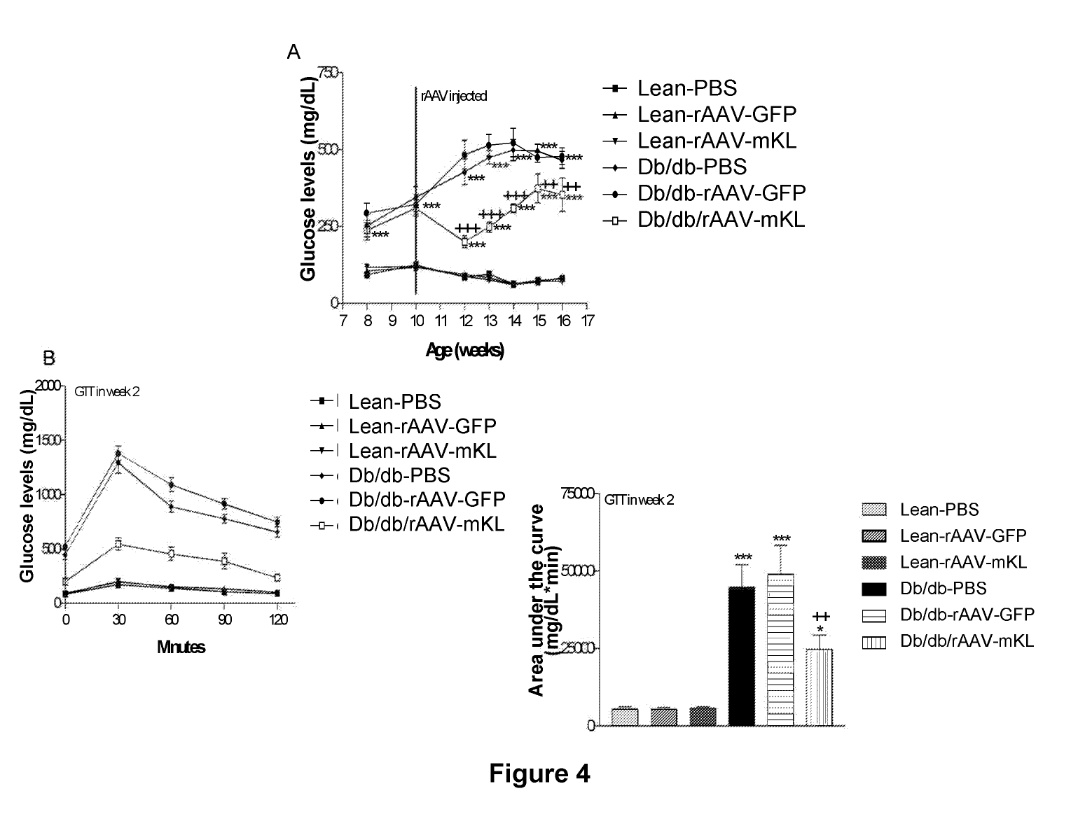

β cell-specific expression of mKL attenuated the development of diabetes in db/db mice. rAAV-GFP or rAAV-mKL were carefully injected into the region of pancreas via IP delivery in lean and db/db mice. These db/db mice were developing severe hyperglycemia at the age of 10 weeks (FIG. 4a). Interestingly, rAAV-mKL significantly attenuated hyperglycemia and dampened the development of overt diabetes in db/db within 2 weeks, compared to the PBS and rAAV-GFP treated control groups (FIG. 4a). The anti-hyperglycemic effects of rAAV-mKL were sustained for 6 weeks (length of the study) although it did not eventually prevent the rise of blood glucose (FIG. 4a). The rAAV-mKL did not alter blood glucose levels significantly in lean mice (FIG. 4a).

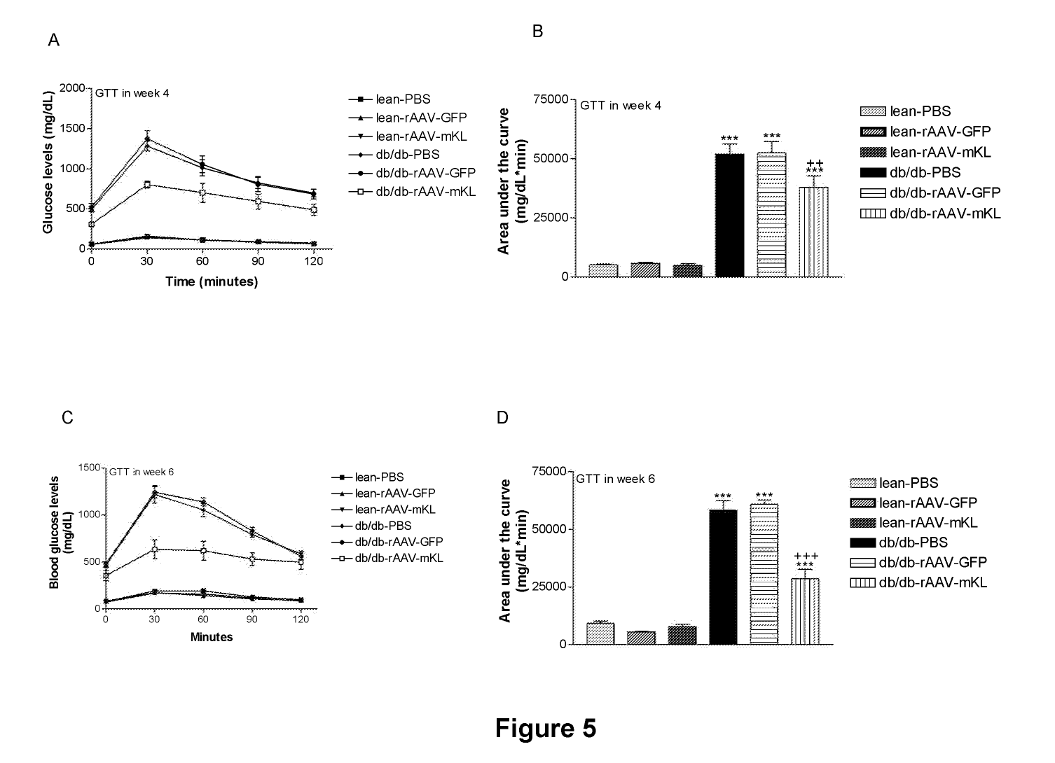

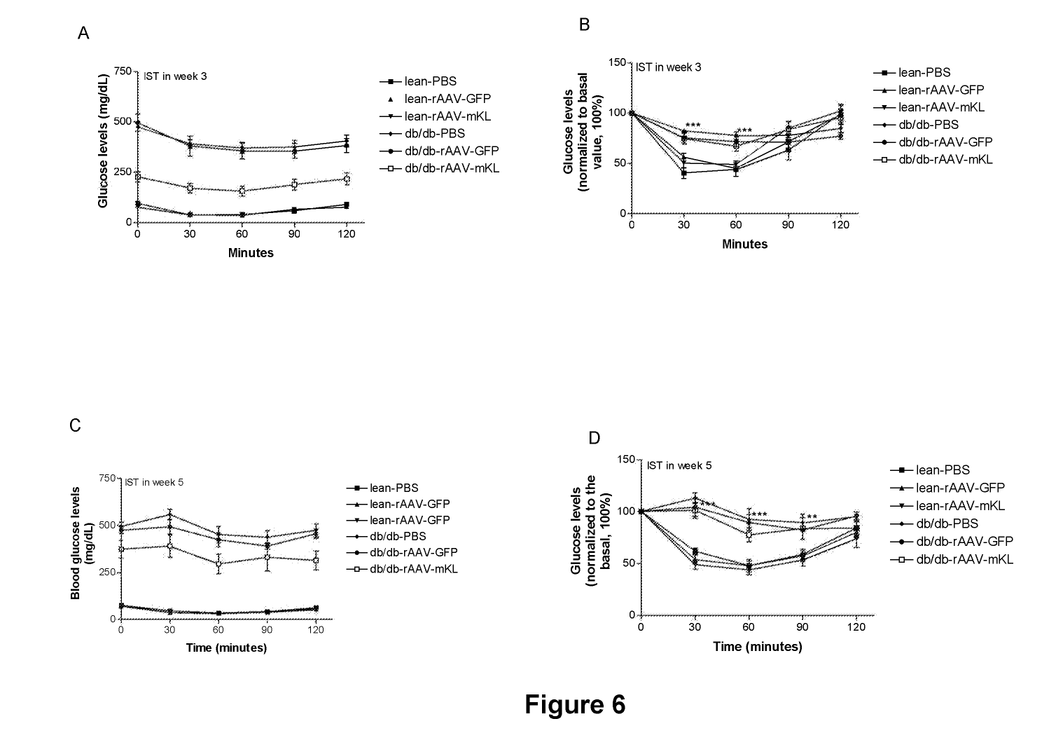

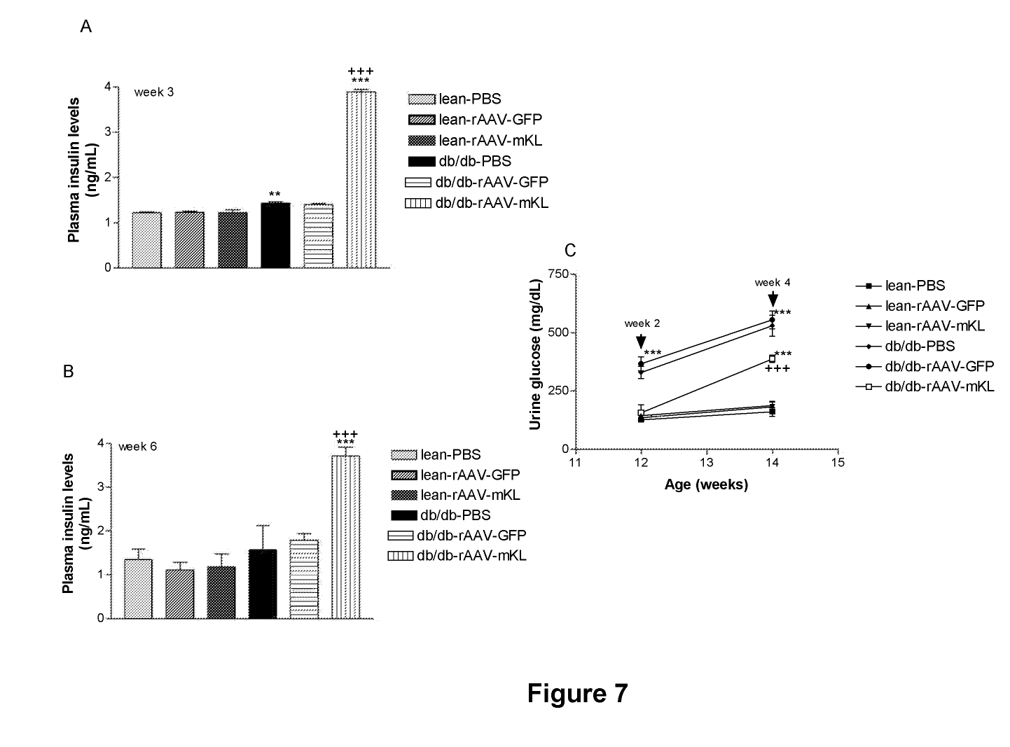

To gain insights into the mechanism of the Klotho action, the glucose tolerance test (GTT) was performed at weeks 2, 4, and 6 and the insulin sensitivity test (IST) at weeks 3 and 5 following klotho gene delivery. The db/db mice displayed overt glucose intolerance vs the lean mice (FIG. 4b-c, FIG. 5a-d). Treatments with rAAV-mKL markedly improved glucose tolerance in db/db mice (FIG. 4b-c, FIG. 5a-d). Glucose tolerance was not altered by rAAV-mKL in lean mice (FIG. 4b-c, FIG. 5a-d). The db/db mice developed severe insulin resistance (FIG. 6a-d). β cell-specific expression of klotho did not affect insulin sensitivity in either db/db or lean mice (FIG. 6a-d). These data indicate that the treatments with rAAV-mKL may directly target pancreatic β cells rather than peripheral tissues in diabetic mice. The db/db-PBS group displayed slightly higher levels of plasma insulin compared to the lean-PBS group (FIG. 7a-b). rAAV-mKL significantly further increased plasma insulin levels in db/db mice but not in lean mice (FIG. 7a-b). These results suggest that rAAV-mKLs may increase insulin release in db/db mice. Fasting urine glucose levels in db/db mice were significantly higher compared to those of lean mice, and rAAV-mKL significantly decreased urine glucose levels in db/db mice at weeks 2 and 4 after gene delivery (FIG. 7c).

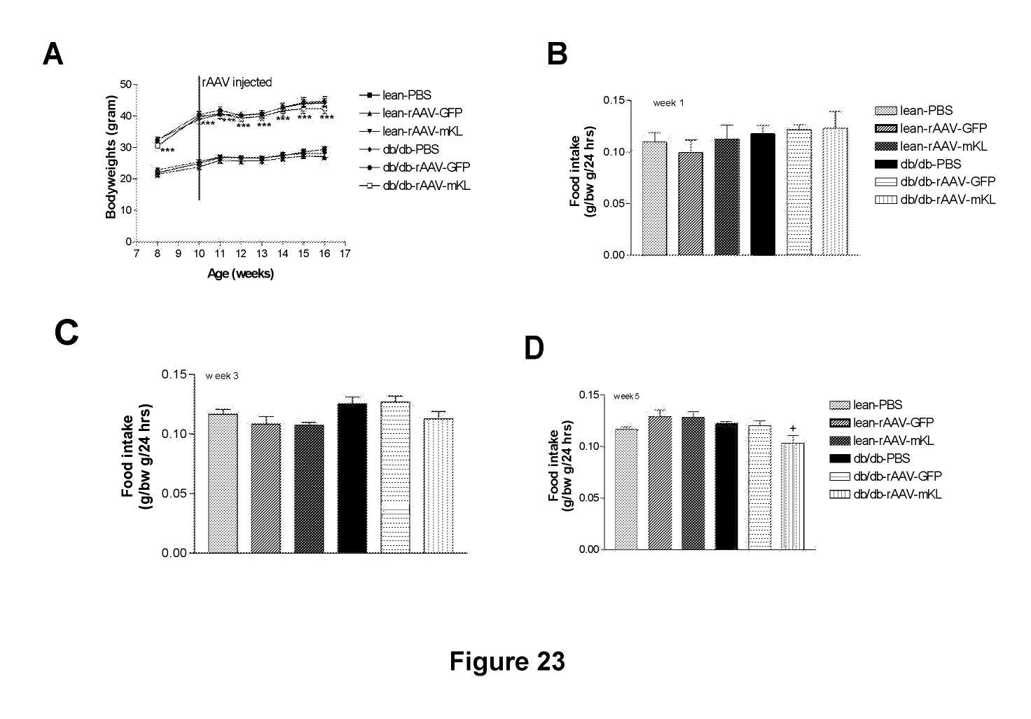

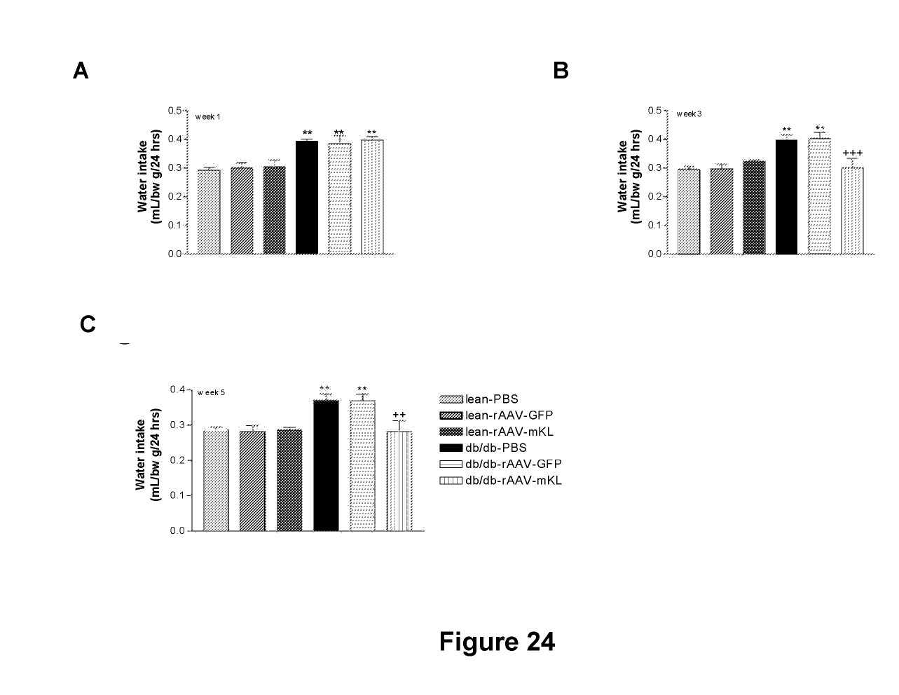

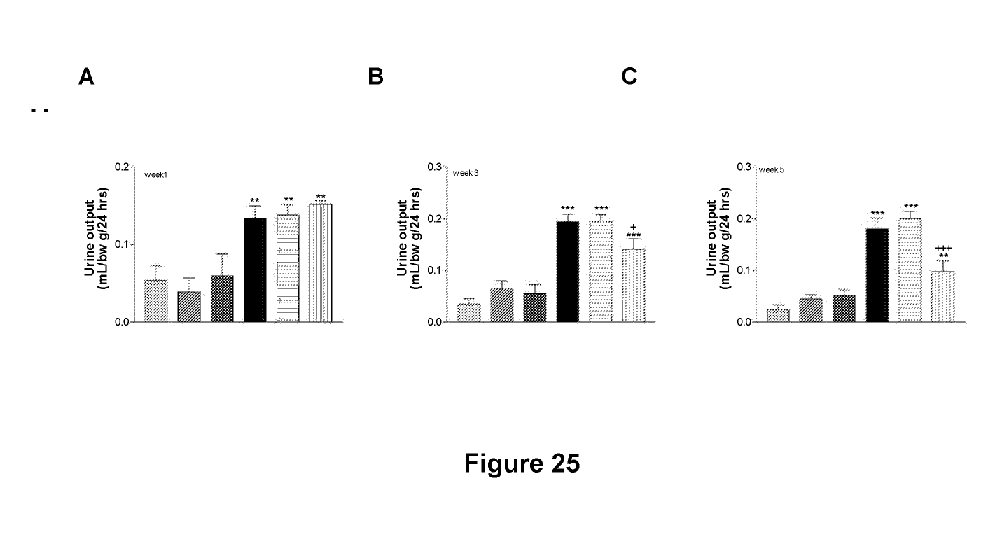

Effects of β cell-specific expression of mKL on body weight, food intake, water intake, and urine output in diabetic mice. The control db/db mice displayed much greater body weights compared to the control lean mice (FIG. 23a). β cell-specific delivery of mKL did not significantly affect the body weights in either lean or db/db mice (FIG. 23a). The rAAV-mKL slightly but significantly decreased food intake in db/db mice (normalized to body weight) at week 5 after gene delivery (FIG. 23b-d). The control db/db-PBS group had greater water intake and urine output vs the lean mice (FIG. 24a-c, FIG. 25a-c). rAAV-mKL attenuated both water intake and urine output in diabetic mice at week 3 and 5 after gene delivery (FIG. 24a-c, FIG. 25a-c).

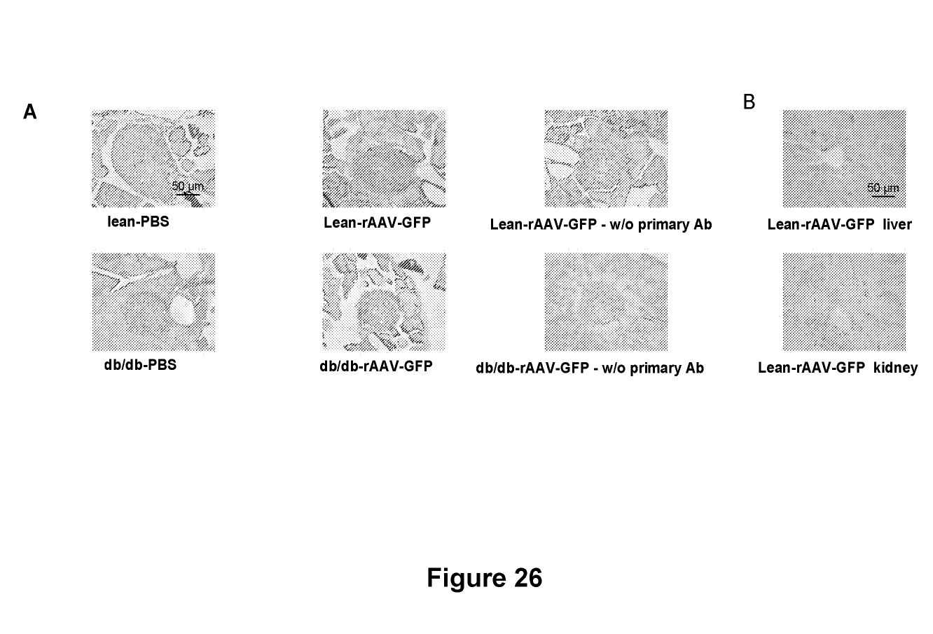

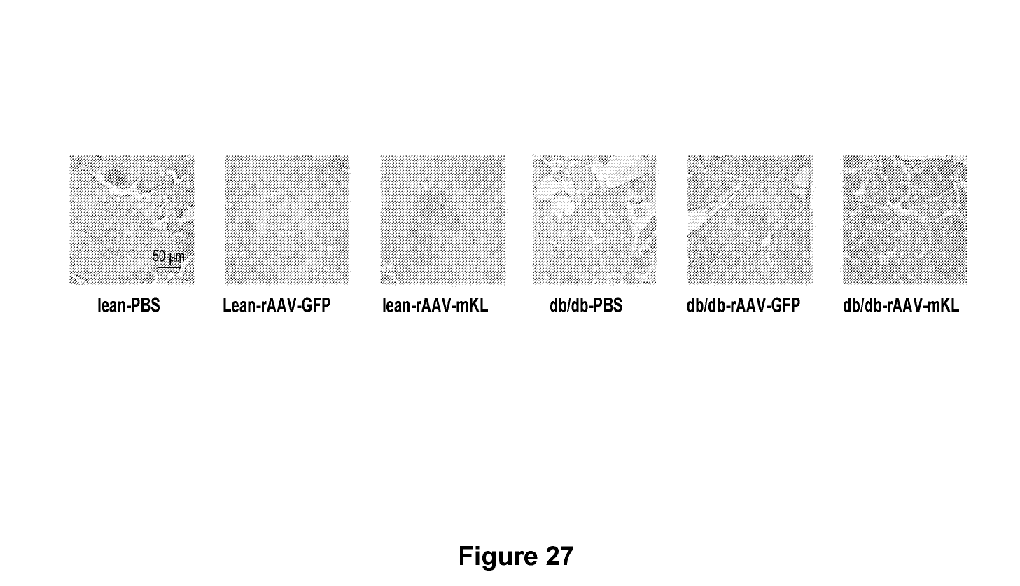

Immunohistochemical and functional analysis of mouse pancreatic islets of Langerhans. Different serotypes of rAAV with single or double-stranded DNA have been used in pancreatic islet gene transfer with various efficiencies via different routes (1, 8, 9). Given that the insert genes ranged from 0.7 to 3.1 kb, AAV2 vector with single stranded DNA was used in the gene transfer. A Flag-tog sequence had been inserted at the 3′ end of mouse Klotho gene. At 6 weeks after gene delivery, GFP and FLAG-tag protein expression were first examined in cross-sections of paraffin-embedded pancreatic islets, livers, or kidneys via immunohistochemistry. Obviously, rAAV-GFP drove GFP expression in pancreatic islets of lean and db/db mice whereas GFP was not detectable in livers and kidneys of animals injected with rAAV-GFP (FIG. 26a-b). In addition, rAAV-mKL drove FLAG-tag expression in islets of animals treated with rAAV.-mKL (FIG. 27). Thus, the intraperitoneal delivery of rAAV coupled with mouse insulin II promoter led to islet-specific gene transfer in mice.

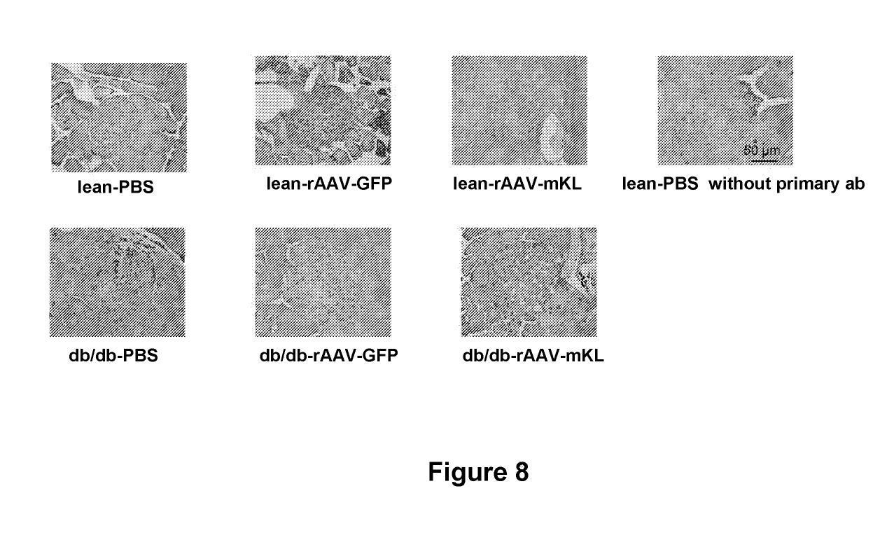

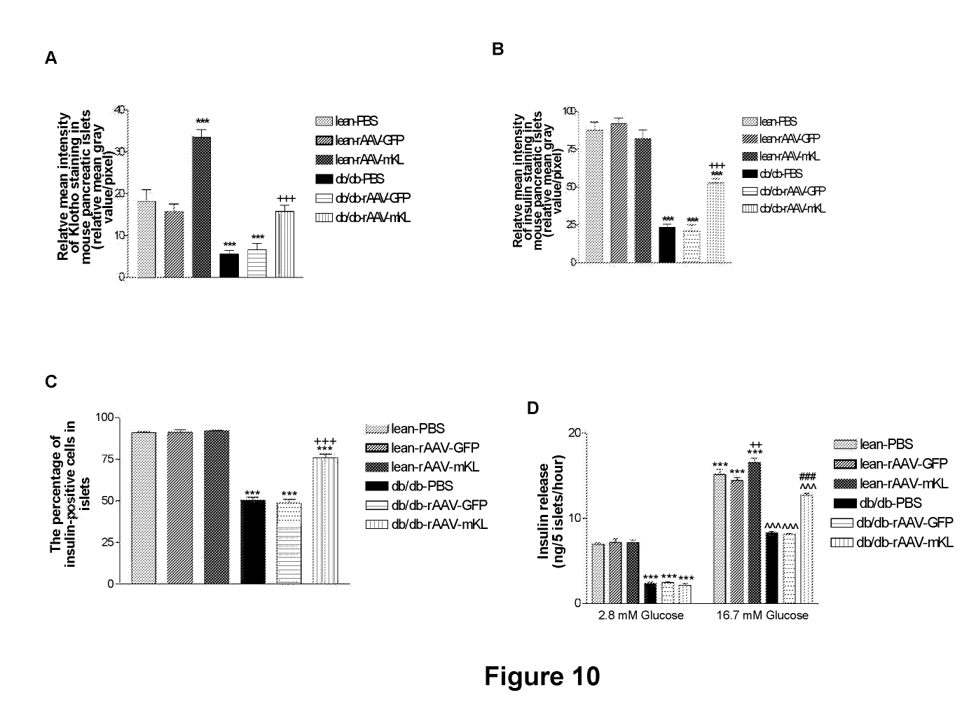

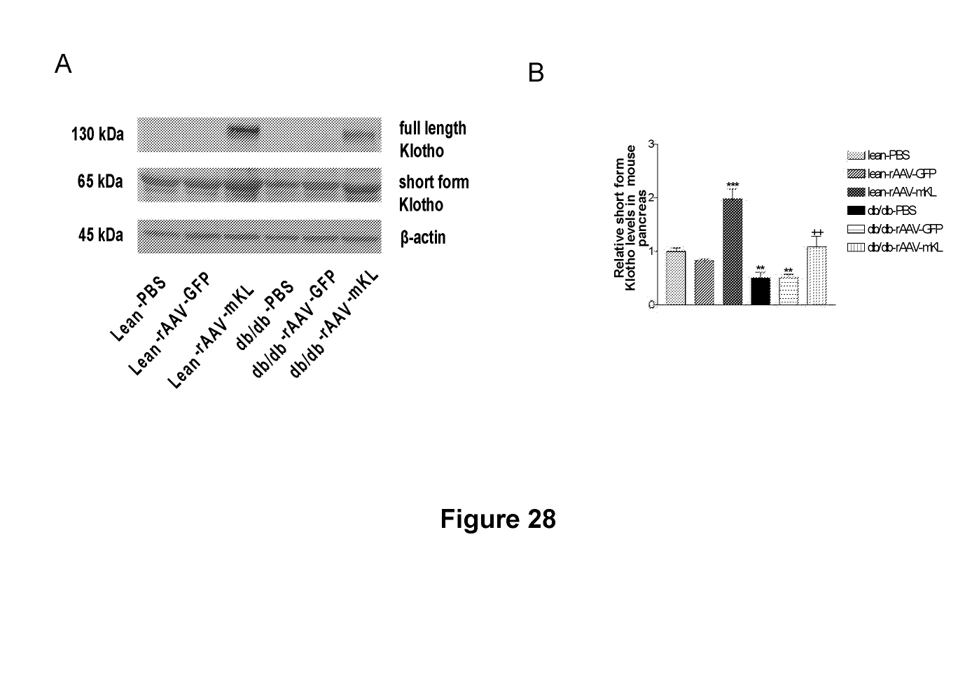

Secondly, Klotho expression in pancreatic islets in diabetic mice was studied. Klotho staining in pancreatic islets of Langerhans in control db/db mice was significantly decreased compared to that of control lean mice (FIG. 8, FIG. 10a). The treatments with rAAV-mKL increased Klotho staining in pancreatic islets of both lean and db/db mice (FIG. 8, FIG. 10a). Western blot analysis also showed that the treatments with rAAV-mKL increased Klotho protein expression in pancreas of lean and db/db mice (FIG. 28a-b).

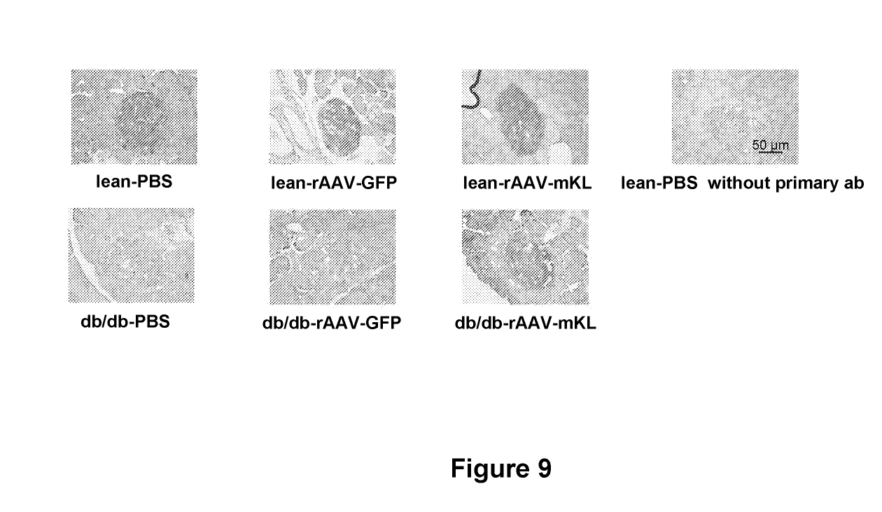

To investigate whether the β cell-specific expression of mKL exerts beneficial effects on pancreatic islets of Langerhans, insulin staining in pancreatic cross sections were performed. Insulin staining in pancreatic islets in db/db mice was significantly decreased compared to that of lean mice (FIG. 9, FIG. 10b). Interestingly, β cell-specific expression of mKL increased insulin staining in pancreatic islet of db/db mice by 1.26 folds (FIG. 10b). In addition, the number of insulin-positive cells in pancreatic islets of Langerhans in db/db mice was significantly less than that of lean mice (FIG. 10c). In contrast, β cell-specific expression of mKL partially restored the number of insulin-positive cells in pancreatic islets in db/db mice (by 55%) (FIG. 10c).

Glucose-stimulated insulin secretion was further tested in isolated mouse pancreatic islets (ex vivo). Insulin secretion was lower both at 2.8 mM and 16.7 mM glucose in islets isolated from db/db mice compared to that of lean mice (FIG. 10d). β cell-specific expression of mKL promoted insulin secretion in response to 16.7 mM glucose but not 2.8 mM glucose in islets of lean and db/db mice (FIG. 10d). These results revealed, for the first time, that the pancreatic islets of db/db mice are losing the compensatory ability in response to increased blood glucose levels and that β cell-specific expression of mKL improved the functional response of pancreatic β cells to the glucose challenge in db/db mice.

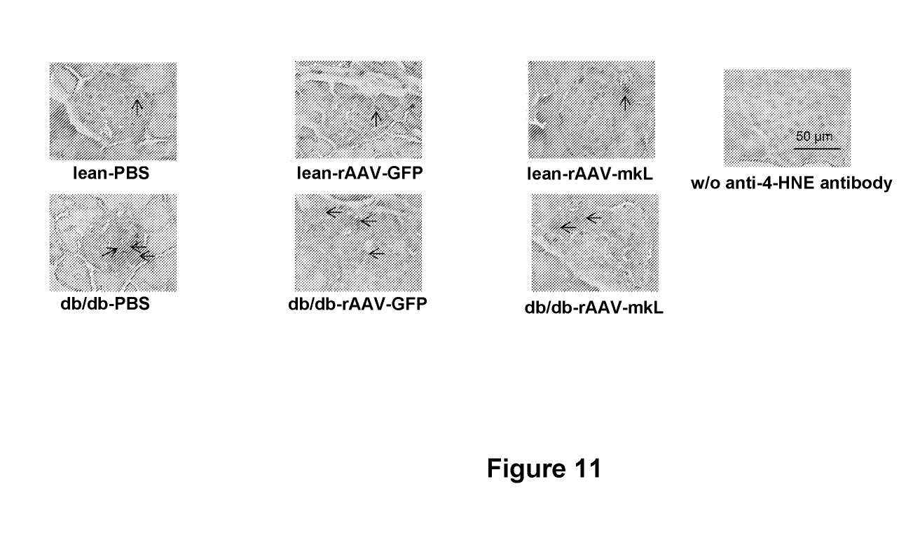

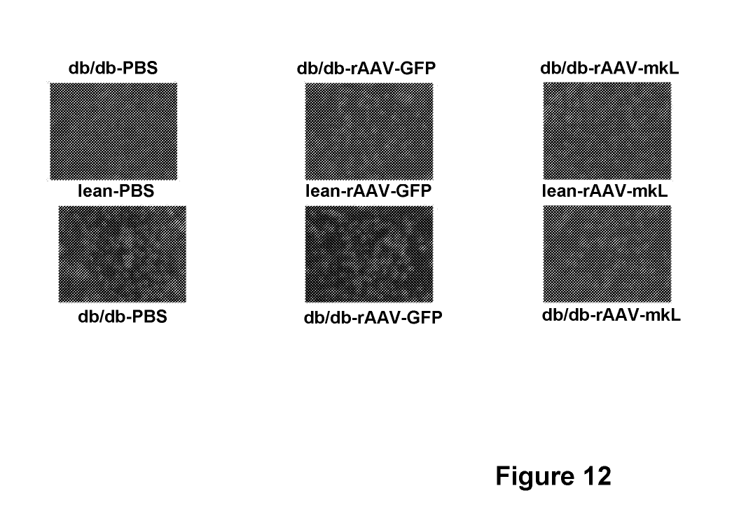

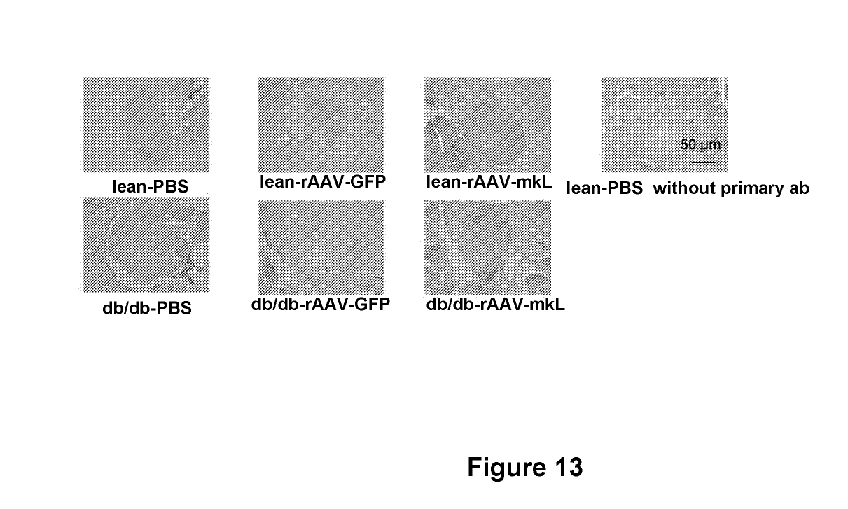

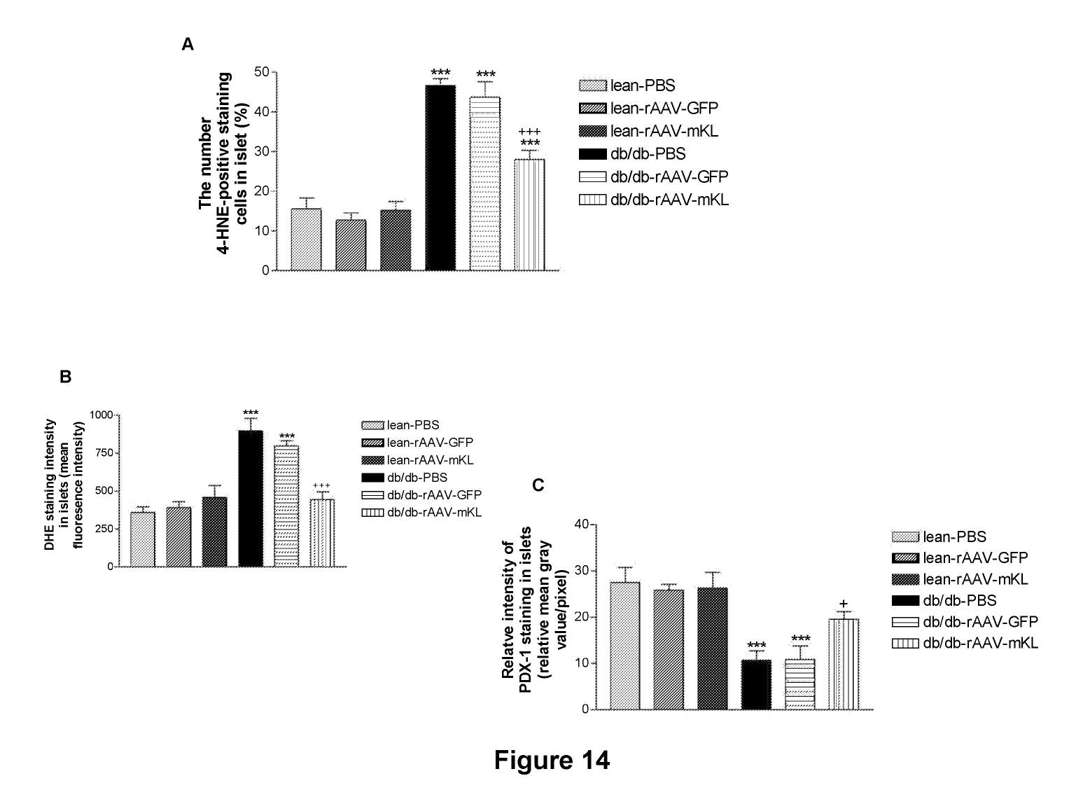

Effects of β cell-specific expression of mKL on oxidative stress, superoxide levels, and Pdx-1 expression in pancreatic islets of db/db mice. To study the mechanisms for the preservation of β cell function by Klotho in db/db mice, oxidative stress markers (4-HNE), intracellular superoxide (DHE staining), and insulin transcription factors (Pdx-1) were evaluated in pancreatic islets. The number of 4-HNE-positive cells and the intracellular superoxide level were significantly increased in pancreatic islets of db/db mice (FIG. 11, FIG. 12, FIG. 14a-b), indicating oxidative damage. In vivo expression of mouse Klotho attenuated the oxidative stress levels in pancreatic islets of db/db mice (FIG. 11, FIG. 12, FIG. 14a-b). In addition, the Pdx-1 expression level (staining) was significantly lower in pancreatic islets of db/db animals while β cell-specific expression of mKL increased Pdx-1 in islets of db/db mice (FIG. 13, FIG. 14c). Thus, the beneficial effects of Klotho on β cells may involve suppression of oxidative stress and enhancement of Pdx-1 expression in diabetic mice.

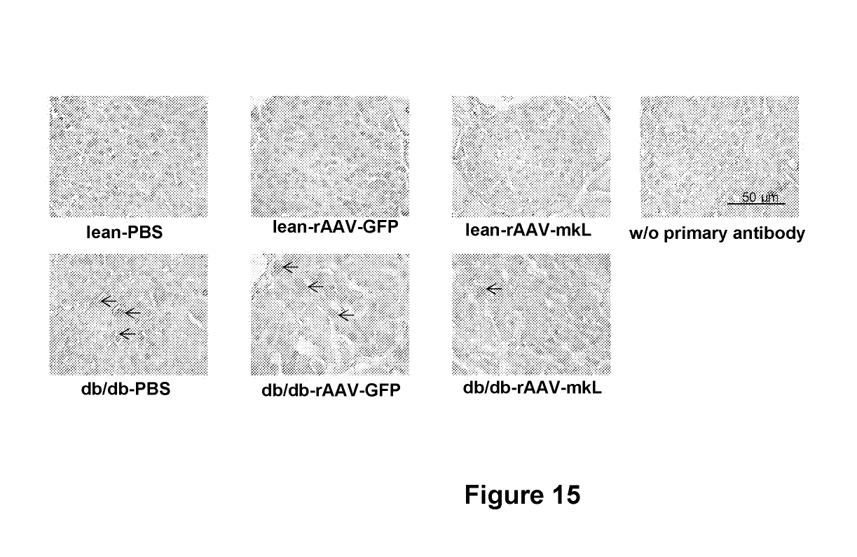

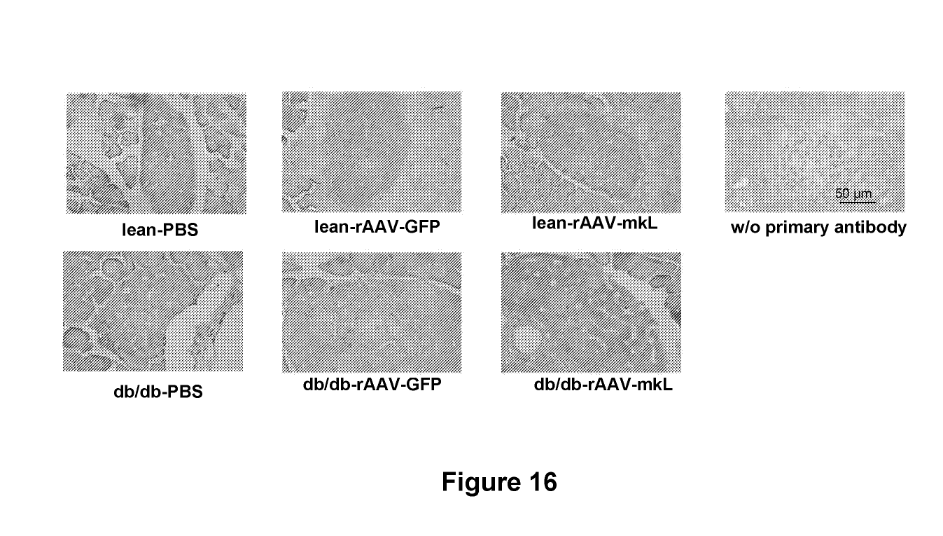

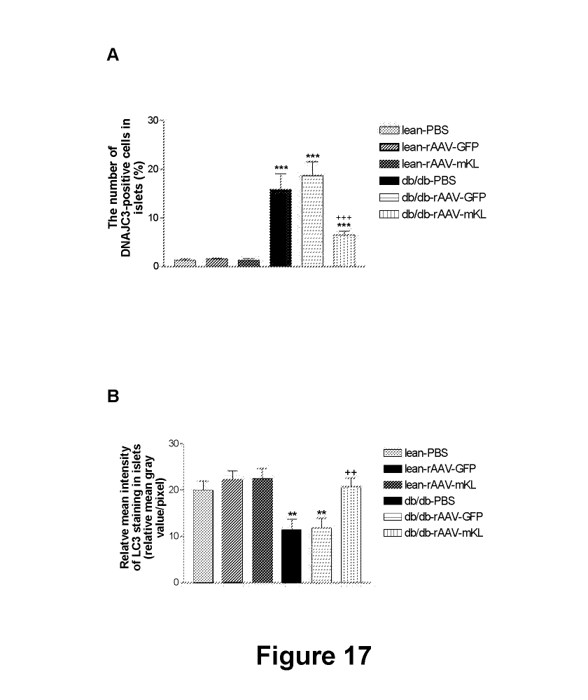

Effects of R cell-specific expression of mKL on DNAJC3, LC3, cell proliferation, and apoptosis in pancreatic islets of db/db mice. Because oxidative stress could damage cells, the ER stress marker (DNAJC3), autophagy marker (LC3), cell proliferation, and cell apoptosis were further assessed in pancreatic islets in diabetic mice. The number of DNAJC3-positive cells in the pancreatic islets was increased significantly in db/db mice vs lean mice. In contrast, β cell-specific expression of mKL decreased the number of DNAJC3-positive cells in islets of db/db mice, suggesting that expression of Klotho may suppress ER stress in islets of db/db mice (FIG. 15, FIG. 17a). LC3 staining was lower in islets of db/db mice compared to that of lean mice, suggesting that pancreatic islets of db/db mice had lower autophagic activity. Interestingly, β cell-specific expression of mKL restored autophagic activity in db/db mice (FIG. 16, FIG. 17b).

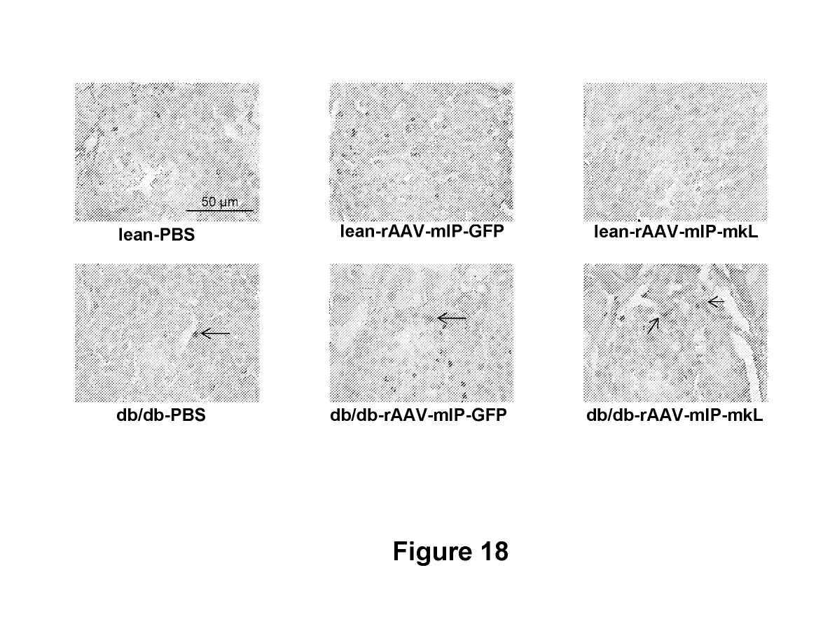

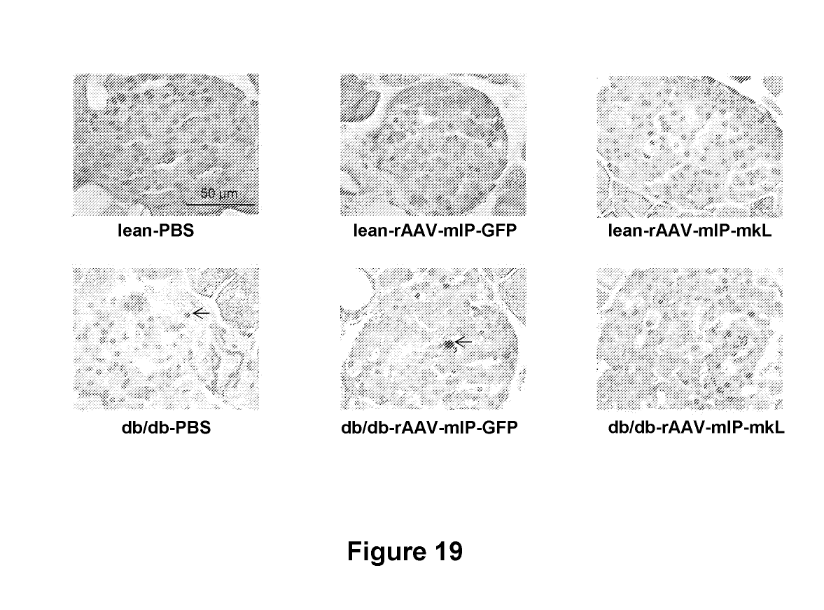

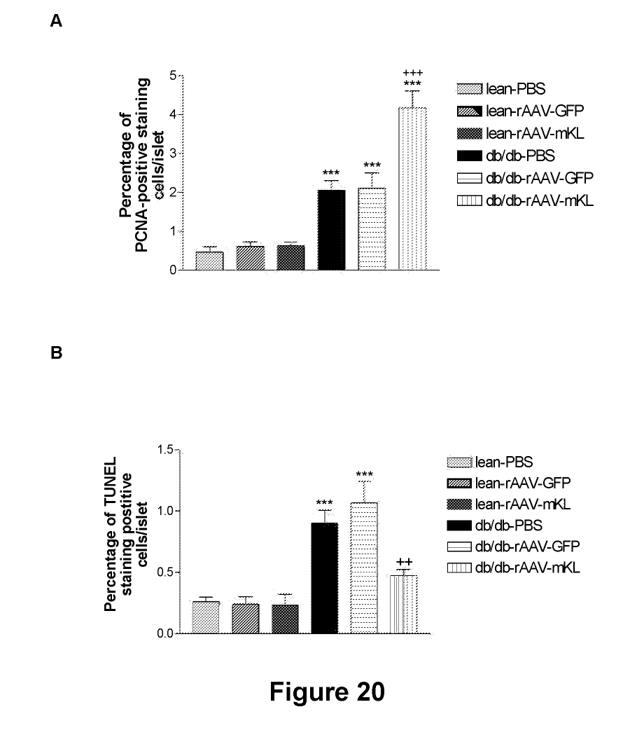

On the other hand, the number of PCNA-positive cells in islets was increased in db/db mice compared to lean mice, and β cell-specific expression of Klotho further increased the number of PCNA-positive cells in islets of db/db mice. Therefore, in vivo expression of Klotho further promoted cell proliferation in pancreatic islets of db/db mice (FIG. 18, FIG. 20a). The number of apoptotic cells was increased in islets of db/db mice, which can be dampened by expression of Klotho. Thus, rAAV-mKL treatments attenuate apoptosis in pancreatic islets of db/db mice (FIG. 19, FIG. 20b).

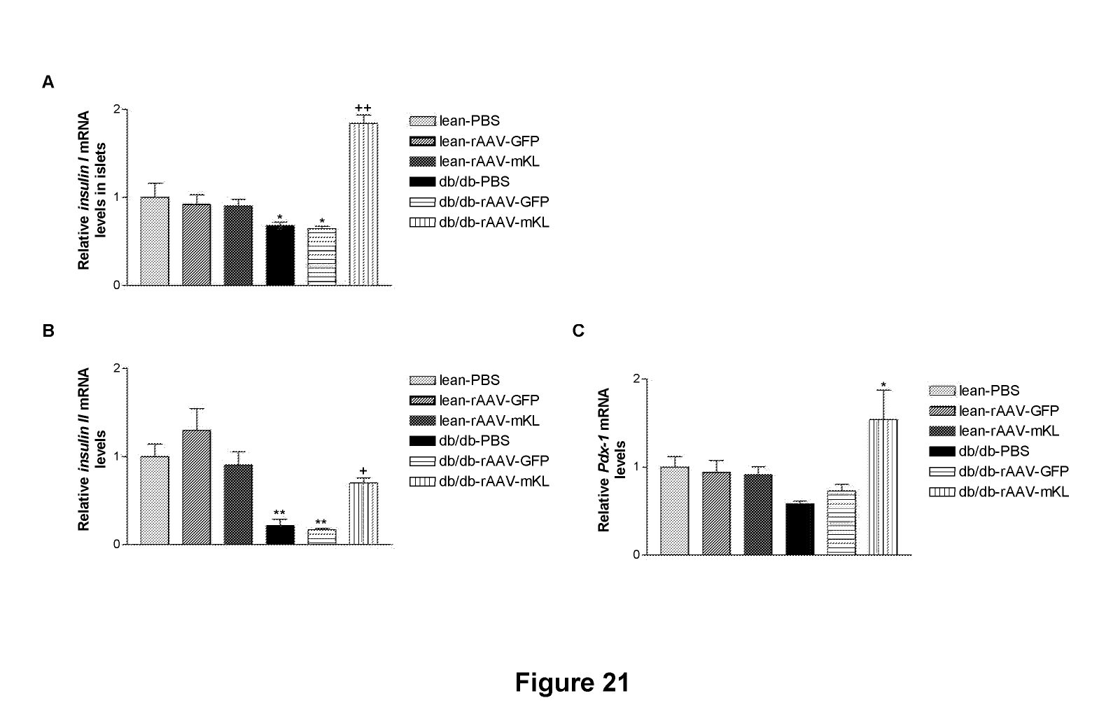

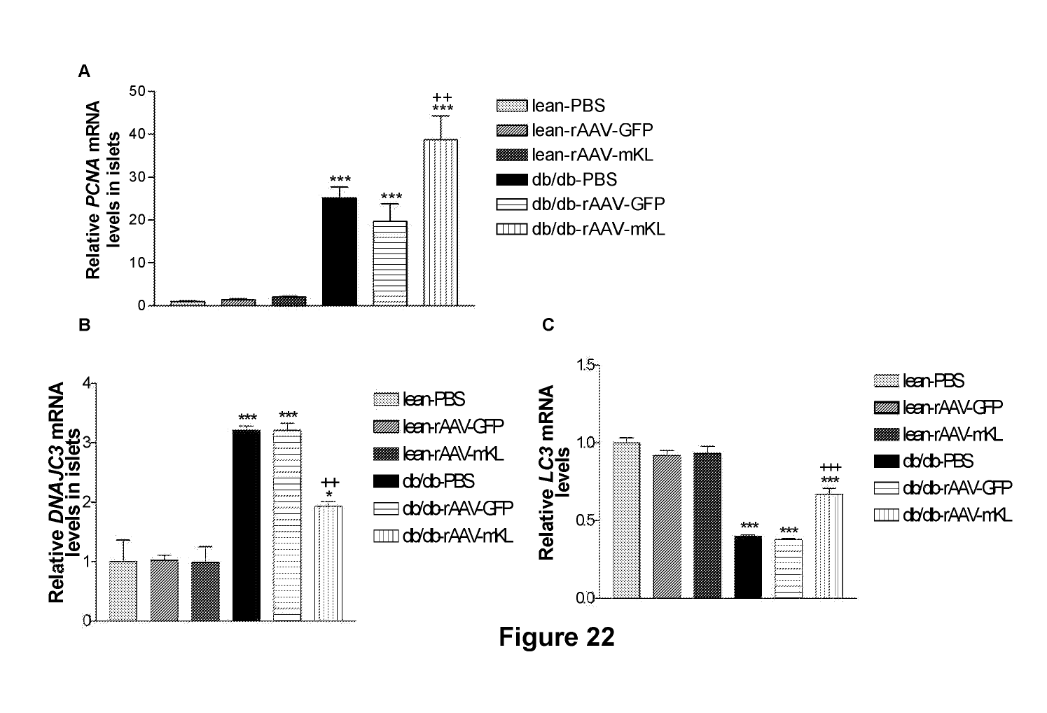

Effects of the β cell-specific expression of mKL on gene expressions in islets of diabetic mice. It was also assessed if Klotho affects the corresponding gene expressions in line with the changes in several proteins involved in the preservation of pancreatic islets. The db/db mice displayed significantly lower mRNA expression levels of insulin I, Insulin II, Pdx-1, and LC3, while the treatments with rAAV-mKL increased mRNA levels of these genes in islets in diabetic mice (FIG. 21a-c, FIG. 22c). In addition, db/db mice had higher mRNA expression levels of DNAJC3 and PCNA while the rAAV-mKL treatments decreased DNAJC3 mRNA expression but further increased PCNA mRNA expression levels in islets (FIG. 22a-b). These data suggest that the β cell-specific expression of mKL preserved β cells via regulating gene expressions of insulin I, insulin II, Pdx-1, LC3, PCNA, and LC3 mRNA.

Discussion of Example 1:

The pancreatic β cells are essential to the regulation of glucose homeostasis. Substantial β cell failure is now believed to occur at an early stage in the progression of T2DM. Thus, one of the goals in the treatment of T2DM is to preserve functional β cells. Klotho, a recently-discovered aging-suppressing gene, was believed to be expressed in kidneys (10). As noted above, the Klotho gene and protein have been found to be expressed in pancreatic islets. Notably, Klotho protein expression in β cells was decreased in db/db mice, a mouse model of T2DM (FIG. 1c, 2a). A finding of the present work is that β cell-specific expression of Klotho administered by gene therapy attenuates the development of diabetes and enhances the performance in glucose tolerance in db/db (diabetic) mice. Without wishing to be bound by theory, it is believed that the beneficial effects of Klotho added by gene therapy are due to the preservation and increase in the number of insulin-positive β cells, insulin storage levels in pancreatic islets, and the glucose-stimulated insulin secretion from pancreatic islets which led to the increased blood insulin levels in db/db mice.

It is noticed that the depleted klotho protein expression was associated with the decreased insulin storage in pancreatic islets (FIG. 2b) and the impaired glucose-stimulated insulin release in pancreatic islets of db/db mice (FIG. 10d). β cell-specific expression of Klotho improved or preserved β cell function (FIGS. 4-10). Without wishing to be bound by theory, it is new and interesting observation that β cell function appears to be regulated by Klotho. These findings are supported by cell culture studies which showed that that silencing of Klotho gene impaired glucose-stimulated insulin release and that overexpression of Klotho promoted glucose-stimulated insulin secretion in MIN6 β cell. Klotho enhances glucose-induced insulin secretion by regulating plasma membrane levels of TRPV2 and intracellular levels of calcium. Klotho-deficient mice exhibit hypoinsulinmia and pancreatic islet atrophy with diminished insulin protein and mRNA levels. The present study further demonstrated that β cell-specific expression of Klotho increased Pdx-1, insulin I, and insulin II mRNA levels and their corresponding proteins levels in pancreatic islets in db/db mice. Pdx-1 is the major regulator of glucose-stimulated insulin gene transcription. Specific point mutations in Pdx-1 have been associated with MODY (maturity-onset diabetes of the young) 4 and late-onset T2DM, characterized by a decline in β cell function. Thus, these studies suggest that the promoting effects of Klotho on insulin synthesis may be attributed partially to the increased Pdx-1 expression.

The Insulin II promoter was specific in driving gene expression in pancreatic β cells as GFP was exclusively detected in pancreatic islets and was not detectable in peripheral tissues (liver and kidneys) in mice treated with rAAV.GFP (FIG. 26a). rAAV.mKL was expressed in pancreatic islets as indicated by expression of FLAG-tag (FIG. 27). The insulin II promoter was also potent in driving Klotho gene expression in β islets (FIG. 28a-b). This promoter is as potent as the CMV promoter (FIG. 28a).

The potential mechanisms by which 13 cell-specific expression of Klotho protected against β cell failure in pancreatic islets of db/db mice were further explored. rAAV-mKL decreased reactive oxygen species and oxidative damage as measured by DHE and 4-hydroxynonenal (4-HNE), respectively, in pancreatic islets of db/db mice. Oxidative stress induced by reactive oxygen species is involved in the impairment of β cell function during the development of diabetes. Because of their low antioxidant capacity, β cells are extremely sensitive to oxidative stress. Hyperglycemia and hyperlipidemia cause oxidative damage to proteins, lipids and DNA in β cells as the result of a combination of increased free radical production and an impaired ability of cells to detoxify the radicals and repair damaged molecules. By covalently modifying membrane-associated proteins, the membrane lipid peroxidation product 4-HNE may play particularly sinister roles in the metabolic syndrome and associated disease processes. 4-HNE can damage pancreatic β cells. It has been shown that expression of Klotho protein increased resistance to oxidative stress via activating the FoxO forkhead transcription factors thereby inducing expression of manganese superoxide dismutase (SOD) in Hela cells and mouse skeleton muscles. It was recently reported that Klotho gene delivery suppressed Nox2 NADPH oxidase protein expression and attenuated oxidative stress in rat aortic smooth muscle cells (5).

As a secretory cell that synthesizes and releases a large amount of insulin, the β-cell is expected to be susceptible to alterations in endoplasmic reticulum (ER) homeostasis, which can result in the accumulation of unfolded, misfolded and/or aggregated proteins (a phenomenon known as ER stress). Eukaryotic cells respond to ER stress by activating the unfolded protein response (UPR), a process that allows cells to adapt to and attempt to relieve ER stress conditions. Hyperactivation of the UPR is indispensible for ER homeostasis and may be involved in β cell dysfunction and death during the progression of T2DM. The level of ER chaperone protein DnaJ (Hsp40) homologue C3 (DNAJC3) is elevated in pancreatic islets of db/db mice and human T2DM. β cell-specific expression of mKL attenuated diabetes-induced increases in DNAJC3 expression in pancreatic islets (FIGS. 15-17). Thus, this result reveals a previously unidentified role of Klotho in the attenuation of ER stress which may contribute to the preservation of β cells and protection against diabetes by β cell-specific expression of Klotho.

The present work has demonstrated that β cell-specific expression of Klotho enhanced cell proliferation and decreased cell apoptosis in pancreatic islets of db/db mice (FIGS. 18-20). These results indicate, for the first time, that β cell-specific expression of Klotho preserves β cells partially via attenuating apoptosis and promoting cell proliferation in pancreatic islets of db/db mice. Regulation of β cell mass is dynamic and is tightly matched to meet the body's demand for insulin. The rates of β cell apoptosis or necrosis and β cell proliferation or neogenesis equilibrate at a frequency of 0.5% under steady-state conditions. It was reported that β cell apoptosis contributes to the reduction of β cell mass in patients with T2DM (11, 12). It has been shown that soluble Klotho and FGF23 together promote cell proliferation in vitro (13,14). It is noticed that the frequency of proliferative and apoptotic events per islet is relatively low (1-2%) in this diabetic model.

Unexpectedly, the expression of LC3, a marker of autophagy, was decreased in pancreatic islets of db/db mice while β cell-specific expression of mKL reversed the downregulation of LC3 expression in islets of db/db mice (FIG. 17, FIG. 17b, FIG. 22c). Autophagy is a physiologically preserved process that maintains homeostatic functions like protein degradation and organelle turnover. A major member of this family is the microtubule associated-protein 1 light chain 3 (LC3), which is associated with the autophagosome from its formation up to its maturation into autolysosome and serves as a bona fide marker for autophagy. Loss-of-function experiments (Atg7^{f/f}:RIP-Cre mice) have demonstrated that autophagy in β cells is critical in the preservation of pancreatic β cell function (15). Accumulation of p62, a substrate for autophagy, in β-cells of db/db mice has been observed. Autophagy deficiency may be involved in lipotoxicity-induced β cell failure in T2DM. Therefore, the present work indicates that the restoration of autophagic activity by expression of Klotho contributes to the preservation of β cells in pancreatic islets of diabetic mice.

In conclusion, β cell-specific expression of Klotho preserved the β cell function and protected against the development of T2DM in db/db mice. Particularly, β cell-specific expression of Klotho attenuated hyperglycemia and improved the performance in glucose tolerance in diabetic mice. This protection was associated with significant increases in the number of β cells, Pdx-1 levels, and insulin storage levels in pancreatic islets, the glucose-stimulated insulin secretion from pancreatic islets, and blood insulin levels. β cell-specific expression of Klotho preserved the β cell function likely by suppressing oxidative stress, ER stress and apoptosis, increasing cell proliferation, and normalizing autophagy in pancreatic islets of db/db mice. Therefore, in vivo expression of Klotho in pancreatic β cells offers a new and effective therapeutic strategy for β cell dysfunction in T2DM (as well as T1DM in regard to preservation of β cells) in mammals. While β cell-specific expression of Klotho attenuated the development of T2DM, simultaneous management of hyperglycemia and insulin resistance is also important for the protection of β cells in T2DM.

### Example 2

### STZ-Induced Diabetes in Heterozygous Klotho (KL^{+/-}) Mice

Multiple low doses of STZ in mice have been shown to selectively destruct 1 cells which in turn induces immune reactions against pancreatic islets leading to β cell apoptosis and subsequently T1DM. This model resembles key features of human T1DM including apoptosis and dysfunction of pancreatic β cells. In this work, protection against T1DM by Klotho via the preservation of pancreatic β cells from apoptosis induced by STZ was investigated.

Methods of Example 2:

AAV vector construction and recombinant viral production. The full length Klotho cDNA with the C-terminal Flag tog (3.1 kb in total) was cloned into AAV serotype-2 (AAV2) (Startagen, La Jolla, Calif., USA). The mouse insulin II promoter was cloned into AAV2 for replacing the original CMV promoter and intron. Insulin II promoter and GFP cDNA (700 bs) were cloned into the AAV2 vector as the control constructs. The constructs of pAAV-mKL and pAAV-GFP were then packaged with pHelper and pAAV-RC to produce recombinant AAV by following the manufactory instruction manual (Startagen, La Jolla, Calif., USA). Recombinant AAV was purified via a method of CsCl gradient (2). The titers of recombinant viral genome particles were determined using real-time PCR (Bio-Rad CFX96™ Detection Systems) with a pair of primers targeted insulin II region (F: 5′-AAATGCTCAGCCAAGGACAA-3′ (SEQ ID NO:27) and R: 5′-GGACTTTGCTGTTTGACCCATT-3′ (SEQ ID NO:28)) and with the method as described previously (3,4). Hereinafter below, these recombinant viruses will be referred as rAAV-GFP and rAAV-mKL, respectively.

Animal studies. For Klotho gene deficiency study, heterozygous Klotho (KL^{+/-}) mice with more than 9 generations in 129Sv background which were kindly provided by Dr. Kuro-o were used. The wild type (WT) littermate 129Sv mice were used as controls. For the in vivo Klotho gene delivery study, 129S1/Svlm male mice were used (Jackson Laboratory, Bar Harbor, Me. USA). All mice were housed in cages at room temperatures (25±1° C.) and were provided with Purina laboratory chow (No. 5001) and tap water ad libitum. This animal study was carried out according to the guidelines of the National Institutes of Health on the Care and Use of Laboratory Animals. This project was approved by the Institutional Animal Care and Use Committee at the University of Oklahoma Health Sciences Center. In experiments with KL^{+/-} mice, 8-10 week-old body weight-matched KL^{+/-} and wild type male mice were injected with multiple low doses of STZ (50 μg/g/day, 5 days, IP) or citrate buffer, respectively. STZ provoke β cell destruction and immune reaction against pancreatic islets. In experiments with 129S1/Svlm mice, PBS, rAAV-GFP, or rAAV-mKL were carefully injected into 8-weeks old and body weight-matched male mice at the dose of 2.57×10⁹ of viral genome copies/g (bw) in a total volume of 500 μL at the region of pancreas starting from the splenic lobes of pancreas towards the duodenal lobes of pancreas via IP delivery. One week later, these mice were injected with multiple low doses of STZ (50 μg/g/day, 5 days) or citrate buffer. Body weight was monitored weekly. Blood glucose was measured weekly from the tail vein blood using a Reli On Ultima glucose reader (Solartek Products, Inc; Alameda, Calif.). All mice were fasted for 3 hours before glucose measurement.

Glucose tolerance test and insulin sensitivity test. In experiments with KL^{+/-} mice, glucose tolerance test (GTT) was performed during weeks 2 and 4 after the initial injections of STZ. Insulin sensitivity test (IST) was performed during week 5 after the initial injections of STZ. In experiments with 129S1/Svlm mice, glucose tolerance test (GTT) was performed during weeks 3 and 5 after the initial injections of rAAV. Insulin sensitivity test (IST) was performed during week 6 after the initial injections of rAAV. Briefly, blood glucose levels were measured at 30, 60, 90 and 120 minutes after subcutaneous injections of D-glucose (1 g/Kg, Fisher Scientific) or insulin (1.0 U/Kg, Sigma). The baseline glucose levels were determined before the injections of glucose or insulin after 3-hour fasting.

Tissue collections. At the end of week 5 after the initial injection of STZ for KL^{+/-} mice and at the end of week 6 after the rAAV injection for 12951/Svlm mice, animals were euthanatized with an overdose of sodium pentobarbital (100 mg/kg, i.p.) and blood was collected. The plasma samples were stored at −80° C. Following blood collections, animals were perfused transcardiacally using heparinized saline. Pancreas was placed in 4% PBS-buffered paraformaldehyde (PFA) for 24 hours and then embedded in paraffin.

Insulin measurement. Plasma levels of Insulin were measured using an insulin EIA kit according to the manufacturer's instruction (ALPCO Diagnostics, Salem, N.H.).

Immunohistochemistry (IHC). A series of 5-μm-thick sections of paraffin-embedded pancreas were cut at a 100-μm interval. For IHC analysis, consecutive pancreatic cross sections were deparaffinized, rinsed in xylene, and rehydrated. After heat-induced antigen retrieval in a microwave, the cross sections were blocked first with 3% hydrogen peroxide. The sections were incubated with antibodies against GFP (ab290, Abcam), Flag-tag (ET-DY100, Aves Labs), Klotho (R&D SYSTEM), and insulin (sc-9168, Santa Cruz Biotechnology) overnight at 4° C. and then with appropriated secondary antibodies conjugated with HRP at room temperature for 60 minutes. Stable diaminobenzidine (DAB, Invitrogen) was used as a substrate for peroxidase. Hematoxylin was used as counterstaining. The islets of Langerhans in the cross-sections of pancreas for each mouse were located under a microscopy (Nikon Eclipse Ti). Images of islets from consecutive cross sections for each animal were collected at equal exposure conditions and at the same magnification (40× objective lens). Klotho and insulin staining was quantified using Image J (NIH freeware) as mean gray value/pixel. Briefly, the selection line was drawn along the islet of Langerhans after the original RGB image was converted to a gray scale image. The TUNEL assay on the cross-sections of mouse pancreas was performed using TACS•XL®-Blue Label In Situ Apoptosis Detection Kit (Trevigen, Gaithersburg, Md.). The number of cells with positive insulin or TUNEL staining in the islet was counted in NIS-Elements BR 3.0 (Nikon).

Cell culture. Pancreatic insulinoma MIN6 β cells were kindly provided by Dr. Miyazaki and Dr. Steiner. MIN6 β cells were cultured and maintained in DMEM containing 25 mM glucose, 10% FBS, 1% penicillin/streptomycin, 2 mM glutamine, and 100 μM β-mercaptoethanol. MIN6 cells of passage less than 25 were used in the experiments.

Transfection with plasmid DNA. Plasmid DNAs including pAAV-CMV-mKL and pAAV-CMV-GFP were constructed as described previously and purified with Qiagen Maxi Kit. For the MIN6 β cell study, pAAV-CMV-GFP and pAAV-CMV-mKL were referred as pGFP and pmKL, respectively. MIN6 β cells cultured in 6-well plate were transfected with various plasmid DNAs at the concentration of 0.072 μg/mL using Optifect™ reagent according to the manufacturer's protocol, followed by 48-hour incubation in DMEM with 10% FBS at 37° C. in a 5% CO₂ incubator.

Cell adhesion. Transfected cells (10⁵ cells/well) were seeded on 48-well tissue culture plates coated with mouse collagen IV (5 μg/mL, cat:35233, BD Biosciences) or 1% BSA and incubated in serum-free medium for 3 h. Nonadhered cells were removed, and adhered cells were thoroughly washed. Briefly, 3 to 5 random fields were imaged per well under a microscopy (Nikon Eclipse Ti). The number of cells that attached and spread were counted in NIS-Elements BR 3.0 (Nikon). For blocking experiments, transfected cells were pre-incubated with 5 μg/mL of integrin β1 blocking antibody (cat: ab24693, Abcam) or isoform control antibody IgG1 (cat: 401403, Biolegend) for one hour before they were seeded on the plate.

Apoptosis assay. Transfected cells were seeded on collagen IV-coated coverslips in a 6-well plate and incubated in DMEM containing 10% FBS for 24 hours. Cells were then in serum-free condition for overnight before incubated with STZ (1 mM), TNFα (40 ng/mL), IgG1 (5 μg/mL) or integrin β1 blocking antibody (5 μg/mL) for 24 hours. Apoptotic nuclear changes were assessed with ApopTag® Plus Peroxidase in Situ Apoptosis Detection Kit (Millipore). Briefly, 5 random fields were imaged per well under a microscopy (Nikon Eclipse Ti). Apoptotic cells were assessed by nuclear staining color (brown) and condensation. The percentage of apoptotic cells was calculated for each field and averaged for the treatment group.

Western blotting. In some experiments, cells were directly lysed in Ripa buffer containing protease inhibitor cocktail (Cat: P8340; 1:100 dilution, Sigma) 48 hours after the transfection. The lysates were directly subjected to SDS-PAGE followed by western blotting with antibody against Klotho (AF1819, R&D Systems). The blot was rinsed and reprobed with antibody against β-actin as the loading control. In other experiments, transfected cells were seeded on collage IV-coated dishes and incubated in DMEM containing 10% BSA for 24 hours. These cells were incubated in serum-free medium overnight and then treated with 1 mM STZ, 40 ng/mL TNFα, IgG1 (5 μg/mL), or integrin β1 blocking antibody (5 μg/mL) for 24 hours. Cells were lysed in Ripa buffer containing protease inhibitor cocktail (2.5 mM sodium pyrophosphate, 1 mM β-glycerolphosphate, 2 mM sodium vanadate, 1 mM EDTA, and 1 mM EGTA). The protein concentration was measured with Pierce BCA assay (Thermo Scientific). Lysates (40 μg protein/well) under the reducing condition were directly subjected to SDS-PAGE (4-20% Tris-HCL precast gel, Bio-Rad) followed by Western blotting with antibody against p-Akt (Ser 473) (Cat: 4051, Cell Signaling Technology), p-FAK (Y397) (Cat: ab4803, Abcam), or cleaved Caspase 3 (Cat: 9664, Cell Signaling Technology), respectively. The same blot was re-probed with antibodies against Akt (Cat: 4685, Cell Signaling Technology), FAK (Cat: ab72140, Abcam), or Caspase 3 (Cat: 9662, Cell Signaling Technology) after stripping the blot, respectively. The same blot was stripped again and reprobed with antibody against f-actin as the loading control.

Immunoprecipitation. Transfected cells were lysed with Ripa buffer containing protease inhibitor cocktail. Lysates (500 μg/250 μL) were incubated with 1 μg of integrin β1 antibody (cat: 610467; BD Biosciences) for overnight at 4° C. Lysates were then incubated with 50 μl of 50% Protein-A beads (Cat: 16-125; Millipore) for 2 hours. Immune complexes were pelleted and washed with Ripa buffer 5 times. After the final wash, each pellet was resuspended in 100 μl of 2× Laemmli sample buffer containing β-mercaptoethanol and boiled for 5 min. Samples were subsequently analyzed through western blotting.

Statistical Analysis. All data were expressed as mean±SEM. Blood glucose was analyzed by a repeated measures one-way ANOVA. The remaining data were analyzed by a one-way ANOVA. The Newman-Keuls procedure was used to reveal differences between groups. A probability value with p<0.05 was considered to be statistically significant.

Results of Example 2:

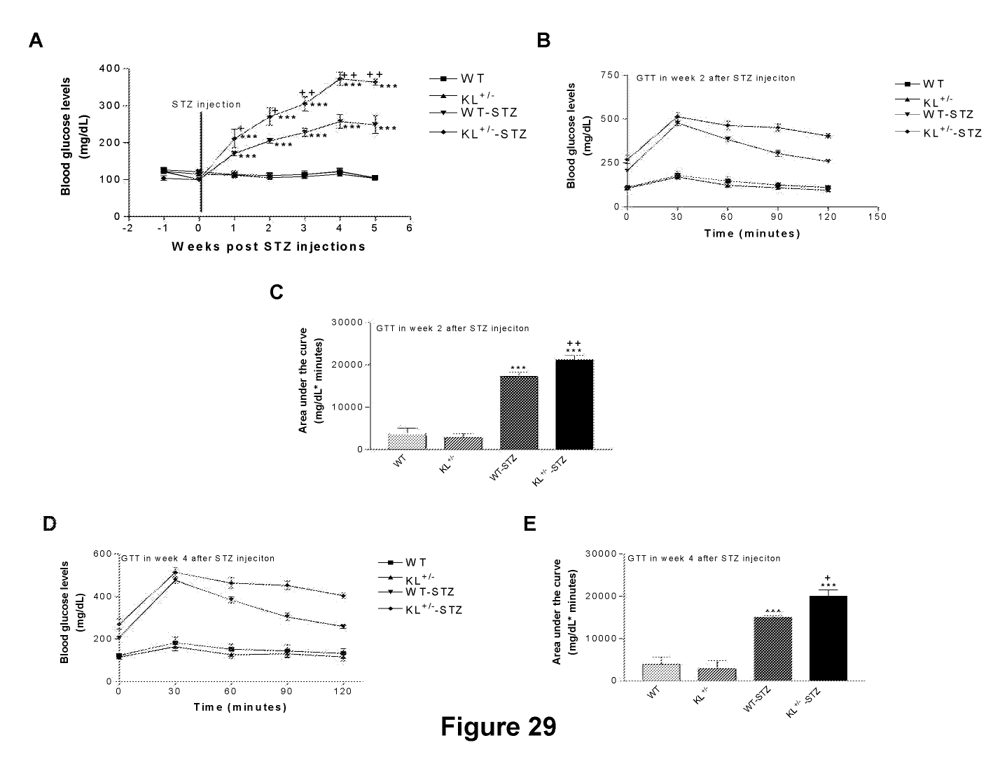

Half deficiency of Klotho in KL^{+/-} mice exacerbated the development of T1DM induced by STZ. To study the roles of endogenous Klotho in the development of T1DM, KL^{+/-} and their litter mate WT mice treated with multiple low doses of STZ were used. STZ caused hyperglycemia in both KL^{+/-} and WT mice after 1-week treatment with STZ (FIG. 29). Notably, KL^{+/-} mice demonstrated severer hyperglycemia vs. the WT mice (FIG. 29), indicating that half deficiency of Klotho exacerbated T1DM in response to the STZ challenge.

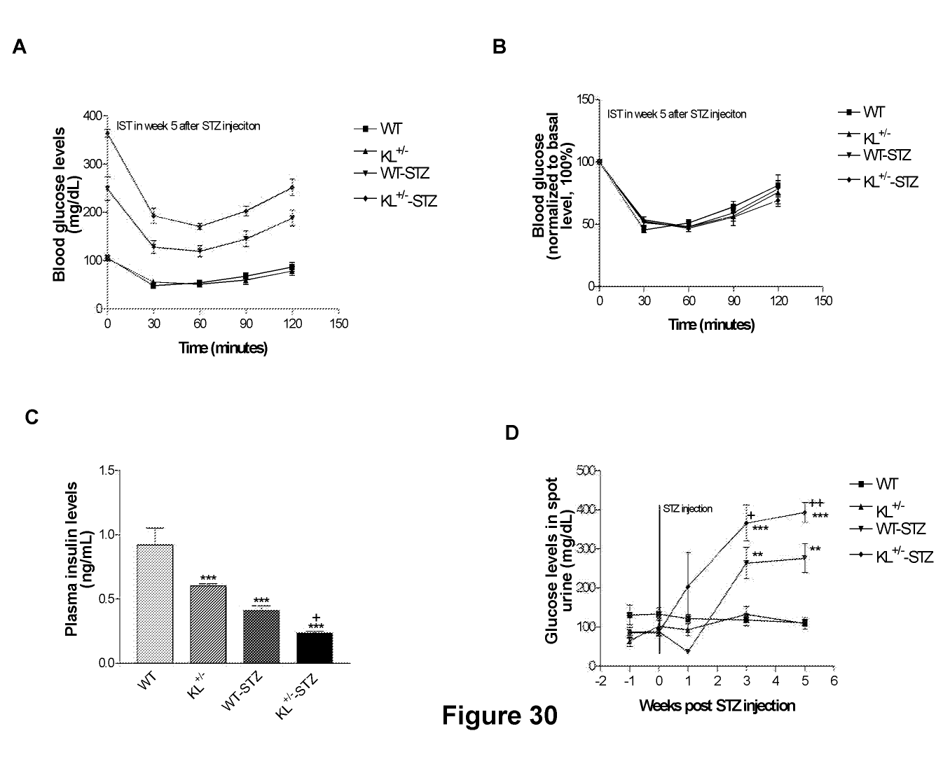

To gain insights for the mechanism of Klotho action, the subcutaneous glucose tolerance test (GTT) was examined at weeks 2 and 4 and subcutaneous insulin sensitivity test (IST) at week 5 after the initial injection of STZ. Indeed, KL^{+/-} and WT mice displayed glucose intolerance after STZ challenge. KL^{+/-} mice demonstrated increased glucose intolerance vs. the wild type mice (FIG. 29b-e), suggesting that half Klotho deficiency exacerbated β cell damage in response to the STZ challenge. KL^{+/-} and wild type mice treated with STZ did not show significant difference in insulin sensitivity test (FIG. 30a-b). Thus, these data indicate that half deficiency of Klotho may directly impair pancreatic β cell function rather than affect insulin sensitivity in STZ-induced diabetic mice.

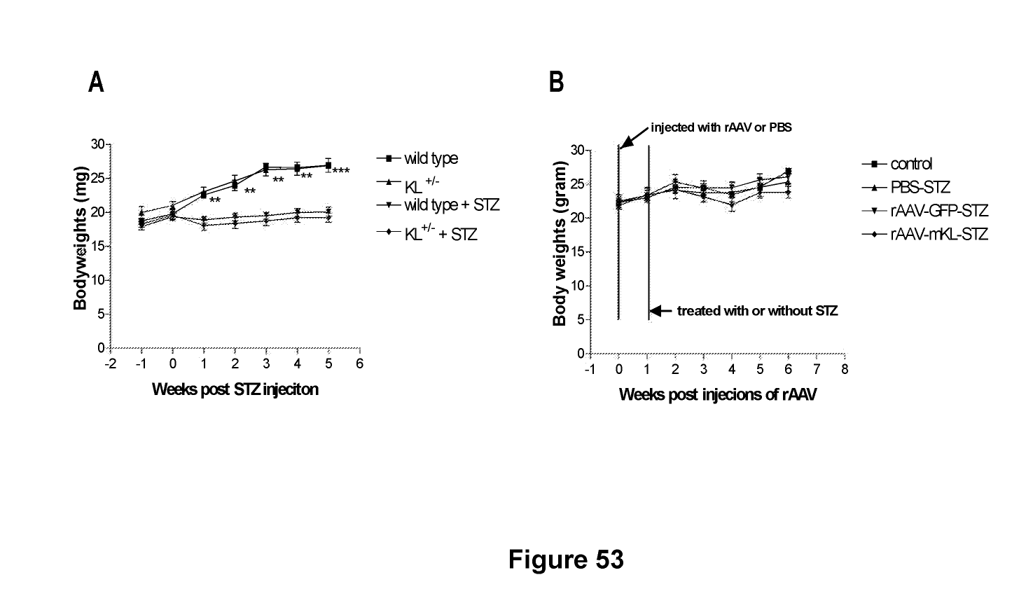

In addition, the basal plasma Insulin levels in KL^{+/-} mice were significantly lower than those of wild type mice (FIG. 30c). Both KL^{+/-} and wild type mice displayed lower plasma levels of insulin after the STZ challenge (FIG. 30c). KL^{+/-} mice demonstrated lower insulin levels vs. WT mice after STZ treatments (FIG. 30c). Fasting urine glucose levels were significantly higher in KL^{+/-} mice vs. WT mice treated with STZ (FIG. 30d). STZ decreased body weights in both WT and KL^{+/-} mice (FIG. 53a). However, the genotype difference did not alter body weight after the injection of STZ (FIG. 53a). Therefore, endogenous Klotho deficiency exacerbated STZ-induced diabetes.

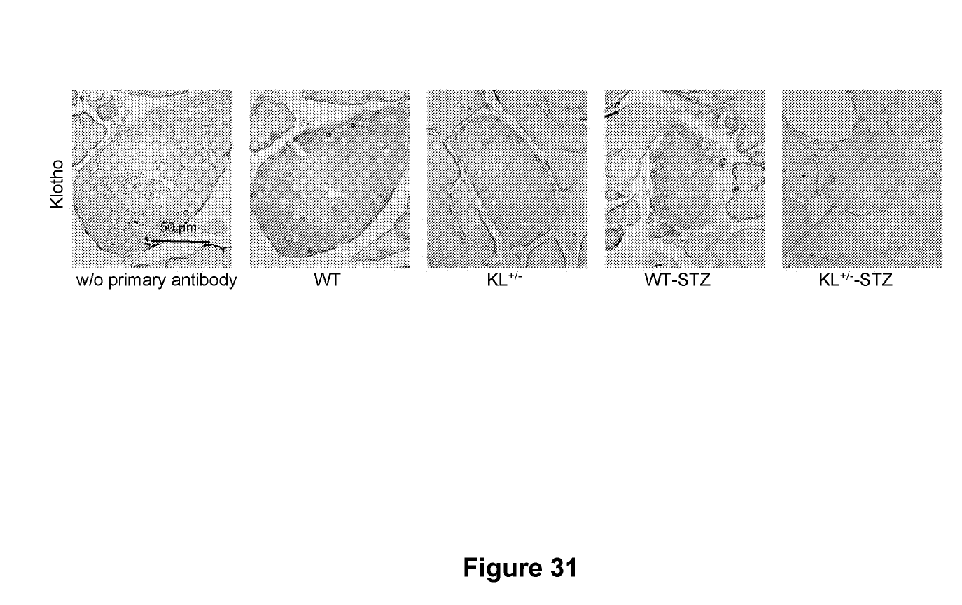

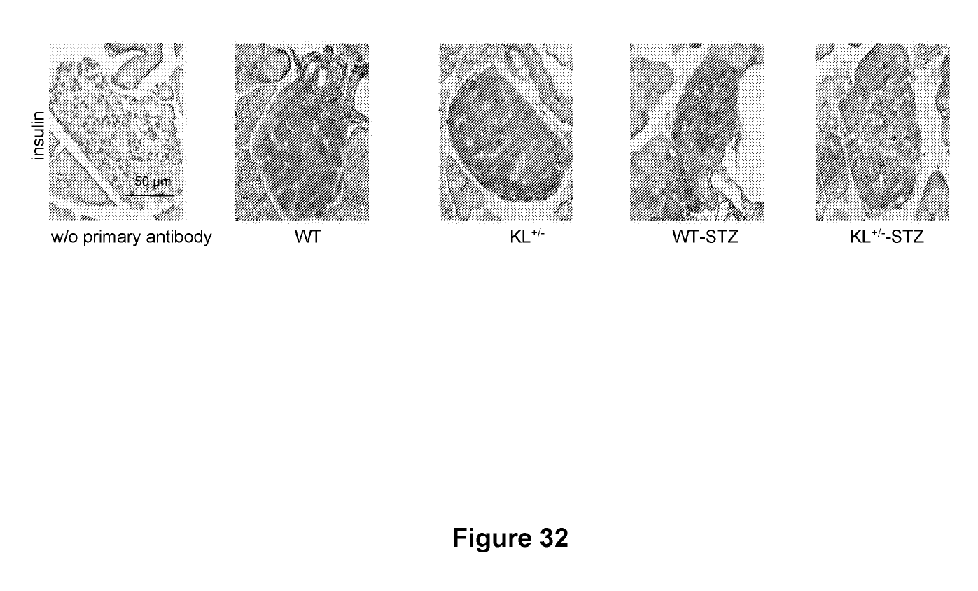

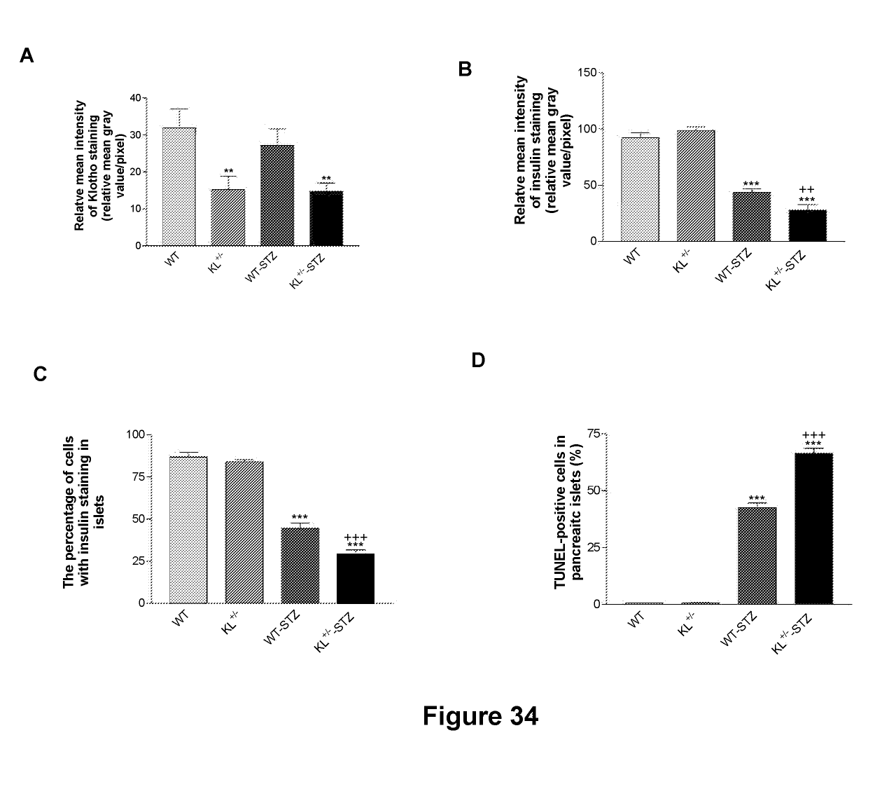

Half deficiency of Klotho in KL^{+/-} mice exacerbated STZ-induced cell apoptosis and depletion. IHC analysis indicated that Klotho staining in pancreatic islets of Langerhans was significant lower in KL^{+/-} mice vs. WT mice (FIG. 31, FIG. 34a). STZ did not alter Klotho staining. There was no significant difference in insulin staining in pancreatic islets between the wild type and KL^{+/-} mice without STZ treatments (FIG. 32, FIG. 34b). STZ decreased insulin staining in islets of WT and KL^{+/-} mice. Interestingly, insulin staining was significantly lower in KL^{+/-} mice than in WT mice treated with STZ (FIG. 32, FIG. 34b). In addition, STZ caused a significant drop in the number of insulin-positive cells in pancreatic islets in both strains. STZ resulted in a greater drop in the number of insulin-positive cells in pancreatic islets in KL^{+/-} mice than in WT mice (FIG. 32, FIG. 34c). Therefore, Klotho deficiency predisposed mice to STZ-induced insulin depletion in pancreatic β cells.

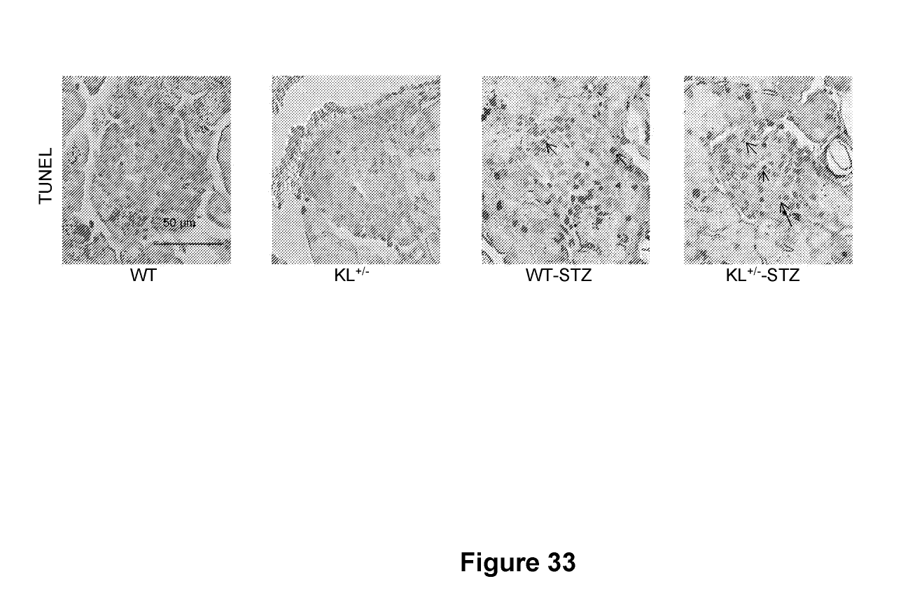

STZ caused an increase in the number of apoptotic cells in pancreatic islets in both strains (FIG. 33, FIG. 34D). Notably, STZ led to a greater increase in the number of apoptotic cells in pancreatic islets in KL^{+/-} mice vs. WT mice (FIG. 33, FIG. 34D). Therefore, half deficiency of Klotho predisposed pancreatic islet cells to STZ-induced apoptotic stimuli.

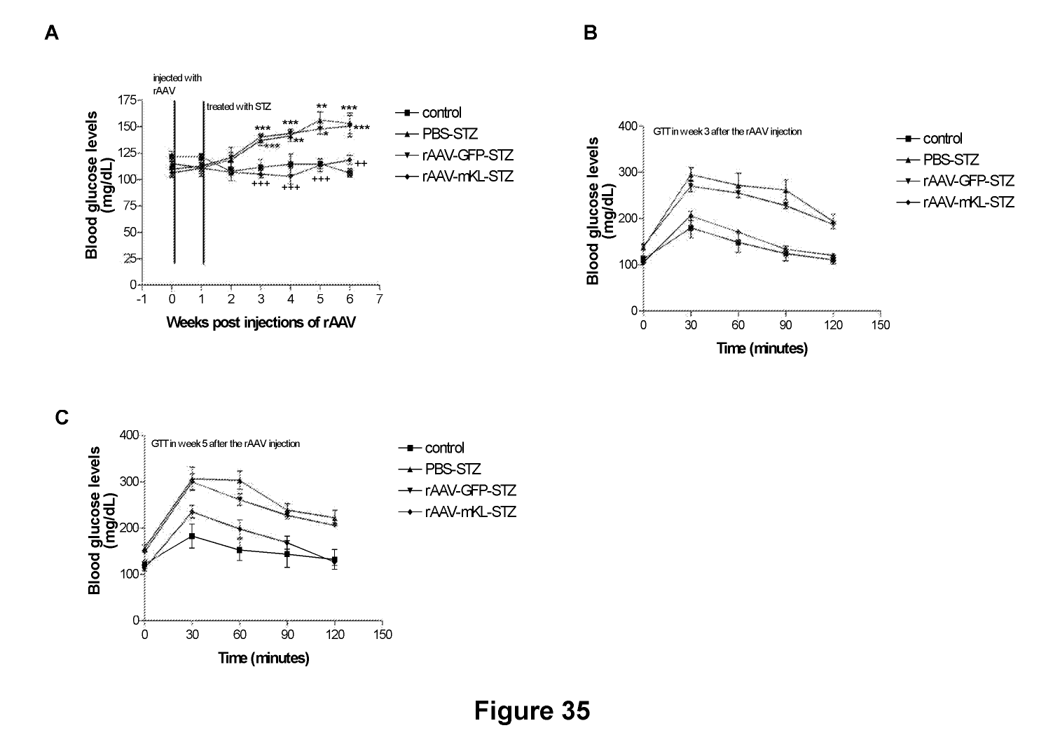

β cell-specific expression of mKL attenuated the development of hyperglycemia in mice challenged with STZ. To test whether β cell-specific expression of mKL attenuates hyperglycemia induced by STZ, PBS, rAAV-GFP, or rAAV-mKL were injected into 10 weeks old male WT mice (129S1/Svlm) at the region of pancreas via IP delivery. One week later, these mice were injected with multiple low doses of STZ or citrate buffer (control). As shown in FIG. 35a, injections of STZ caused mild hyperglycemia in these 129S1/Svlm mice. In contrast, rAAV-mKL prevented the development of mild hyperglycemia induced by STZ.

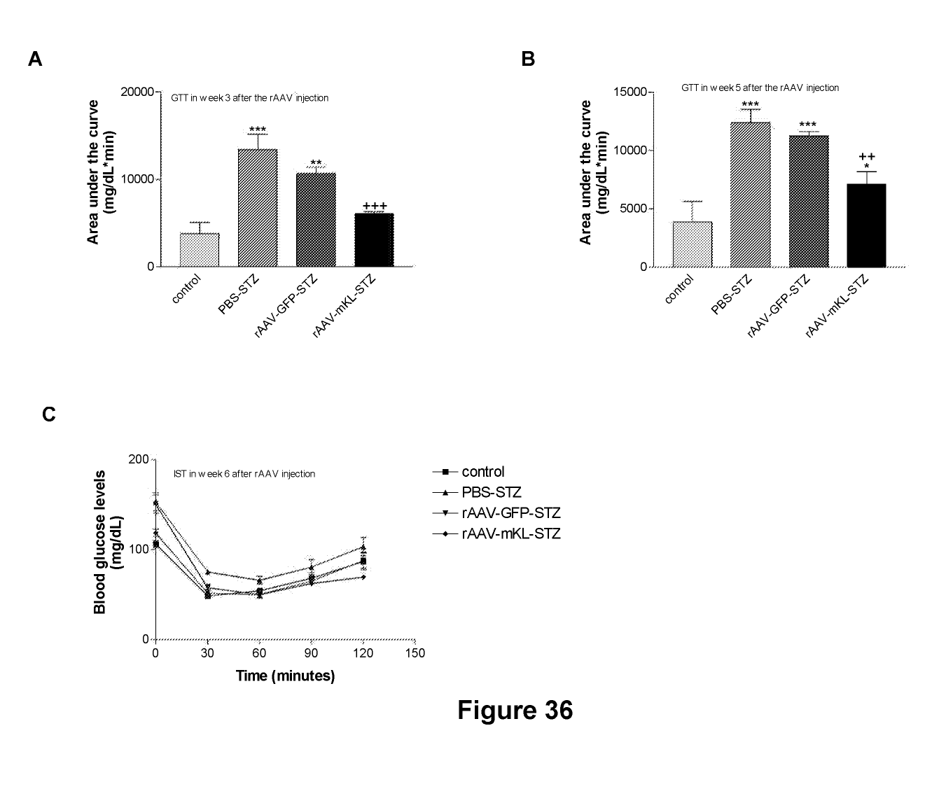

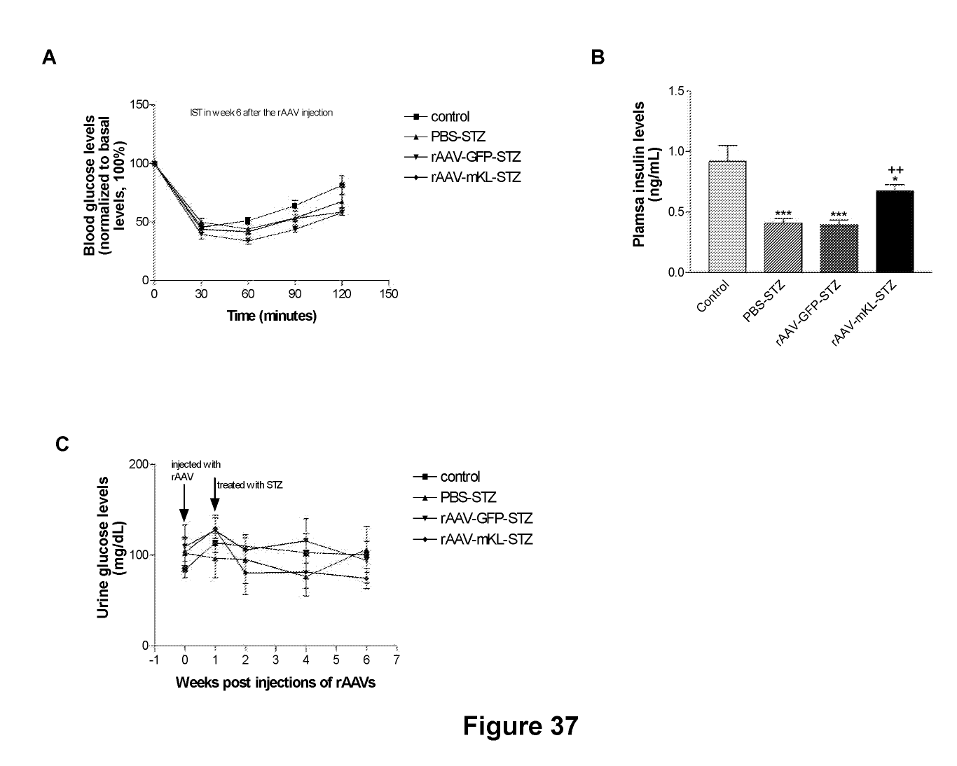

STZ caused glucose intolerance vs the control group (FIG. 35b-c, FIG. 36a-b). However, the treatment of rAAV-mKL prevented glucose intolerance compared to the PBS and rAAV-GFP-treated control groups (FIG. 35b-c, FIG. 36a-b). Injections of rAAV or STZ did not alter insulin sensitivity in 129S1/Svlm mice (FIG. 36c, FIG. 37a). Therefore, rAAV-mKL prevented STZ-induced hyperglycemia and improved glucose tolerance via preserving pancreatic β cell function.

In addition, the STZ challenge caused a decrease in plasma insulin levels (FIG. 37b). In contrast, the treatments with rAAV-mKL attenuated the decrease in insulin levels (FIG. 37b). Thus, rAAV-mKL may increase insulin release in 129S1/Svlm mice. No significant change in urine glucose was found in these mice (FIG. 37c). Injection of rAAV or STZ did not alter body weights in these 129S1/Svlm mice (FIG. 53b). Therefore, β cell-specific expression of mKL prevented hyperglycemia and glucose intolerance in STZ-treated animals.

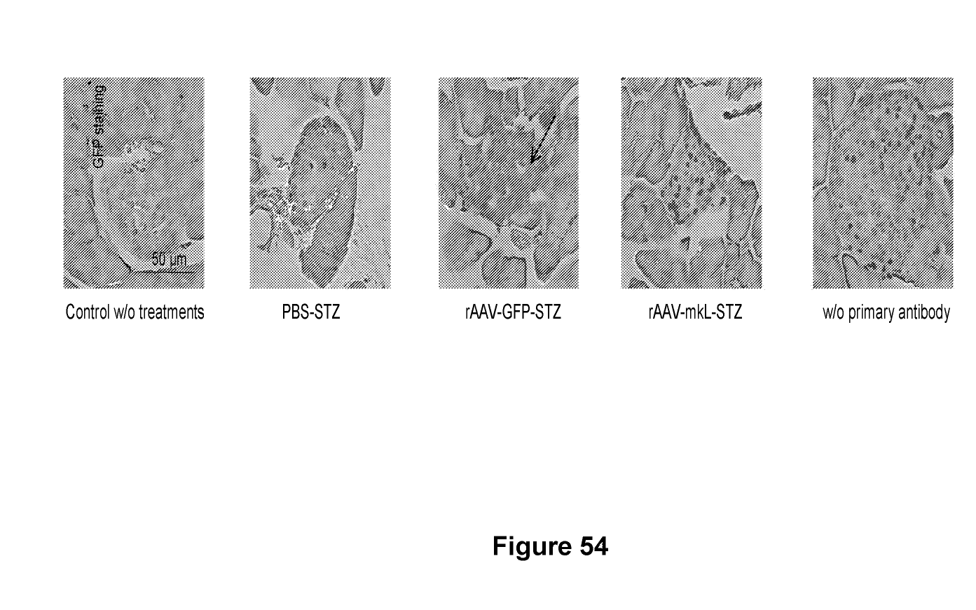

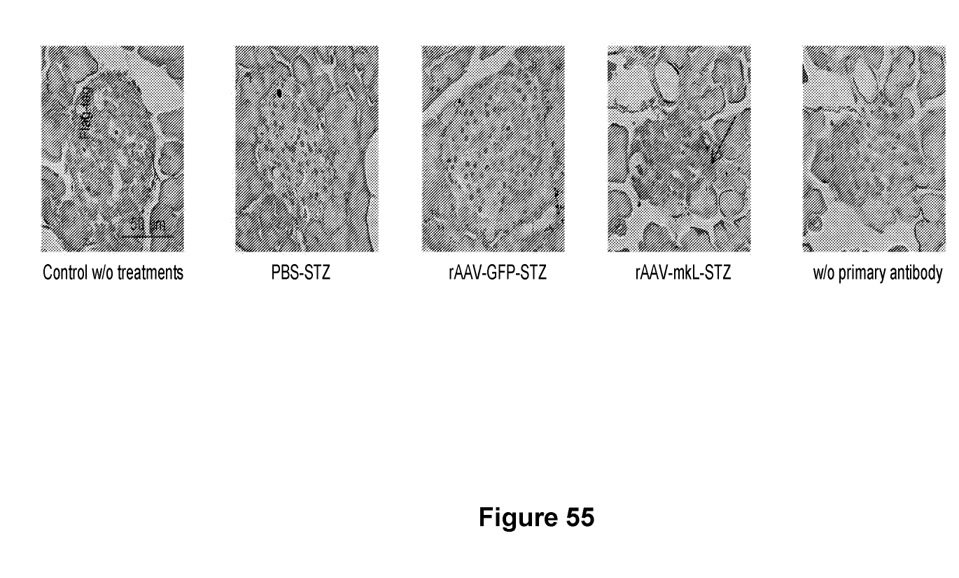

β cell-specific expression of mKL prevented apoptosis in pancreatic islet cells in STZ-treated mice. Different serotypes of rAAV with single or double-stranded DNA have been used in pancreatic islet gene transfer with various efficiencies via different routes (5,6). Given that the insert genes ranged from 0.7 to 3.1 kb, AAV2 vector with single stranded DNA was used in the gene transfer. A Flog-tog sequence had been inserted at 3′ end of mouse Klotho gene. Six weeks after the injections, GFP protein and FLAG-tag were first examined in cross-sections of paraffin-embedded pancreatic islets using IHC. Obviously, the intraperitoneal deliveries of rAAV-GFP or rAAV-mKL drove GFP or FLAG-tag expression in pancreatic islets of 129S1/Svlm mice (FIG. 54, FIG. 55). Thus, intraperitoneal delivery of rAAV coupled with mouse insulin II promoter led to β-specific gene expression in mice.

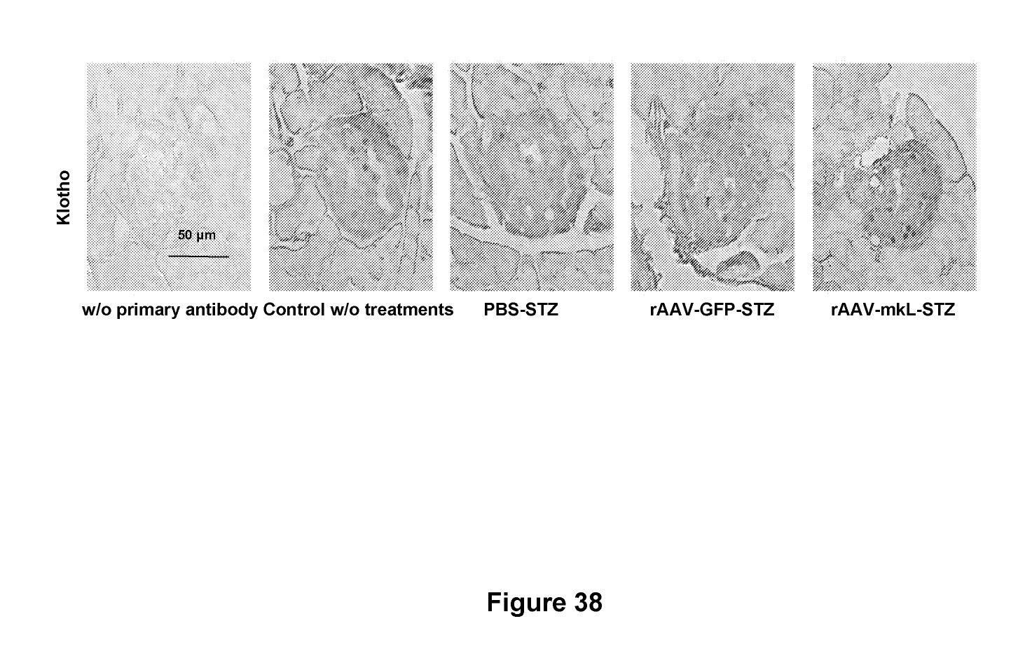

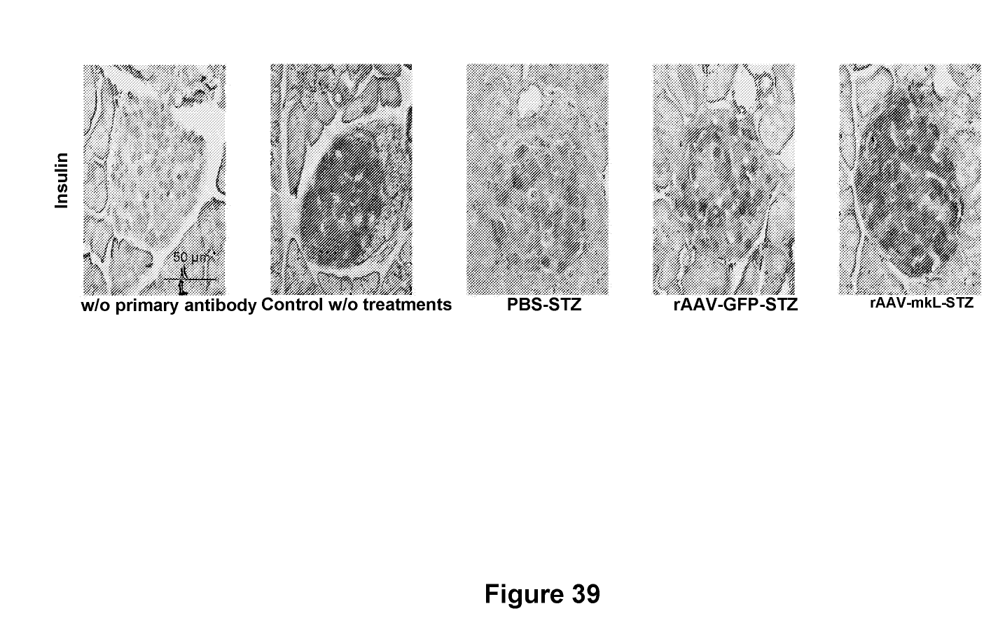

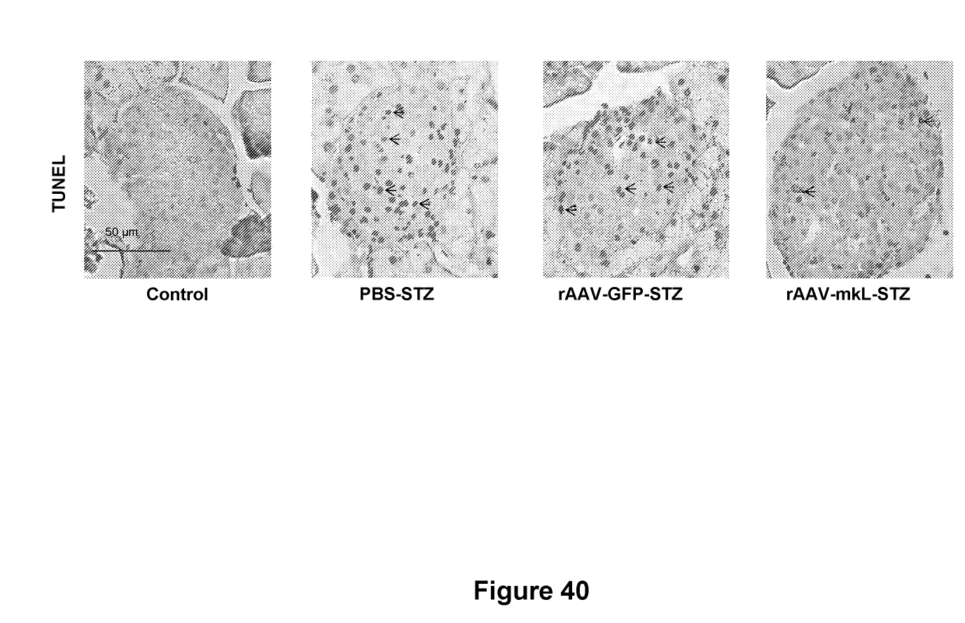

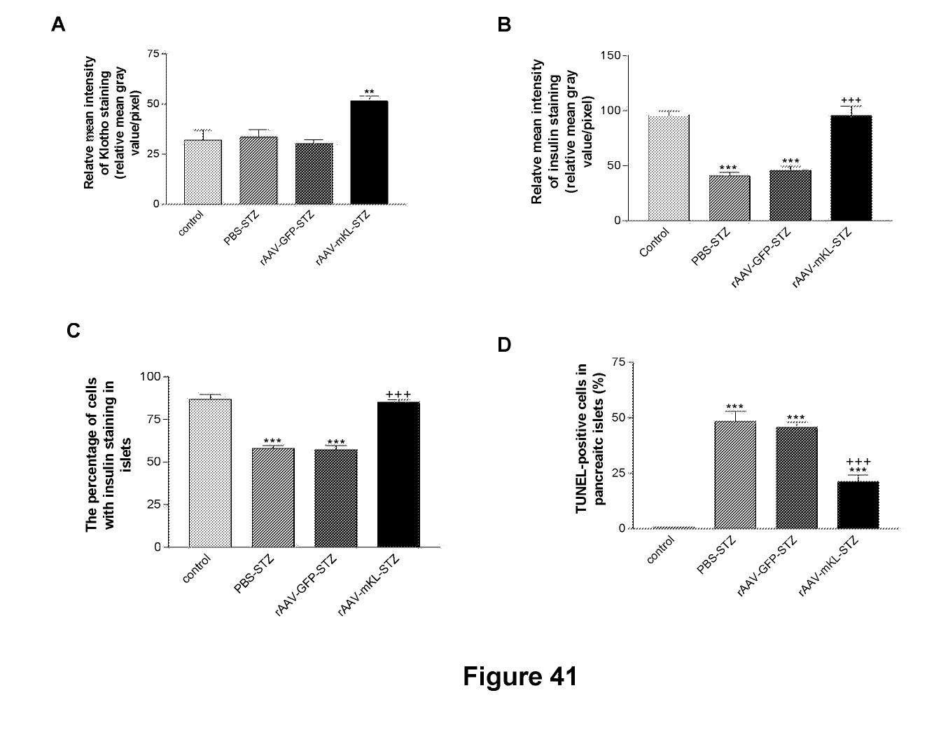

The IHC analysis indicated that treatments with rAAV-mKL increased Klotho protein staining in pancreatic islets (FIG. 38, FIG. 41a), suggesting effective gene delivery. In addition, STZ did not alter Klotho expression in pancreatic islets (FIG. 38, FIG. 41a). STZ decreased insulin staining and the number β cells in pancreatic islets (FIG. 39, FIG. 41b-c). In contrast, β cell-specific expression of mKL prevented the STZ-induced decreases in insulin staining (storage) and the number of β cells in pancreatic islet (FIG. 4C-E). Therefore, β cell-specific expression of mKL preserved pancreatic β cell function and prevented the insulin depletion in pancreatic islets in STZ-treated mice. STZ increased apoptosis in pancreatic islets of 129S1/Svlm mice (FIG. 40, FIG. 41d). In contrast, β cell-specific expression of mKL protected against STZ-induced apoptosis in pancreatic islets (FIG. 40, FIG. 41d). Therefore, β cell-specific expression of mKL prevented STZ-induced β cell failure and apoptosis in pancreatic islets.

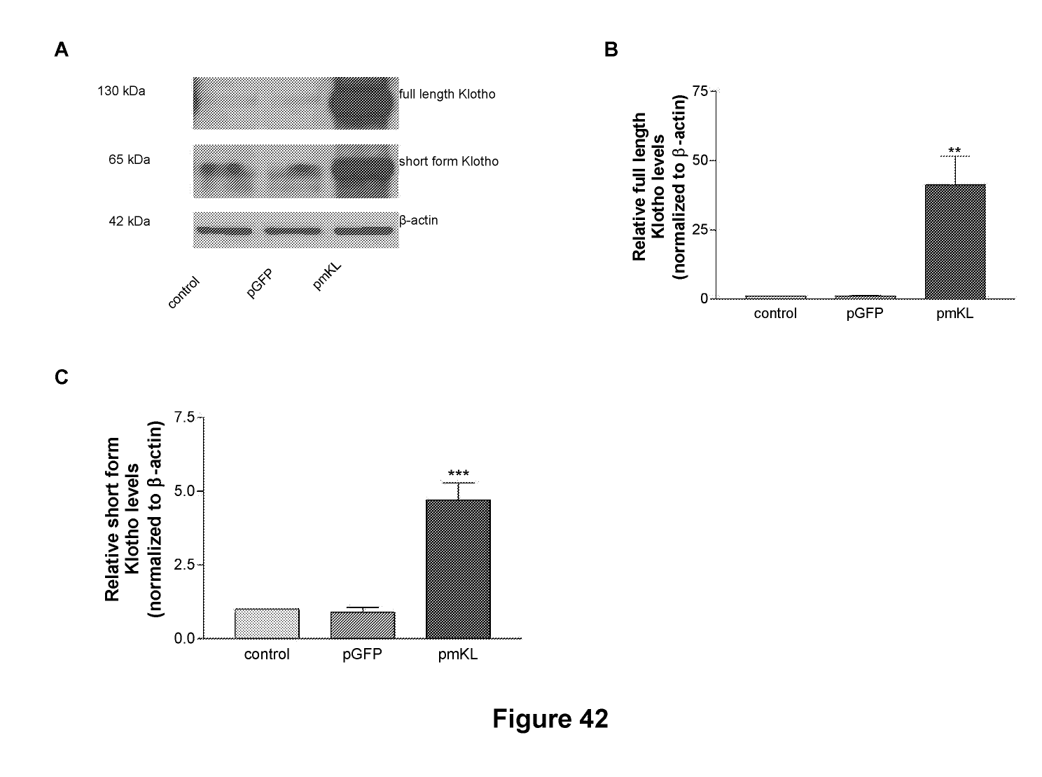

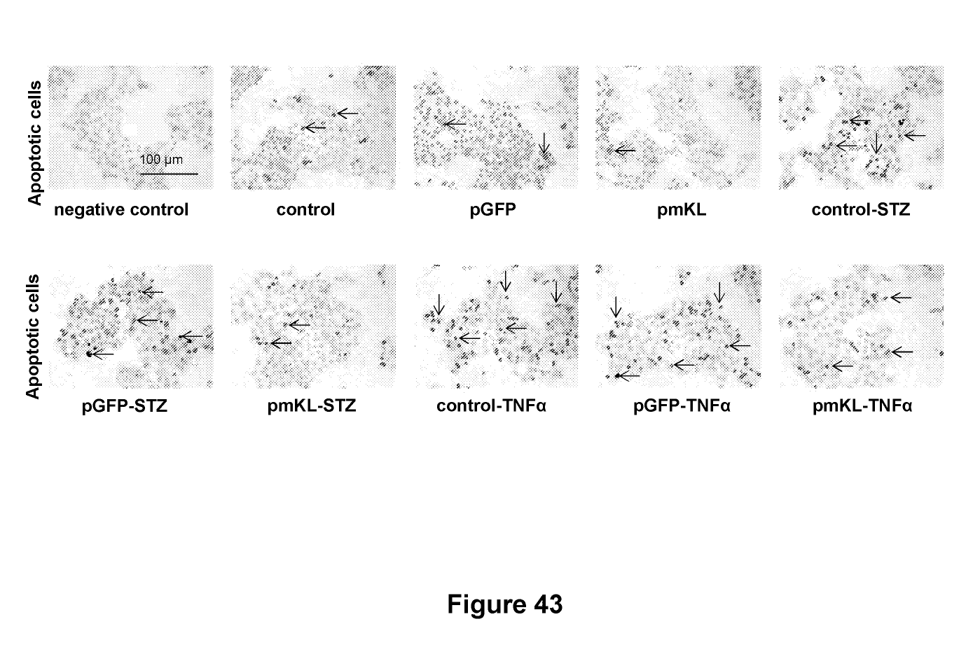

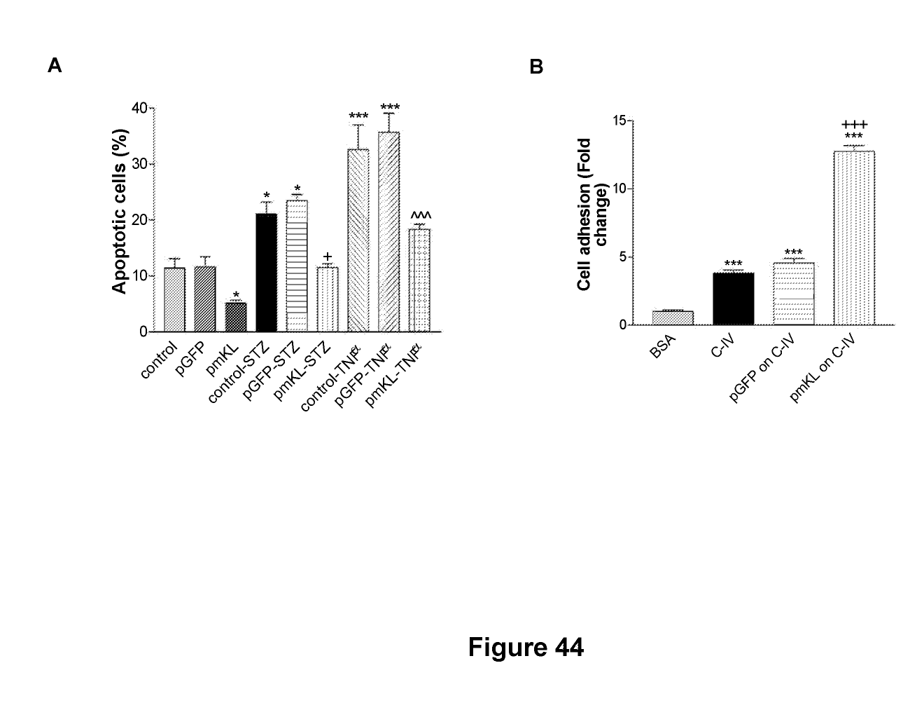

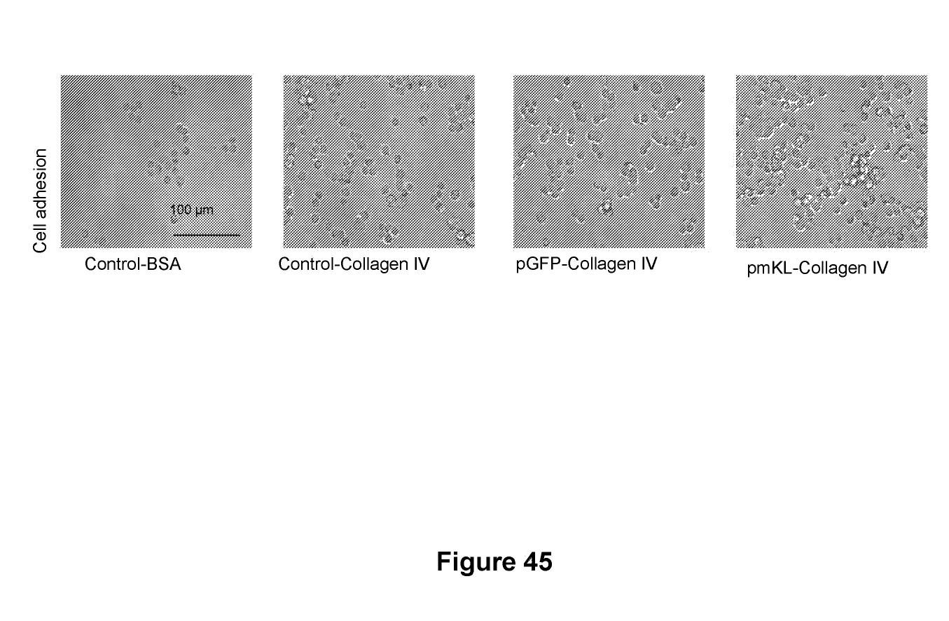

Expression of Klotho attenuated basal, STZ- and TNFα-induced apoptosis, respectively, and increased cell adhesion to collagen IV in MIN6 β cells. The molecular mechanism underlying the beneficial effects of Klotho was further studied on pancreatic 1 cells using MIN6 β cells. STZ and TNFα have been shown to cause apoptosis in MIN β cells (7). Transfection of cells with plasmid DNA pmKL for 48 hours increased Klotho protein in MIN6 β cells (FIG. 42). Interestingly, expression of mKL attenuated the basal, STZ-induced, and TNFα-induced apoptosis, respectively, in MIN6 β cells as demonstrated by TUNEL staining (FIG. 43, FIG. 44a). In addition, expression of mKL enhanced collagen IV-induced cell adhesion (FIG. 45, FIG. 44b). Therefore, Klotho protected against apoptosis and promoted cell adhesion to collagen IV in MIN6 β cells.

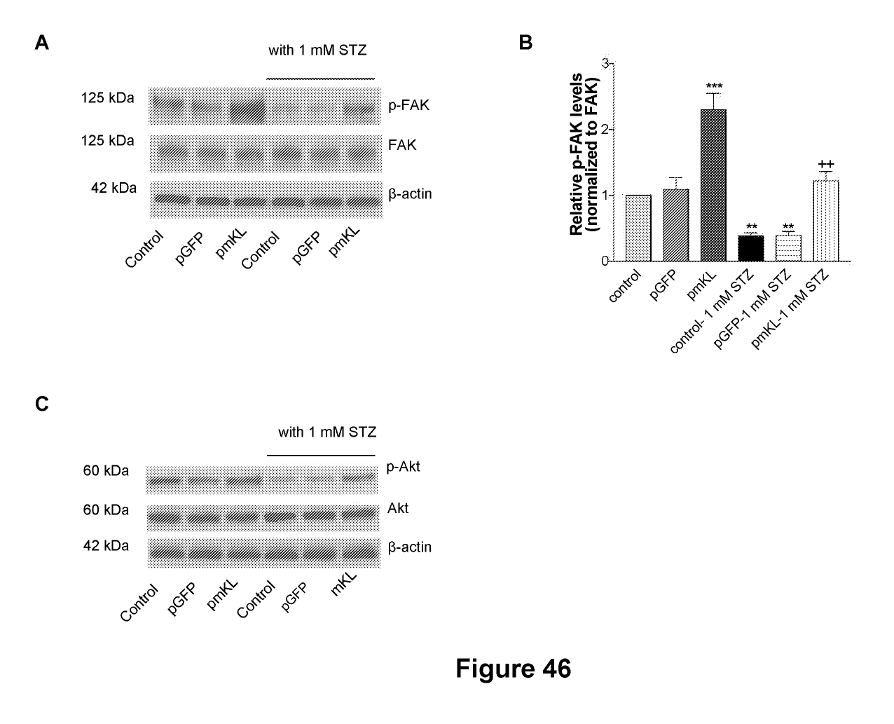

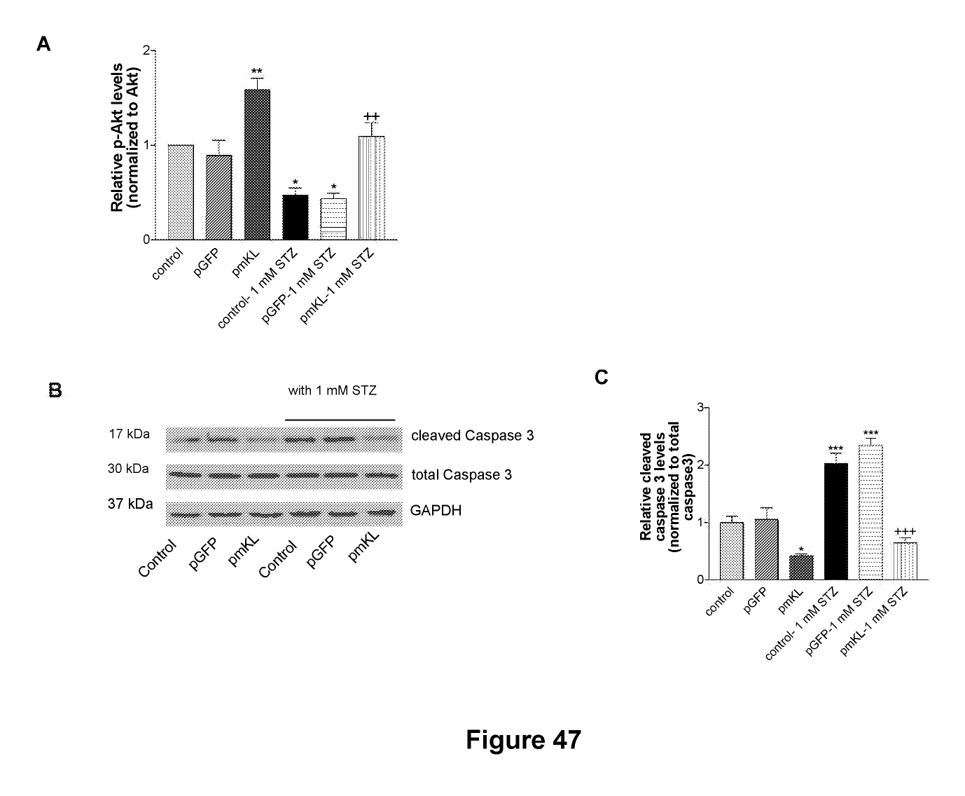

Expression of Klotho promoted phosphorylations of FAK and Akt and decreased Caspase 3 cleavage in MIN6 β cells. It was found that expression of mKL increased phosphorylation of FAK and abolished the STZ-induced inhibition of phosphorylation of FAK in MIN6 β cells (FIG. 46a-b). In addition, expression of mKL increased phosphorylation of Akt and abolished STZ-induced inhibition of phosphorylation of Akt in MIN6 cells (FIG. 46c, FIG. 47a). Furthermore, expression of mKL decreased Caspase 3 cleavage and prevented STZ-induced Caspase 3 cleavage in MIN6 β cells (FIG. 47b-c). Therefore, the actions of Klotho involve upregulation of phosphorylation of FAK, an adhesion signaling and phosphorylation of Akt, a survival signaling as well as the inhibition of Caspase 3 activity, a general apoptotic pathway.

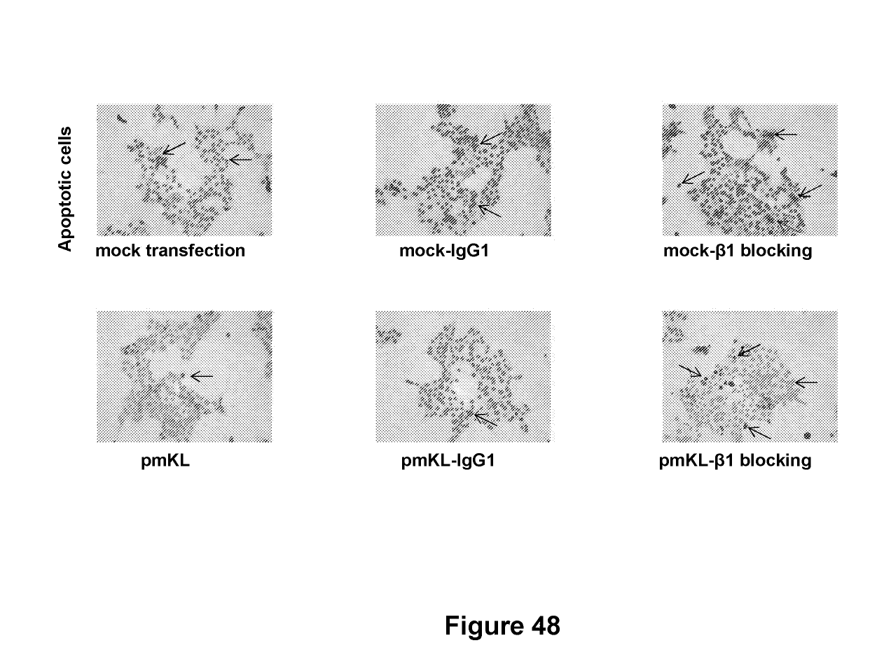

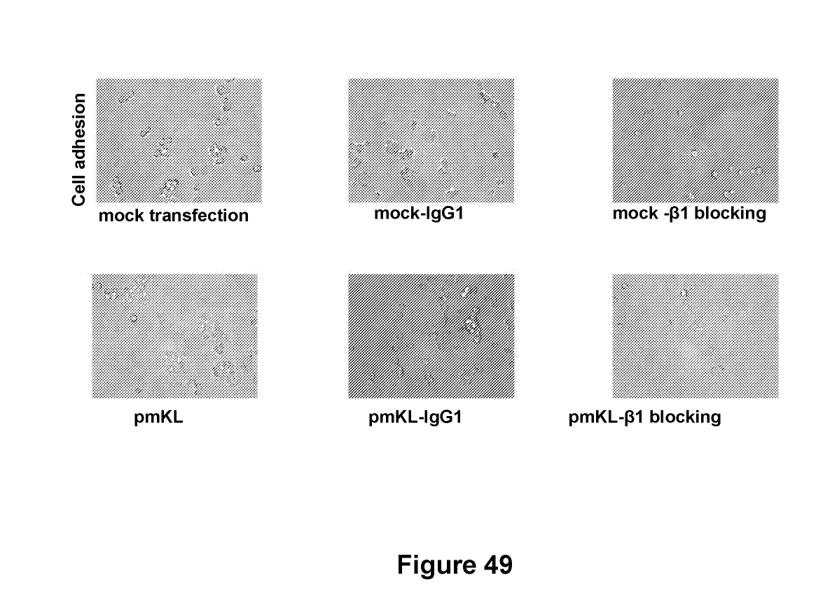

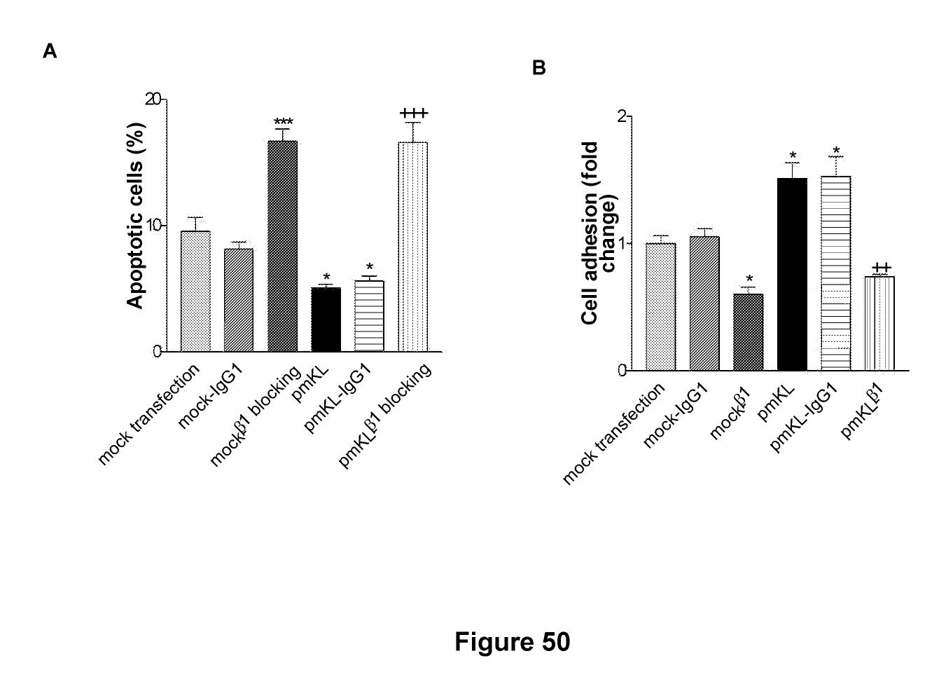

Integrin β1 was required for protecting against apoptosis and promoting cell adhesion by Klotho. Next it was investigated whether integrin β1 subunit, which acts as a receptor for extracellular matrix including collagen IV with various integrin a subunits, was required for β cell response to Klotho expression. Integrin 01 is expressed in mouse pancreatic islet cells and MIN6 cells. Blocking of integrin β1 using integrin β1 blocking antibody abolished the protection against apoptosis by Klotho in MIN6 cells (FIG. 48, FIG. 50a). In addition, blocking of integrin β1 attenuated Klotho-promoted cell adhesion to collagen IV (FIG. 49, FIG. 50b). Therefore, integrin β1 was required for inhibiting apoptosis and promoting cell adhesion by Klotho in MIN6 β cells.

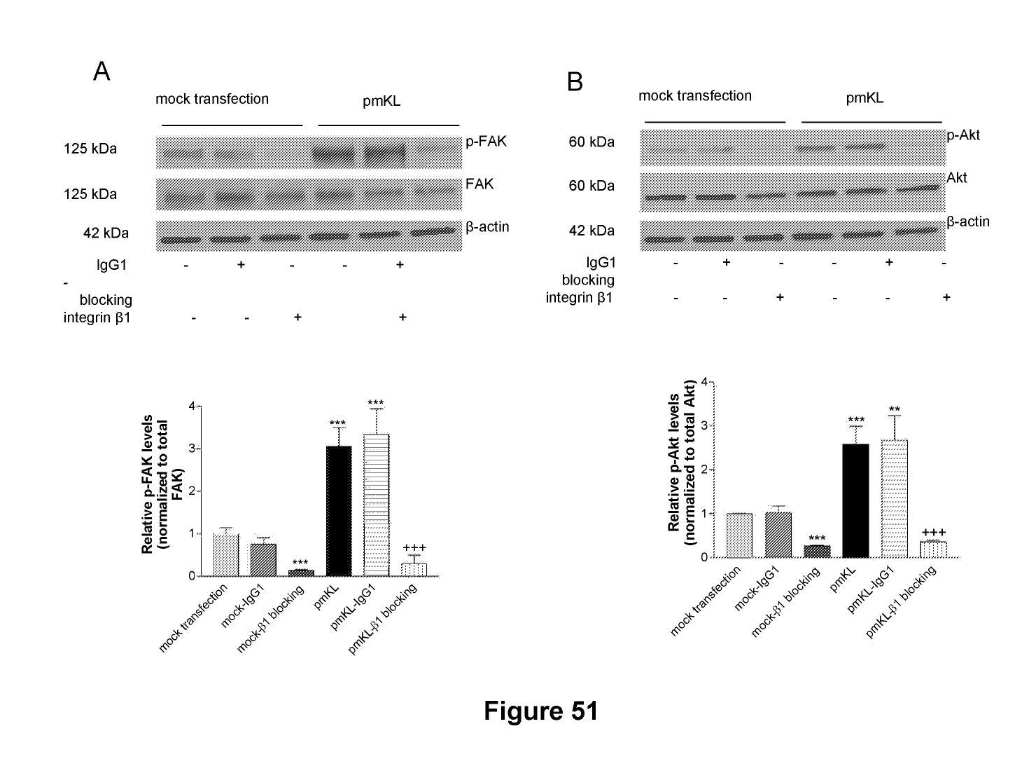

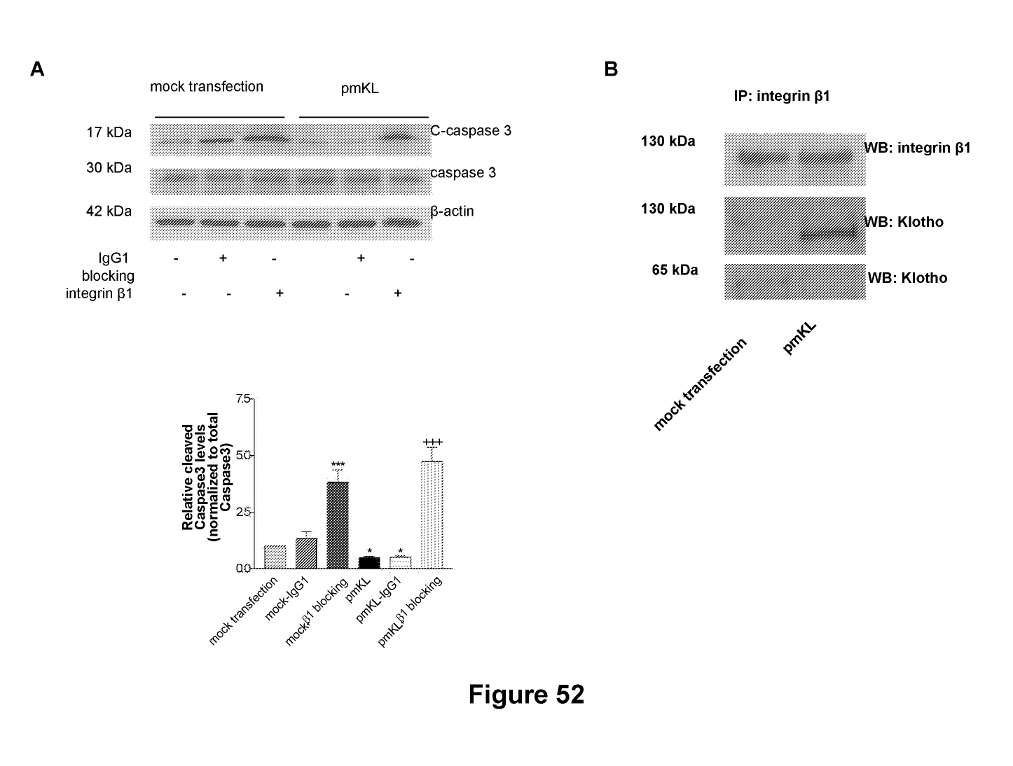

Integrin β1 was required for upregulating phosphorylation of FAK and Akt and inhibiting Caspase 3 cleavage by Klotho. It was found that blocking of integrin β1 attenuated Klotho-promoted phosphorylations of FAK and Akt in MIN6 cells (FIG. 51). Furthermore, blocking of integrin β1 abolished the inhibition of Caspase 3 cleavage by Klotho (FIG. 52a). Thus, integrin β1 is required for upregulating phosphorylations of FAK and Akt and inhibiting Caspase 3 cleavage by Klotho. Interestingly, the co-immunoprecipitation study revealed that Klotho was co-immunoprecipitated with integrin β1, suggesting that there is a physical association between Klotho and integrin β1 (FIG. 52b).

Discussion of Example 2:

The present work demonstrated, for the first time, that half deficiency of Klotho in pancreatic islets predisposes mice to insulin-deficient diabetes induced by STZ. STZ-induced hyperglycemia, glucose intolerance, pancreatic islet apoptosis, and decreases in pancreatic insulin storage and plasma insulin levels were significantly exacerbated by deficiency of Klotho (FIGS. 29-30). As indicated above, knockdown of Klotho by siRNA was found to significantly attenuate glucose-induced insulin secretion in MIN6 β cells. Therefore, endogenous Klotho appears to be involved in the regulation of the physiological functions of β cells. Notably, KL deficiency makes β cells more susceptible to apoptotic stimuli (STZ) and predispose to β cell dysfunction (FIGS. 29-34). Interestingly, β cell-specific expression of Klotho preserved pancreatic β cells from apoptosis, attenuated hyperglycemia, and enhanced the performance in glucose tolerance in STZ-treated mice (FIGS. 35-41). Therefore, overexpression of Klotho in pancreatic β cells is sufficient to preserve the physiological functions of β cells in response to STZ challenge.

A novel finding of the present work is that expression of mKL promoted cell adhesion to collagen IV, abolished STZ-induced inhibition of FAK and Akt, and attenuated STZ-induced Caspase 3 cleavage and apoptosis in MIN6 β cells (FIGS. 42-47). Furthermore, it was found that blocking of integrin β1 subunit abolished the promoting effects of Klotho on cell adhesion and phosphorylations of FAK and Akt as well as the protective effects of Klotho against apoptosis in MIN6 β cells (FIGS. 48-52). Therefore, without wishing to be bound by theory, this study reveals a new molecular pathway that mediates the beneficial effects of Klotho in β cells, i.e., integrin β1-FAK/Akt-Caspase 3.

Several lines of evidence support these findings. The extracellular matrix (ECM) stimulates a plethora of cellular processes, including cell differentiation, proliferation, survival, and function by interacting with classical cell adhesion receptors, the integrins. Integrins are heterodimeric transmembrane molecules composed of distinct α and β subunits. Integrins have unique bidirectional signaling properties that incorporate the external and internal environments of a cell, allowing for dynamic coordination of extracellular events with intracellular changes. The major components of ECM in pancreatic islets are collagen IV, laminins, heparan sulfate proteoglycans such as perlecan. In isolated human islets, collagen IV has been shown to upregulate insulin secretion and β cell survival. In vitro, collagen IV has been found to prevent MIN6 β cells from apoptosis. In mouse pancreatic islets, heterodimers containing β1-integrin have been suggested to affect insulin transcription and secretion as well as β-cell proliferation. Integrin β1 has been shown to regulate rat β cells (INS-1) survival (70). Male mice with a conditional knockout of β1-integrin in collagen I-producing cells, display impaired glucose tolerance, with a significant reduction in pancreatic insulin contents and in β cell mass. In addition, FAK activation has been shown to be involved in glucose stimulated insulin secretion in MIN6 β cells, rat primary β cells, or isolated islets. Most recent studies have shown that pancreatic β-cell-specific deletion of FAK attenuated β cell viability and function in transgenic mice.

Furthermore, Akt is believed to play important roles in the growth β-cells and the protection of β cells from apoptotic stimuli. Finally, Caspase 3 knockout mice were protected from STZ-induced β cell apoptosis and diabetes. Integrin β1 may be an important upstream mediator of Klotho-induced activation of FAK/Akt and inhibition of Caspase 3 cleavage which can be abolished by blocking of integrin β1 (FIGS. 51-52). Therefore, Klotho protects β cells from apoptosis and preserves β cell function by activation of FAK and Akt and inhibition of Caspase 3 via interacting with integrin β1.

It has been reported that Klotho exhibited weak β-glucuronidase activity in vitro. Klotho participated in removal of α2,6-linked sialic acids of TRPV5 through its sialidase activity in CHO cells. Removal of terminal sialic acids from N-glycans exposes underlying LacNAc for binding with galectin-1 leading to enhanced retention of TRPV5 at the cell surface. Based on the amino acid sequences, integrin β1 contains twelve potential asparagine-linked glycosylation sites and desialylation of α5β1 integrin leads to increased adhesion to fibronectin, affecting cell adhesion in myeloid cells. Interestingly, it was found here that Klotho can be co-immunoprecipitated with integrin β1 (FIG. 52b), suggesting that Klotho may bind to integrin β1. In addition, Klotho also activated the down-steam signaling of integrin β1 (FAK/Akt-Capase 3) in MIN6 cells (FIG. 51-52).

It is noted that KL deficiency decreased the basal plasma insulin level (FIG. 30c) but did not affect insulin storage levels in pancreatic islets (FIG. 32, FIG. 34b-c). Therefore, KL deficiency may impair insulin release rather than affect insulin synthesis. This notion is supported by a recent study that KL enhances glucose-induced insulin release in MIN6 β cells. KL promotes insulin release likely via increasing membrane retention of TRPV2 and intracellular levels of Ca²⁺.

In conclusion, this work established for the first time that Klotho prevents streptozotocin-induced diabetes and protects pancreatic β cells from apoptotic stimuli. Hence, the β cell-specific expression of Klotho, as shown elsewhere herein, finds an application in the inhibition and treatment of T1DM and of other diabetic-related conditions. The beneficial effects of Klotho in β cells, without wishing to be bound by theory, are likely mediated via enhancing the integrin β1-FAK/Akt pathway that inhibits Caspase 3 cleavage. All of the assay methods listed herein are well within the ability of one of ordinary skill in the art given the teachings provided herein.

While the presently disclosed inventive concepts are described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be the most useful and readily understood description of procedures as well as of the principles and conceptual aspects of the presently disclosed inventive concepts. Changes may be made in the formulation of the various compositions described herein or in the steps or the sequence of steps of the methods described herein without departing from the spirit and scope of the presently disclosed inventive concepts.

### CITED REFERENCES

The following references, to the extent that they provide exemplary procedural or other details supplementary to those set forth herein, are expressly incorporated herein by reference in their entireties.
1. Wang Z, Zhu T, Rehman K K, Bertera S, Zhang J, Chen C, Papworth G, Watkins S, Trucco M, Robbins P D, Li J, Xiao X: Widespread and stable pancreatic gene transfer by adeno-associated virus vectors via different routes. Diabetes 55:875-884, 2006
2. Auricchio A, Hildinger M, O'Connor E, Gao G P, Wilson J M: Isolation of highly infectious and pure adeno-associated virus type 2 vectors with a single-step gravity-flow column. Hum Gene Ther 12:71-76, 2001
3. Rohr U P, Wulf M A, Stahn S, Steidl U, Haas R, Kronenwett R: Fast and reliable titration of recombinant adeno-associated virus type-2 using quantitative real-time PCR. J Virol Methods 106:81-88, 2002
4. Veldwijk M R, Topaly J, Laufs S, Hengge U R, Wenz F, Zeller W J, Fruehauf S: Development and optimization of a real-time quantitative PCR-based method for the titration of AAV-2 vector stocks. Mol Ther 6:272-278, 2002
5. Wang Y, Kuro O M, Sun Z: Klotho Gene Delivery Suppresses Nox2 Expression and Attenuates Oxidative Stress in Rat Aortic Smooth Muscle Cells via the cAMP-PKA Pathway. Aging Cell, June, 2012.
6. Wang Y, Sun Z: Klotho gene delivery prevents the progression of spontaneous hypertension and renal damage. Hypertension 54:810-817, 2009
7. Carter J D, Dula S B, Corbin K L, Wu R, Nunemaker C S: A Practical Guide to Rodent Islet Isolation and Assessment. Biol Proced Online, 2009
8. Gaddy D F, Riedel M J, Pejawar-Gaddy S, Kieffer T J, Robbins P D: In vivo expression of HGF/NK1 and GLP-1 From dsAAV vectors enhances pancreatic ss-cell proliferation and improves pathology in the db/db mouse model of diabetes. Diabetes 59:3108-3116
9. Wang A Y, Peng P D, Ehrhardt A, Storm T A, Kay M A: Comparison of adenoviral and adeno-associated viral vectors for pancreatic gene delivery in vivo. Hum Gene Ther 15:405-413, 2004
10. Wang Y, Sun Z: Current understanding of klotho. Ageing Res Rev 8:43-51, 2009
11. Butler A E, Janson J, Bonner-Weir S, Ritzel R, Rizza R A, Butler P C: Beta-cell deficit and increased beta-cell apoptosis in humans with type 2 diabetes. Diabetes 52:102-110, 2003
12. Leonardi O, Mints G, Hussain M A: Beta-cell apoptosis in the pathogenesis of human type 2 diabetes mellitus. Eur J Endocrinol 149:99-102, 2003
13. Medici D, Razzaque M S, Deluca S, Rector T L, Hou B, Kang K, Goetz R, Mohammadi M, Kuro O M, Olsen B R, Lanske B: FGF-23-Klotho signaling stimulates proliferation and prevents vitamin D-induced apoptosis. J Cell Biol 182:459-465, 2008
14. Shalhoub V, Ward S C, Sun B, Stevens J, Renshaw L, Hawkins N, Richards W G: Fibroblast growth factor 23 (FGF23) and alpha-klotho stimulate osteoblastic MC3T3.E1 cell proliferation and inhibit mineralization. Calcif Tissue Int 89:140-150
15. Ebato C, Uchida T, Arakawa M, Komatsu M, Ueno T, Komiya K, Azuma K, Hirose T, Tanaka K, Kominami E, Kawamori R, Fujitani Y, Watada H: Autophagy is important in islet homeostasis and compensatory increase of beta cell mass in response to high-fat diet. Cell Metab 8:325-332, 2008

What is claimed is:

1. A method of treating a diabetic condition or a diabetes-related condition in a subject in need of such treatment, the method comprising:
administering to the subject a therapeutically-effective amount of a vector comprising a nucleic acid which encodes a klotho protein or a therapeutically-effective portion of a klotho protein, wherein the klotho protein or therapeutically-effective portion of the klotho protein is expressed in vivo in pancreatic beta cells of the subject.

2. The method of claim 1, wherein the vector further comprises a promoter operatively-linked to the nucleic acid which encodes the klotho protein or the therapeutically-effective portion of a klotho protein, wherein the promoter is specific for pancreatic beta cells.

3. The method of claim 1, wherein the vector is selected from the group consisting of adeno-associated virus, nanoparticles, plasmids, and lentivirus.

4. The method of claim 1, wherein the nucleic acid which encodes the klotho protein or the therapeutically-effective portion of a klotho protein is selected from the group consisting of SEQ ID NOs:1-6, and nucleic acids which have at least 90% identity to at least one of SEQ ID NOs:1-6, and which encode the klotho protein or the therapeutically-effective portion of a klotho protein.

5. The method of claim 1, wherein the diabetic condition is Type II diabetes mellitis.

6. The method of claim 1, wherein the diabetic condition is Type I diabetes mellitis.

7. The method of claim 1, wherein the diabetes-related condition is selected from the group consisting of hyperinsulenima (pre-diabetes), obesity, peripheral arterial disease (PAD) of the arms, legs, and/or feet, foot ulcers, hypertension, diabetic neuropathy, diabetic retinopathy, diabetic kidney disease, ketoacidosis, and hyperosmolar hyperglycemic nonketotic syndrome (HHNS).

8. The method of claim 1, wherein the treatment results in at least one of (a) an increase in blood insulin levels in the subject, and (b) an increase in insulin storage levels in the subject.

9. The method of claim 1, wherein the treatment results in at least one of (a) an increase in insulin sensitivity in the subject, and (b) a decrease in insulin resistance in the subject.

10. The method of claim 1, wherein the treatment results in a decrease in average blood glucose levels in the subject.

11. The method of claim 1, wherein the treatment results in at least one of (a) a stabilization or an increase in C-peptide production in the subject, and (b) a stabilization of beta cell mass in the subject as indicated by a measurement of C-peptide production in the subject.

12. The method of claim 1, wherein the treatment results in a hemoglobin A1C value of less than about 7% in the subject.

13. A method of treating a diabetic condition or a diabetes-related condition in a subject in need of such treatment, the method comprising:
administering to the subject a therapeutically-effective amount of at least one of a klotho protein and a therapeutically-effective portion of the klotho protein, thereby mitigating the diabetic condition or diabetes-related condition in the subject.

14. The method of claim 13, wherein the klotho protein or the therapeutically-effective portion of the klotho protein is selected from the group consisting of SEQ ID NOs:7-11 and therapeutically-effective portions thereof, and therapeutically-effective proteins which have at least 90% Identity to at least one of SEQ ID NOs:7-11.

15. The method of claim 13, wherein the diabetic condition is Type II diabetes mellitis.

16. The method of claim 13, wherein the diabetic condition is Type I diabetes mellitis.

17. The method of claim 13, wherein the diabetes-related condition is selected from the group consisting of hyperinsulenima (pre-diabetes), obesity, peripheral arterial disease (PAD) of the arms, legs, and/or feet, foot ulcers, hypertension, diabetic neuropathy, diabetic retinopathy, diabetic kidney disease, ketoacidosis, and hyperosmolar hyperglycemic nonketotic syndrome (HHNS).

18. The method of claim 13, wherein the treatment results in at least one of:
(a) an increase in blood insulin levels in the subject;
(b) an increase in insulin storage levels in the subject;
(c) an increase in insulin sensitivity in the subject;
(d) a decrease in insulin resistance in the subject;
(e) a decrease in average blood glucose levels in the subject;
(f) a stabilization or an increase in C-peptide production in the subject;
(g) a stabilization of beta cell mass in the subject as indicated by a measurement of C-peptide production in the subject; and
(h) a hemoglobin A1C value of less than about 7% in the subject.


SEQUENCE LISTING (29 sequences)

SEQ ID NO: 1 - DNA, 3163 bp, Homo sapiens
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtgtctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggacacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggccgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaacggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctaga

SEQ ID NO: 2 - DNA, 3435 bp, Homo sapiens
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtgtctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggacacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtaagtcagctgacaaaaccaatcagcagtctcaccaagccctatcactagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggccgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaacggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttct

SEQ ID NO: 3 - DNA, 3460 bp, Homo sapiens
cagggaatgaatggattttcttcagcactgatgaaataaccacacgctataggaatacaatgtccaacgggggattgcaaagatctgtcatcctgtcagcacttattctgctacgagctgttactggattctctggagatggaagagctatatggtctaaaaatcctaattttactccggtaaatgaaagtcagctgtttctctatggcactttccctaaaaactttttctggggtattgggactggagcattgcaagtggaagggagttggaagaaggatggaaaaggaccttctatatgggatcatttcatccacacacaccttaaaaatgtcagcagcacgaatggttccagtgacagttatatttttctggaaaaagacttatcagccctggattttataggagtttctttttatcaattttcaatttcctggccaaggcttttccccgatggaatagtaacagttgccaacgcaaaaggtctgcagtactacagtactcttctggacgctctagtgcttagaaacattgaacctatagttactttataccactgggatttgcctttggcactacaagaaaaatatggggggtggaaaaatgataccataatagatatcttcaatgactatgccacatactgtttccagatgtttggggaccgtgtcaaatattggattacaattcacaacccatatctagtggcttggcatgggtatgggacaggtatgcatgcccctggagagaagggaaatttagcagctgtctacactgtgggacacaacttgatcaaggctcactcgaaagtttggcataactacaacacacatttccgcccacatcagaagggttggttatcgatcacgttgggatctcattggatcgagccaaaccggtcggaaaacacgatggatatattcaaatgtcaacaatccatggtttctgtgcttggatggtttgccaaccctatccatggggatggcgactatccagaggggatgagaaagaagttgttctccgttctacccattttctctgaagcagagaagcatgagatgagaggcacagctgatttctttgccttttcttttggacccaacaacttcaagcccctaaacaccatggctaaaatgggacaaaatgtttcacttaatttaagagaagcgctgaactggattaaactggaatacaacaaccctcgaatcttgattgctgagaatggctggttcacagacagtcgtgtgaaaacagaagacaccacggccatctacatgatgaagaatttcctcagccaggtgcttcaagcaataaggttagatgaaatacgagtgtttggttatactgcctggtctctcctggatggctttgaatggcaggatgcttacaccatccgccgaggattattttatgtggattttaacagtaaacagaaagagcggaaacctaagtcttcagcacactactacaaacagatcatacgagaaaatggtttttctttaaaagagtccacgccagatgtgcagggccagtttccctgtgacttctcctggggtgtcactgaatctgttcttaagcccgagtctgtggcttcgtccccacagttcagcgatcctcatctgtacgtgtggaacgccactggcaacagactgttgcaccgagtggaaggggtgaggctgaaaacacgacccgctcaatgcacagattttgtaaacatcaaaaaacaacttgagatgttggcaagaatgaaagtcacccactaccggtttgctctggattgggcctcggtccttcccactggcaacctgtccgcggtgaaccgacaggccctgaggtactacaggtgcgtggtcagtgaggggctgaagcttggcatctccgcgatggtcaccctgtattatccgacccacgcccacctaggcctccccgagcctctgttgcatgccgacgggtggctgaacccatcgacggccgaggccttccaggcctacgctgggctgtgcttccaggagctgggggacctggtgaagctctggatcaccatcaacgagcctaaccggctaagtgacatctacaaccgctctggcaacgacacctacggggcggcgcacaacctgctggtggcccacgccctggcctggcgcctctacgaccagcagttcaggccgtcacagcgcggggccgtgtcgctgtcgctgcacgcggactgggcggaacccgccaacccctatgctgactcgcactggagggcggccgagcgcttcctgcagttcgagatcgcctggttcgccgagccgctcttcaagaccggggactaccccgcggccatgagggaatacattgcctccaagcaccgacgggggctttccagctcggccctgccgcgcctcaccgaggccgaaaggaggctgctcaagggcacggtcgacttctgcgcgctcaaccacttcaccactaggttcgtgatgcacgagcagctggccggcagccgctacgactcggacagggacatccagtttctgcaggacatcacccgcctgagctcccccacgcgcctggctgtgattccctggggggtgcgcaagctgctgcggtgggtccggaggaactacggcgacatggacatttacatcaccgccagtggcatcgacgaccaggctctggaggatgaccggctccggaagtactacctagggaagtaccttcaggaggtgctgaaagcatacctgattgataaagtcagaatcaaaggctattatgcattcaaactggctgaagagaaatctaaacccagatttggattcttcacatctgattttaaagctaaatcctcaatacaattttacaacaaagtgatcagcagcaggggcttcccttttgagaacagtagttctagatgcagtcagacccaagaaaatacagagtgcactgtctgcttattccttgtgcagaagaaaccactgatattcctgggttgttgcttcttctccaccctggttctactcttatcaattgccatttttcaaaggcagaagagaagaaagttttggaaagcaaaaaacttacaacacataccattaaagaaaggcaagagagttgttagctaaactgatctgtctgcatgatagacagtttaaaaattcatcccagttccatatgctggtaacttacaggagatatacctgtattatagaaagacaatctgagatacagctgtaaccaaggtgatgacaattgtctctgctgtgtggttcaaagaacattcccttaggtgttgacatcagtgaactcagttcttggatgtaaacataaaggcttcatcctgacagtaagctatgaggattacatgctacattgcttcttaaagtttcatcaactgtattccatcattctgctttagctttcatctctaccaatagctacttgtggtacaataaattatttttaagaagaaaaaaaa

SEQ ID NO: 4 - DNA, 5032 bp, Mus musculus
cctcccggctcccgcagcatgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcaagtggacactactctctctcagtttactgacccgaatgtctatctgtgggatgtgcatcacagtaagaggcttattaaagtagacggggttgtagccaagaagagaaaaccttactgtgttgatttctctgccatccggcctcagataaccttacttcgagaaatgcgggtcacccactttcgcttctccctggactgggccctgatcttgcctctgggtaaccagacccaagtgaaccacacggttctgcacttctaccgctgcatgatcagcgagctggtgcacgccaacatcactccagtggtggccctgtggcagccagcagccccgcaccaaggcctgccacatgcccttgcaaaacatggggcctgggagaacccgcacactgctctggcgtttgcagactacgcaaacctgtgttttaaagagttgggtcactgggtcaatctctggatcaccatgaacgagccaaacacacggaacatgacctatcgtgccgggcaccacctcctgagagcccatgccttggcttggcatctgtacgatgacaagtttagggcggctcagaaaggcaaaatatccatcgccttgcaggctgactggatagaaccggcctgccctttctctcaaaatgacaaagaagtggccgagagagttttggaatttgatataggctggctggcagagcctatttttggttccggagattatccacgtgtgatgagggactggctgaaccaaaaaaacaattttcttttgccctatttcaccgaagatgaaaaaaagctagtccggggttcctttgacttcctggcggtgagtcattacaccaccattctggtagactgggaaaaggaggatccgatgaaatacaacgattacttggaggtacaggagatgactgacatcacatggctcaactctcccagtcaggtggcagtggtgccttgggggctgcgcaaagtgctcaactggctaaggttcaagtacggagacctcccgatgtatgtgacagccaatggaatcgatgatgacccccacgccgagcaagactcactgaggatctattatattaagaattatgtgaatgaggctctgaaagcctacgtgttggacgacatcaacctttgtggctactttgcgtattcacttagtgatcgctcagctcccaagtctggcttttatcgatatgctgcgaatcagtttgagcccaaaccatctatgaaacattacaggagaattattgacagcaatggcttcctgggttctggaacactgggaaggttttgtccagaagaatacactgtgtgcaccgaatgtggattttttcaaacccggaagtctttgctggtcttcatctcgtttcttgtttttacttttattatttctcttgctctcatttttcactactccaagaaaggccagagaagttataagtaatgtgaacgtctgcctggccattcgctttgggatcaagatgtacacgccgtcagccgtttgcacctctctgtgttgtgagccgcattccacacatttcgattctagaaaaccctttttgtcatgggtggtagaggttttaaacaggaattggtgagaataaaatattgcagggtgaatggtatctgaatctgctctctttggtggcaattacggaattatactcaccacagtttctacagtgccccggaatggaaggcatagaatacggtagggataacagtgccaagcagacagaagtttaaagaacaactttagggacttgtttatccatggccatttttaaattcactcctgttggggagtaacactctctcaattaccatcttaacacctggactttacctgatccagttttacaaggtgaagtagaaaaatatccagtaaaggtggccaagagccctgagtccagagcagcccattaagaagcactattcctaccaaatgctgctaatgtcaatttacaaatatacttagaaagcacattatggacatttgtattcttgtgaatgtttttgaggtgtgccctaaaccccagatccttgagggctttctcttaccaactttcctttcagagcctgcttgttggagattcttccccagcccccttcccctttccctcttgctctgccccacctcgctccacccagcttgctccagcccaaagattctttatttgtttctcattaccgaaggttgtgagccaccatgtggtttctgggatttgaactcatgacctccggaggagctgtcatgctcttaaccagcccatgttgaagattcttttgataaatattcacaaaaaataaagatgagccatgagctgttggcctcttcggaagcggaaactgagtgatttgattgaacatccttttatctttgaccagaccttggaatgaatgcaatgacctttcccacaggaagaaggaggagctctcagtcaaactgtaaagaatgcctcttcagaatatgctgtcagtgcttggatgccatgatgttcaactttcttagtcgatccggcagcaatcacagtgtgagcacactgggaacctgtccttgcggccgccgagatctaccgtgtgcttctgtgaagaggctttgacgtagcccctctttgagctcttacaccatgctactgacttctagaaaggctaattaggtcttcttctacacctaataccctaagtcttactgactctcacgggagaagtctctgtgctacacctgagtggtcttattgataaccctgataccagatcaggcaagataaatccgtcatagcaggcatggctacccttgctgccacagggtcacagcacatagctcatcaccctgttattcttcatcttgcaatgtggtatggttttcctggtgaatgatcagcttttgctgtggtattctttatacatctggacttattattgaaatcaaatgctatagaatcaatagtttattttatgtctatttttcttgatcgcagagtaatatatattaattgtaaaaaatttaagaaacaaaaactatatgtaaagaaaaaattataatataatacagagatgctgctgacagttcctatgtgttgtgttttgtatactgagatcatgtgatacgtaggcatacatcttcttgggtttttttgtttttgttttttgttttgttttgttttgttttggttttttgagatagggtttctctgtatagccctggctgtcctggaactcactttgcagaccaggctagcctcaaactcttattcatttttactgaagtaatttttctgtcattagtcttcaagagcaaaactttaatagttatggagaatattgccagaacagctcaaaactgttttatttgttggtccaatttcccattaattagttcaataataaatatcatttagaaataaaaaaaa

SEQ ID NO: 5 - DNA, 1650 bp, Mus musculus
atgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctgcttcggaccaaccttgagctttcagctattggaccctaacatgaagttccgccaattggagtctcccaacctgaggcagcttctstcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgtacaagtaagtcctttgacaaaacccagtgtcggcctcttgcttcctcac

SEQ ID NO: 6 - DNA, 3045 bp, Rattus norvegicus
atgccagcccgcgcccctcctcgccgcctgccgcggctcttgctgctccgtttgctgtcgctgcatctgctgttgctcaccctgcgcgcccgctgcctaagcgctgagccgggtcagggcgcgcagacctgggctcgctttgcgcgccctcctgtcccagaggcctctggcctcctccatgacaccttccccgatggtttcctctgggcggtaggcagcgccgcctaccagacggagggcggctggcgacagcacggcaaaggcgcgtccatctgggacactttcacccatcaccctcgggccatcccggaggactccccgatcgtcatggcgccgtcgggtgccccgttgcctcccctgccctccactggagatgtggccagcgacagttacaacaacgtctaccgcgacacagaggggctacgcgaactgggggtcacccactaccgcttctccatatcgtgggcgcgggtgctccccaatggcaccgcgggcacccccaaccgcgaggggctgcgctactaccggcggctgctggagcggctgagggagctgggcgtgcagccggtggtcactctgtaccattgggacctgccgcagcgcctgcaggacacctatggcggctgggccaatcgcgccctggccgaccatttcagggattacgccgagctctgcttccgccacttcggtggtcaggtcaagtactggatcaccattgacaacccctacgtggtggcctggcacgggtacgccaccggccgcctggccccgggcgtgcggggcagctccaggctcgggtacctggttgcccacaacctacttttggctcacgccaaagtctggcgtctctacaacacctccttccgccccactcaggggggccgggtgtccatcgccttaggttcccattggatcacccctcgaaggatgaccgactatcacatcagagagtgtcagaagtctcttgacttcgtgctaggctggtttgccaagcccatatttatcgatggcgactacccaaagagtatgaagaacaacctctcatccctgctgcccgattttactgaatccgagaagagattcatcaggggaacagctgacttctttgctctctccttcggaccaaccttgagctttcaactactggaccccagcatgaagttccgccagctggagtctcccagcctgaggcagcttctgtcttggatagatctggagtataaccaccctcagatatttattgtagaaaatggctggtttgtctcggggaccaccagaagagatgatgccaaatacatgtattatctcaagaagttcataatggaaagcttaaaagccatcaggctggatggggtcgacgtcattgggtacaccgcatggtccctcatggatggtttcgagtggcacaggggctacagcatccgacgaggactcttctatgtcgactttctgagtcaggacaaggagttgttgcccaagtcttcggccttgttctaccaaaagctgatagagaacaatggcttccctcctttacctgagaaccagccccttgaagggacgtttccctgtgactttgcttggggagtggttgacaactacattcaagtggaccccactctctctcagtttactgacccgaatgtctatctgtgggatgtgcatcacagtaagaggcttattaaagtagacggggttgtagccaagaagagaaaaccttactgtgttgatttctctgccatccggcctcagataaccttacttcgagaaatgcgggtcacccactttcggttctccctagactgggccctgatcttgcctctgggtaaccagactcaagtgaaccgcacggttctgcacttctaccgctgtatggtcagcgagctggtgcacgccaacatcactccagtggtggccctgtggcaaccagcaaccccacaccaaggcctgccacatgcccttgcaaaacatggggcctgggagaacccgcacactgccctggcatttgcagactacgcaaacctgtgctttgaagagctaggccactgggtcaagttctggatcaccataaatgagccaaactcacggaacatgacataccgtgcggggcaccacctgcttaaggctcatgccttggcatggcacctgtacgatgacaaatttagggcggctcagaaaggcaaaatatccatcgccttgcaggttgactggatagaaccggcctgcccattctctcagaaggacaaagaagtggcggagagagttttggaatttgatgtaggctggctggcagagcccattttcggctccggagattatccgcatgtgatgagggaatggctgaaccaaaaaaacaactttcttctgccctatttcacggaagatgagaaaaagctaatccggggttcctttgacttcctggcgttgagccattacaccaccatccttgtagactgggaaaaggaggatccgataaaatacaacgattacctggaggtgcaggagatgactgacatcacgtggctcaactcccccaatcaggtggcggtggtgccttgggggctgcgcaaagcgctcaactggctaaggttcaagtatggagacctcccgatgttcgtgacagccaatggcattgatgacgatccccatgccgagcaagactcactgaggatgtattatattaagaattacgtgaatgaggctctgaaagcctacgtgttggacggcatcaacctttgtggctactttgcgtattcgcttagtgaccgctcagttcccaagtccggcttttatcgatacgctgcgaatcagttcgagcccaaaccgtctattaaacattacaggaaaattattgacaacaacggcttcctgggttctggaacgctgggaaggttttgtccggaagagtacaccgtgtgcaccggatgcggcttttttcaaacccggaagtccctgctggccttcatctcgtttctagtttttgctttcgttacttctctcgctcttatttattactactccaagaaaggccggagacgttataaataa

SEQ ID NO: 7 - protein, 549 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVSQLTKPISSLTKPYH

SEQ ID NO: 8 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 9 - protein, 550 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVSPLTKPSVGLLLPH

SEQ ID NO: 10 - protein, 1014 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVDTTLSQFTDPNVYLWDVHHSKRLIKVDGVVAKKRKPYCVDFSAIRPQITLLREMRVTHFRFSLDWALILPLGNQTQVNHTVLHFYRCMISELVHANITPVVALWQPAAPHQGLPHALAKHGAWENPHTALAFADYANLCFKELGHWVNLWITMNEPNTRNMTYRAGHHLLRAHALAWHLYDDKFRAAQKGKISIALQADWIEPACPFSQNDKEVAERVLEFDIGWLAEPIFGSGDYPRVMRDWLNQKNNFLLPYFTEDEKKLVRGSFDFLAVSHYTTILVDWEKEDPMKYNDYLEVQEMTDITWLNSPSQVAVVPWGLRKVLNWLRFKYGDLPMYVTANGIDDDPHAEQDSLRIYYIKNYVNEALKAYVLDDINLCGYFAYSLSDRSAPKSGFYRYAANQFEPKPSMKHYRKIIDSNGFLGSGTLGRFCPEEYTVCTECGFFQTRKSLLVFISFLVFTFIISLALIFHYSKKGQRSYK

SEQ ID NO: 11 - protein, 1014 aa, Rattus norvegicus
MPARAPPRRLPRLLLLRLLSLHLLLLTLRARCLSAEPGQGAQTWARFARPPVPEASGLLHDTFPDGFLWAVGSAAYQTEGGWRQHGKGASIWDTFTHHPRAIPEDSPIVMAPSGAPLPPLPSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWRLYNTSFRPTQGGRVSIALGSHWITPRRMTDYHIRECQKSLDFVLGWFAKPIFIDGDYPKSMKNNLSSLLPDFTESEKRFIRGTADFFALSFGPTLSFQLLDPSMKFRQLESPSLRQLLSWIDLEYNHPQIFIVENGWFVSGTTRRDDAKYMYYLKKFIMESLKAIRLDGVDVIGYTAWSLMDGFEWHRGYSIRRGLFYVDFLSQDKELLPKSSALFYQKLIENNGFPPLPENQPLEGTFPCDFAWGVVDNYIQVDPTLSQFTDPNVYLWDVHHSKRLIKVDGVVAKKRKPYCVDFSAIRPQITLLREMRVTHFRFSLDWALILPLGNQTQVNRTVLHFYRCMVSELVHANITPVVALWQPATPHQGLPHALAKHGAWENPHTALAFADYANLCFEELGHWVKFWITINEPNSRNMTYRAGHHLLKAHALAWHLYDDKFRAAQKGKISIALQVDWIEPACPFSQKDKEVAERVLEFDVGWLAEPIFGSGDYPHVMREWLNQKNNFLLPYFTEDEKKLIRGSFDFLALSHYTTILVDWEKEDPIKYNDYLEVQEMTDITWLNSPNQVAVVPWGLRKALNWLRFKYGDLPMFVTANGIDDDPHAEQDSLRMYYIKNYVNEALKAYVLDGINLCGYFAYSLSDRSVPKSGFYRYAANQFEPKPSIKHYRKIIDNNGFLGSGTLGRFCPEEYTVCTGCGFFQTRKSLLAFISFLVFAFVTSLALIYYYSKKGRRRYK

SEQ ID NO: 12 - DNA, 20 bp, Mus musculus
aaatgctcagccaaggacaa

SEQ ID NO: 13 - DNA, 22 bp, Mus musculus
ggactttgctgtttgacccatt

SEQ ID NO: 14 - DNA, 20 bp, Mus musculus
cctgttggtgcacttcctac

SEQ ID NO: 15 - DNA, 20 bp, Mus musculus
cctgttggtgcacttcctac

SEQ ID NO: 16 - DNA, 22 bp, Mus musculus
agccctaagtgatccgctacaa

SEQ ID NO: 17 - DNA, 20 bp, Mus musculus
ccaccccagtttacaagctc

SEQ ID NO: 18 - DNA, 20 bp, Mus musculus
acgggtcctcttgttttcct

SEQ ID NO: 19 - DNA, 20 bp, Mus musculus
aagcccgtggaagccattag

SEQ ID NO: 20 - DNA, 24 bp, Mus musculus
ggtcattttcattgtgctcctgag

SEQ ID NO: 21 - DNA, 20 bp, Mus musculus
taaagaagaggaggcggtaa

SEQ ID NO: 22 - DNA, 20 bp, Mus musculus
taagtgtcccatgtcagcaa

SEQ ID NO: 23 - DNA, 19 bp, Mus musculus
cgagcgctacaagggtgag

SEQ ID NO: 24 - DNA, 19 bp, Mus musculus
ccggatgatcttgaccaac

SEQ ID NO: 25 - DNA, 22 bp, Mus musculus
aggtcatcactattggcaacga

SEQ ID NO: 26 - DNA, 27 bp, Mus musculus
cacttcatgatggaattgaatgtagtt

SEQ ID NO: 27 - DNA, 20 bp, Mus musculus
aaatgctcagccaaggacaa

SEQ ID NO: 28 - DNA, 22 bp, Mus musculus
ggactttgctgtttgacccatt

SEQ ID NO: 29 - DNA, 25 bp, Mus musculus
catgttgaaacaataacctggaaga